(19) United States
(12) Patent Application Publication
(10) Pub. No.: US 2005/0288270 A1
(43) Pub. Date: Dec. 29, 2005

(54) New aminopyridine derivatives and their use as pharmaceuticals

(75) Inventors: Charlotte Moira Norfor Allerton, Sandwich (GB); Andrew Simon Cook, Sandwich (GB); David Hepworth, Sandwich (GB); Duncan Charles Miller, Sandwich (GB)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 11/138,708

(22) Filed: May 26, 2005

Related U.S. Application Data
(60) Provisional application No. 60/585,133, filed on Jul. 1, 2004.

(30) Foreign Application Priority Data
May 27, 2004 (GB) 0411891.5
Jun. 3, 2004 (GB) 0412463.2

(51) Int. Cl. A61K 31/541; A61K 31/5377; A61K 31/4545; A61K 31/4439; C07D 43/02; C07D 413/02; C07D 417/02

(52) U.S. Cl. 514/210.2; 514/227.5; 514/235.2; 514/318; 544/60; 544/124; 546/193; 546/268.4

(57) ABSTRACT

The present invention provides for compounds of formula (I): 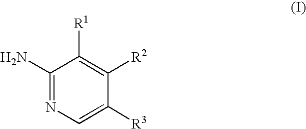 which are a class of dopamine agonists, more particularly a class of agonists that are selective for D3 over D2. These compounds are useful for the treatment and/or prevention of sexual dysfunction, for example female sexual dysfunction (FSD), in particular female sexual arousal disorder (FSAD), hypoactive sexual desire disorder (HSDD; lack of interest in sex), female orgasmic disorder (FOD; inability to achieve orgasm); and male sexual dysfunction, in particular male erectile dysfunction (MED). Male sexual dysfunction as referred to herein is meant to include ejaculatory disorders such as premature ejaculation, anorgasmia (inability to achieve orgasm) or desire disorders such as hypoactive sexual desire disorder (HSDD; lack of interest in sex). These compounds are also useful in treating neuropsychiatric disorders and neurodegenerative disorders.

The present invention relates to a class of dopamine agonists, more particularly a class of agonists that are selective for D3 over D2. These compounds are useful for the treatment and/or prevention of sexual dysfunction, for example female sexual dysfunction (FSD), in particular female sexual arousal disorder (FSAD), hypoactive sexual desire disorder (HSDD; lack of interest in sex), female orgasmic disorder (FOD; inability to achieve orgasm); and male sexual dysfunction, in particular male erectile dysfunction (MED). Male sexual dysfunction as referred to herein is meant to include ejaculatory disorders such as premature ejaculation, anorgasmia (inability to achieve orgasm) or desire disorders such as hypoactive sexual desire disorder (HSDD; lack of interest in sex). These compounds are also useful in treating neuropsychiatric disorders and neurodegenerative disorders.

The present invention provides for compounds of formula (I): 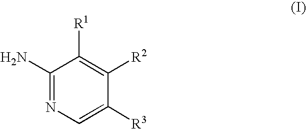
wherein:
R¹ is selected from H and (C₁-C₆)alkyl;
R² is selected from H and (C₁-C₆)alkyl;
R³ is selected from: 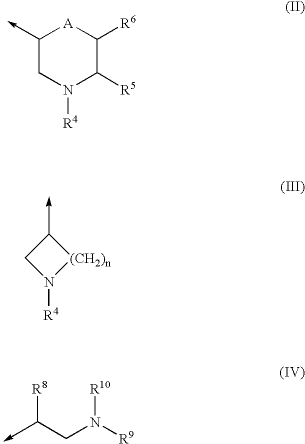
   wherein:
   A represents O, S or CH₂;
   n is 1 or 2;
   R⁴ is selected from H and (C₁-C₆)alkyl; wherein said (C₁-C₆)alkyl may be optionally substituted with 1 or 2 substituents each independently selected from (C₁-C₆)alkyl, OR⁷, phenyl, substituted phenyl and heteroaryl;
   R⁵ is selected from H and (C₁-C₆)alkyl; wherein said (C₁-C₆)alkyl may be optionally substituted with 1 or 2 OR⁷ groups;
   R⁶ is selected from H and (C₁-C₆)alkyl;
   R⁷ is selected from H and (C₁-C₆)alkyl; wherein said (C₁-C₆)alkyl may be optionally substituted with a phenyl, or a substituted phenyl group;
   R⁸ is selected from H, methyl, ethyl, methoxy, and ethoxy;
   R⁹ represents (C₁-C₆)alkyl; and
   R¹⁰ is selected from H and (C₁-C₆)alkyl; wherein said (C₁-C₆)alkyl may be optionally substituted with 1 or 2 substituents each independently selected from OR⁷, phenyl or substituted phenyl;
      wherein heteroaryl means a 5 to 7 membered aromatic ring, containing from 1 to 4 heteroatoms, said heteroatoms each independently selected from O, S and N; said heteroaryl may be optionally substituted with 1 or more substituents each independently selected from (C₁-C₆)alkyl, halo and OR⁷, each substituent may be the same or different; and
      wherein substituted phenyl means phenyl substituted with 1 or more substituents each independently selected from (C₁-C₆)alkyl, halo and OR⁷, each substituent may be the same or different;
      with the proviso that:
when R¹ and R² are H, R³ is moiety (II), A is O, R⁵ is H or (C₁-C₆)alkyl, and R⁶ is H or (C₁-C₆)alkyl, then R⁴ cannot be n-propyl;
   and pharmaceutically acceptable salts, solvate, polymorphs and prodrugs thereof.

Unless otherwise indicated, (C₁-C₆)alkyl may be straight chain or branched.

Suitable heteroaryl groups include pyridinyl, pyrimidinyl, pyridazinyl and pyrazinyl.

Unless otherwise indicated, the term halo means fluoro, chloro, bromo or iodo.

Unless otherwise indicated, the term substituted means substituted by one or more defined groups. In the case where groups may be selected from a number of alternatives groups, the selected groups may be the same or different.

The pharmaceutically acceptable salts of the compounds of the formula (I) include the acid addition and the base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Hemisalts of acids may also be formed, for example, hemisulphate.

For a review on suitable salts, see Handbook of Pharmaceutical Salts: Properties, Selection, and Use by Stahl and Wermuth (Wiley-VCH, 2002).

Pharmaceutically acceptable salts of compounds of formula I may be prepared by one or more of three methods:
(i) by reacting the compound of formula I with the desired acid or base;
(ii) by removing an acid- or base-labile protecting group from a suitable precursor of the compound of formula I or by ring-opening a suitable cyclic precursor, for example, a lactone or lactam, using the desired acid or base; or
(iii) by converting one salt of the compound of formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of the invention may also exist in unsolvated and solvated forms. The term ‘solvate’ is used herein to describe a molecular complex comprising the compound of the invention and one or more pharmaceutically acceptable solvent molecules, for example, ethanol. The term ‘hydrate’ is employed when said solvent is water.

The pharmaceutically acceptable solvates of the compounds of the formula (I) include the hydrates thereof.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Hereinafter all references to compounds of formula I include references to the salts and solvates thereof.

The compounds of the invention include compounds of formula I as hereinbefore defined, including all polymorphs and crystal habits thereof, prodrugs and isomers thereof (including optical, geometric and tautomeric isomers) as hereinafter defined and isotopically-labeled compounds of formula I.

Included within the scope of the present invention are all stereoisomers, geometric isomers and tautomeric forms of the compounds of formula I, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

A compound of the formula (I) contains one or more asymmetric carbon atoms and therefore exists in two or more stereoisomeric forms. Furthermore, the skilled person will understand that moiety (II) encompasses all stereoisomeric and distereoisomeric forms, in particular:

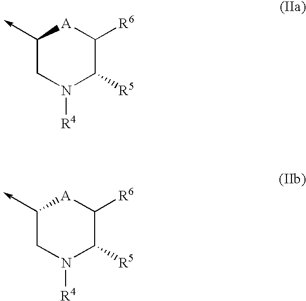

Separation of diastereoisomers may be achieved by conventional techniques, e.g. by fractional crystallisation, chromatography or H.P.L.C. of a stereoisomeric mixture of a compound of the formula (I) or a suitable salt or derivative thereof. An individual enantiomer of a compound of the formula (I) may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention includes all pharmaceutically acceptable isotopically-labelled compounds of formula I wherein one or more atoms are replaced by atoms having the same atomic number, but an atomic mass or mass number different from the atomic mass or mass number which predominates in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of formula I, for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Isotopically-labeled compounds of formula I can generally be prepared by conventional techniques known to those skilled in the art or by processes analogous to those described in the accompanying Examples and Preparations using an appropriate isotopically-labeled reagent in place of the non-labeled reagent previously employed.

Pharmaceutically acceptable solvates in accordance with the invention include those wherein the solvent of crystallization may be isotopically substituted, e.g. D₂O, d₆-acetone, d₆-DMSO.

The following embodiments of the invention are particularly favoured:
Preferably R¹ is selected from H, methyl and ethyl
More preferably R¹ is selected from H and methyl
Most preferably R¹ is H.
Preferably R² is selected from H, methyl and ethyl
More preferably R² is selected from H and methyl
Most preferably R² is H.

When R³ is moiety (II):
Moieties (IIa) and (IIb) are preferred.
Preferably A is O or CH₂
More preferably A is 0
Preferably R⁴ is (C₁-C₆)alkyl optionally substituted with a phenyl or a substituted phenyl group.
More preferably R⁴ is (C₁-C₄)alkyl optionally substituted with phenyl
Even more preferably R⁴ is selected from methyl, ethyl, n-propyl or n-butyl
Most preferably R⁴ is selected from methyl, ethyl, and n-propyl
In a first preferred embodiment, R⁵ is selected from H and (C₁-C₄)alkyl; wherein said (C₁-C₄)alkyl may be optionally substituted with 1 or 2 OR⁷ groups
More preferably R⁵ is selected from H, methyl and ethyl; wherein said methyl and said ethyl may be optionally substituted with an OR⁷ group
Most preferably R⁵ is selected from H, methyl and ethyl.
In a second preferred embodiment, R⁵ is (C₁-C₄)alkyl optionally substituted with 1 or 2 OR⁷ groups
More preferably R⁵ is selected from methyl and ethyl; wherein said methyl and said ethyl may be optionally substituted with an OR⁷ group.
Most preferably R⁵ is selected from methyl and ethyl.
Preferably R⁶ is selected from H, methyl and ethyl
More preferably R⁶ is selected from H and methyl
Most preferably R⁶ is H
Preferably R⁷ is selected from H and (C₁-C₄)alkyl; wherein said (C₁-C₄)alkyl is optionally substituted with 1 or 2 phenyl or substituted phenyl groups
More preferably R⁷ is selected from H, methyl and ethyl; wherein said methyl and said ethyl are optionally substituted with a phenyl group
Most preferably R⁷ is selected from H and (CH₂)phenyl

When R³ is moiety (III):
Preferably n is 1
Preferably R⁴ represents (C₁-C₄)alkyl, optionally substituted by 1 or 2 phenyl, substituted phenyl or heteroaryl groups.
More preferably R⁴ represents ethyl, propyl or butyl, said groups being optionally substituted by a phenyl group.
Most preferably R⁴ represents ethyl or propyl, said groups being optionally substituted by a phenyl group.

When R³ is moiety (IV):
Preferably R⁸ is selected from H, methyl and methoxy.
More preferably R⁸ is selected from H and methoxy.
Most preferably R⁸ is H.
Preferably R⁹ is selected from (C₁-C₄)alkyl.
More preferably R⁹ is selected from methyl, ethyl and n-propyl.
Most preferably R⁹ is n-propyl.
Preferably R¹⁰ is selected from H and (C₁-C₃)alkyl; wherein said (C₁-C₃)alkyl may be optionally substituted with 1 or 2 OR⁷ or phenyl groups.
More preferably R¹⁰ is selected from H and methyl.
Most preferably R¹⁰ is H.
Preferably R³ is selected from moieties (II) and (III)
More preferably R³ is selected from moieties (IIa), (IIb), and (III)
Most preferably R³ is selected from moieties (IIa) and (IIb).
Preferably heteroaryl is a 5 or 6 membered aromatic ring, containing from 1 to 3 heteroatoms, said heteroatom independently selected from O and N;
More preferably heteroaryl is a 5 or 6 membered aromatic ring, containing from 1 to 2 nitrogen atoms.

Particularly preferred are compounds (and salts thereof) of the present invention are exemplified herein; more preferred are:
5-(Morpholin-2-yl)pyridin-2-amine (Example 2);
5-[4-(3-Phenylpropyl)morpholin-2-yl]pyridin-2-amine (Example 3);
5-[(2R, 5S)-5-Methylmorpholin-2-yl]pyridine-2-amine (Example 7a);
5-[(2S,5S)-5-Methyl-4-(3-phenylpropyl)morpholin-2-yl]pyridin-2-amine (Example 9);
5-[(2S,5S)-4-Butyl-5-methylmorpholin-2-yl]pyridin-2-amine (Example 10);
5-{(2R,5S)-5-[(Benzyloxy)methyl]-4-propylmorpholin-2-yl}pyridin-2-amine (Example 13);
[6-(6-Aminopyridin-3-yl)-4-propylmorpholin-3-yl]methanol (Example 14);
4-Methyl-5-(4-Propylmorpholin-2-yl)pyridin-2-amine (Examples 18 & 19);
5-[(2S,5S)-4,5-Diethylmorpholin-2-yl]pyridin-2-amine (Example 21);
5-[(2R,5S)-4,5-Diethylmorpholin-2-yl]pyridin-2-amine (Example 22);
5-(2R,5S)-(4-ethyl-5-methylmorpholin-2-yl)-pyridin-2-ylamine (Example 25).

In an alternative embodiment, the invention additionally comprises the compounds (+)-5-(4-propylmorpholin-2-yl)-1,3-thiazol-2-amine and (−)-5-(4-propylmorpholin-2-yl)-1,3-thiazol-2-amine (Examples 26 and 27).

Compounds of the invention may be prepared, in known manner, in a variety of ways.

The routes below illustrate methods of synthesising compounds of formula (I); the skilled man will appreciate that other methods may be equally as practicable.

Throughout the schemes the protected nitrogen of the 2-aminopyridine group is signified as PGN, and Hal represents a halogen selected from Cl, Br, or I.

Compounds of formula (I) wherein R¹, R², R⁴ and R⁶ are as defined above, and R³, R⁵ and A are as described herein, may be prepared according to reaction scheme 1. 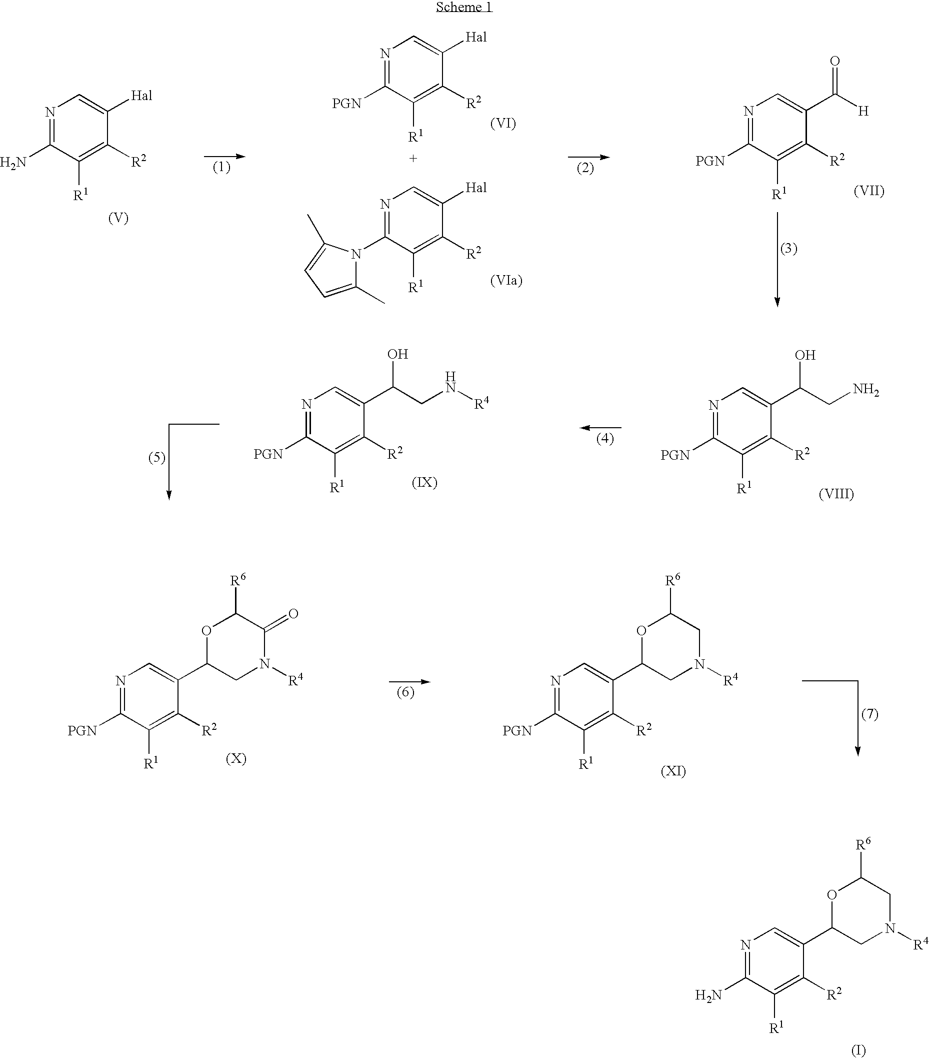
Reaction Step 1. Aminopyridine Protection

Compounds of formula (V), wherein, for example, NPG is the 2,5-dimethylpyrrole system [as described in J. Chem. Soc. Perkin Trans. 1, 1984, 2801-2807, and as illustrated by the compound of formula (VIa)], may be introduced through reaction of an aminopyridine of formula (V) with 1-2 equivalents of 2,5-hexanedione in toluene at reflux with azeotropic removal of water and an acid catalyst, such as para-toluenesulfonic acid.

Reaction Step 2. Halide to Aldehyde

Aromatic halide of formula (VI) may be converted into aldehydes of formula (VII) by, for example, generation of an organometallic reagent from a halogenated pyridine of formula (VI), followed by reaction with a formylating agent such as dimethylformamide or morpholine-4-carbaldehyde.

Suitable organometallic pyridine derivatives include Grignard (organomagnesium) or organolithium reagents, which may be prepared from the bromide (or iodide) by halogen-metal exchange. Typical conditions comprise addition of isopropylmagnesium chloride (or butyllithium) to the bromide (VI) in an anhydrous ethereal solvent such as tetrahydrofuran at room temperature (may require heating in certain cases when isopropylmagnesium chloride is used as the metallating agent) or below (e.g. −78° C. when butyllithium is used) to perform the halogen metal exchange reaction, followed by addition of the formylating agent at 0° C. or lower.

Reaction Step 3. Conversion of Aldehyde to Aminoalcohol

Compounds of formula (VIII) may be prepared by reaction of an aldehyde of formula (VII) with a cyanide source, such as potassium cyanide or trimethylsilylcyanide, or with nitromethane and a base, such as sodium hydroxide, to form an intermediate adduct which may be reduced by treatment with borane, lithium aluminium hydride or hydrogenation in an ethereal solvent. Typical conditions comprise reacting 1.0 equivalents of aldehyde in 1.5 equivalents of 3M HCl with sodium sulfite (1.5 equivalents) followed by potassium cyanide (1.5 equivalents) at room temperature. The resulting cyanohydrin intermediate is then reduced by treatment with 1.2-3.0 equivalents of borane in THF at reflux, followed by treatment with a strong acid to hydrolyse the initially formed boron complex of the product. The skilled person will be aware that other non-acidic methods are available for breaking the boron complex e.g. treatment with diethanolamine.

Reaction Step 4. Reductive Amination

Compounds of formula (IX) may be prepared from compounds of formula (VIII) by employing standard amide bond forming conditions followed by reduction of the intermediate amide with a hydride reducing agent such as borane or lithium aluminium hydride.

For example, acid chlorides in the presence of a suitable base such as triethylamine or 4-methylmorpholine may be used for the amide forming stage. Typical reaction conditions comprise 1.0 equivalents of amine (VIII), 1.2-2.0 equivalents of base (preferably triethylamine), 1.1-1.3 equivalents of acid chloride in dichloromethane at 25° C. Reducing agents such as borane or lithium aluminium hydride can be used for the amide reduction stage. Typical conditions comprise 1.0 equivalents of amide, 1.2-3.0 equivalents of borane in THF at reflux, followed by treatment with a strong acid to hydrolyse the initially formed boron complex of the product. The skilled person will be aware that other non-acidic methods are available for breaking the boron complex e.g. treatment with diethanolamine.

Compounds of formula (IX) can also be made by reductive amination of compounds of formula (VIII) with a suitable aldehyde (1 equivalent or more) in the presence of a hydride reducing agent such as sodium cyanoborohydride or sodium triacetoxyborohydride (1 equivalent or more) in an alcoholic solvent such as ethanol at room temperature.

Reaction Step 5. Morpholinone Formation

Compounds of formula (X) may be prepared by reaction of compounds of formula (IX) with chloroacetyl chloride or 2-substituted □-chloroacyl chlorides (such as 2-chloropropionyl chloride or 2-chlorobutyryl chloride) in the presence of a base such as triethylamine, sodium carbonate or potassium hydroxide. Typical conditions comprise 1.0 equivalents of amine (IX), 1.0-1.3 equivalents of acid chloride, 1.2-2.0 equivalents of triethylamine in dichloromethane at 25° C., the crude reaction mixture is then dissolved in IPA with 1.2-3.0 equivalents of aqueous potassium hydroxide.

Reaction Step 6. Morpholinone Reduction

Compounds of formula (XI) may be prepared by reaction of compounds of formula (X) with reducing agents such as borane or lithium aluminium hydride. Typical conditions comprise 1.0 equivalents of amide (X), 1.2-3.0 equivalents of borane in THF at reflux, followed by treatment with a strong acid to hydrolyse the initially formed boron complex. The skilled person will be aware that other non-acidic methods are available for breaking the boron complex e.g. treatment with diethanolamine.

Reaction Step 7. Aminopyridine Deprotection

Compounds of formula (I) may be prepared from compounds of formula (XI) by deprotection. The nature of this reaction will depend upon the protecting group selected for use.

For example, when the 2,5-dimethylpyrrole system is used to protect the aminopyridine group it may be deprotected by treatment with hydroxylamine. Typical conditions comprise 1.0 equivalents of compound (XI) and 5 equivalents of hydroxylamine hydrochloride in ethanol at reflux

Alternatively, compounds of formula (IX), wherein R¹, R², and R⁴ are as defined above, may be prepared according to reaction scheme 2. 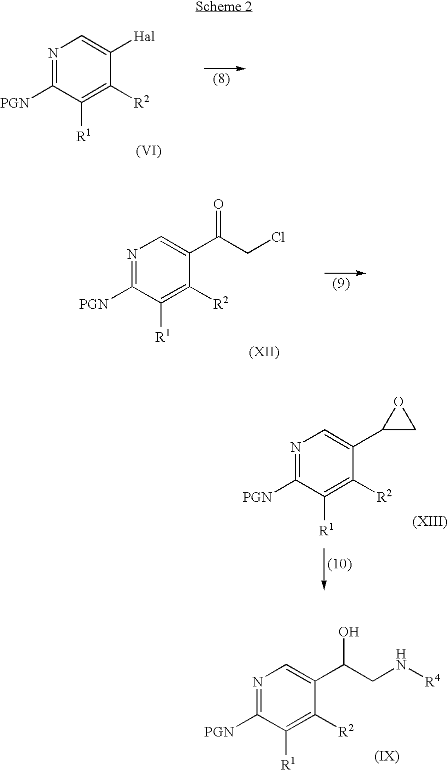
Reaction Step 8. Halide to Chloroketone

Chloroketones of formula (XII) may be formed from halides of formula (VI) via generation of a reactive organometallic intermediate. Suitable organometallic pyridine derivatives include Grignard (organomagnesium) or organolithium reagents, which may be prepared from the bromide (or iodide) by halogen-metal exchange. Thus, treatment of (VI) with 1.1 (or more) equivalents of butyllithium in an ethereal solvent such as tert-butylmethyl ether at low temperature (−78° C.) affords an organometallic intermediate which can then be treated with 2-chloro-N-methoxy-N-methylacetamide to provide chloroketones of formula (XII).

Reaction Step 9. Chloroketone to Epoxide

Chloroketones of formula (XII) may be converted in epoxides of formula (XIII) via reduction to an intermediate chlorohydrin and base promoted epoxide formation. Thus, reaction of (XII) with sodium borohydride (0.3 equivalents or more) in dioxan with subsequent treatment with excess sodium hydroxide solution affords epoxides of formula (XIII).

Enantiomerically pure, or enantiomerically enriched epoxides of formula (XIII) may be obtained by employing an asymmetric reducing agent. For example, reaction of chloroketones of general formula (XIII) with (−)-B-chlorodiisopinocampheylborane (1.5 or more equivalents) in tetrahydrofuran at low temperature (e.g −30° C.) and subsequent treatment of the intermediate chlorohydrin with sodium hydroxide affords enantiomerically enriched epoxides of formula (XIII).

Reaction Step 10. Epoxide Opening

Epoxides of formula (XIII) when heated with suitable primary amines in an inert solvent such as DMSO at 90° C. afford compounds of formula (IX).

Compounds of formula (I) wherein R¹, R², R⁴, and R⁵ are as defined above, and R³, R⁶ and A are as described herein, may be prepared according to reaction scheme 3. 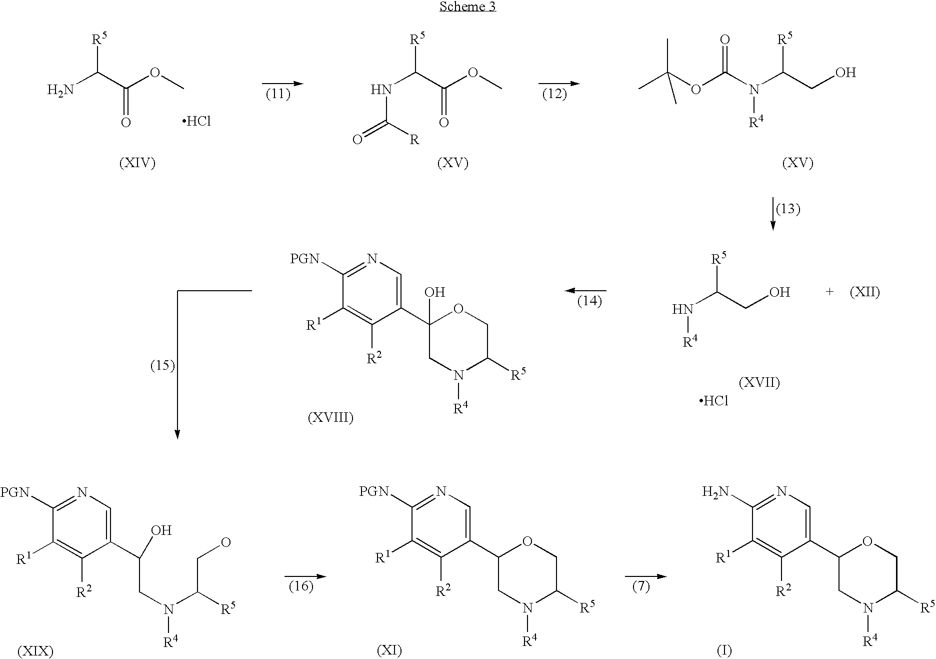
Reaction Step 11. Amide Formation

Compounds of the formula (XV) may be prepared by reacting an amino acid ester of the formula (XIV) with acid chlorides (R═(C₁-C₆)alkyl) in the presence of a suitable base such as triethylamine and 4-methylmorpholine (or other suitable amide bond forming conditions). Typical reaction conditions comprise 1 equivalent amino acid ester (XIV), 1 equivalent of acid chloride and 3 equivalents of base in dichloromethane at 25° C. Examples of compounds of formula (XV) are also commercially available.

Reaction Step 12. Amide and Ester Reduction then N-Boc Formation

Compounds of the formula (XVI) may be prepared by reacting compounds of the formula (XV) with borane-THF complex, followed by treatment with a strong acid (e.g 5M HCl) to hydrolyse the resulting boron complexes with the product. Other non-acidic methods are available for breaking the boron complex e.g. treatment with diethanolamine. This is followed by t-butyloxycarbonyl protection of the formed amine. Typical reaction conditions comprise 1 equivalent of the amide (XV) with 3 equivalents of BH₃-THF in THF at reflux, cooling, cautious addition of 6M aqueous HCl, and heating to reflux for a further 6 h. Subsequent evaporation of solvent, redissolution in a methanol:water (8:1) mix, and addition of 5 equivalents of a base such as potassium hydroxide and 1.5 equivalents of di-tert-butyl dicarbonate, and stirring of the mixture for 72 hours.

Reaction Step 13 N-Boc Deprotection

Compounds of the formula (XVII) may be prepared by reacting compounds of the formula (XVI) with an organic solution of HCl. Typical reaction conditions comprise 1 equivalent of the carbamate (XVI) and a 1-10 equivalents of a 4M solution of HCl in dioxan at 25° C. Examples of compounds of formula (XVII) are also commercially available.

Reaction Step 14. Chloroketone Addition

Compounds of the formula (XVIII) may be prepared by reacting compounds of the formula (XVII) with an □-halo ketone of formula (XII), if necessary in the presence of a base such as triethylamine or 4-methylmorpholine. Typical conditions comprise 1 equivalent of the aminoalcohol (XVII) with 1-3 equivalents of triethylamine and 1 equivalent of a compound of formula (XII) at 65° C.

Reaction Step 15. Reduction to Diol

Morpholinol intermediates of formula (XVIII) can be reduced to diols of formula (XIX) by reaction with a hydride reducing agent such as sodium borohydride (1 equivalent or more) in an alcoholic solvent such as ethanol at room temperature.

Reaction Step 16. Mor Pholine Ring Closure

Diol compounds of formula (XIX) can be ring-closed to morpholine compounds of formula (XI) using a number of methods. For example treating a dichloromethane solution of (XIX) with excess concentrated sulfuric acid at room temperature will effect cyclisation.

Alternatively, the ring closure may be effected using Mitsunobu-type conditions employing the using of 1.1 equivalents of a dialkyl azodicarboxylate reagent, such as diispropyl azodicarboxylate (DIAD), and 1.1 equivalents of triphenylphosphine in an inert solvent such as tetrahydrofuran.

A further alternative is to use a sulfonylating agent, such as p-toluenesulfonylimidazole (1 equivalent) in the presence of strong base such as sodium hydride in an inert solvent such as tetrahydrofuran, as described in Org. Lett. 2004, 6(6), 1045-1047.

Reaction Step 7. Aminopyridine Deprotection

Compounds of formula (I) may be prepared from compounds of formula (XI) by deprotection. The nature of this reaction will depend upon the protecting group selected for use.

For example, when the 2,5-dimethylpyrrole system is used to protect the aminopyridine group it may be deprotected by treatment with hydroxylamine. Typical conditions comprise 1.0 equivalents of compound (XI) and 5 equivalents of hydroxylamine hydrochloride in ethanol at reflux

Alternatively, in some cases it may prove advantageous to deprotect the aminopyridine group (PGN) prior to ring closure to form the morpholine group. This is most likely to be the case when acidic conditions are used to effect the cyclisation. In this instance, compounds of formula (I) wherein R¹, R², R⁴, R⁵ and R⁶ are as defined above, and R³ and A are as described herein, may be prepared from compounds of formula (XIX) according to reaction scheme 4. 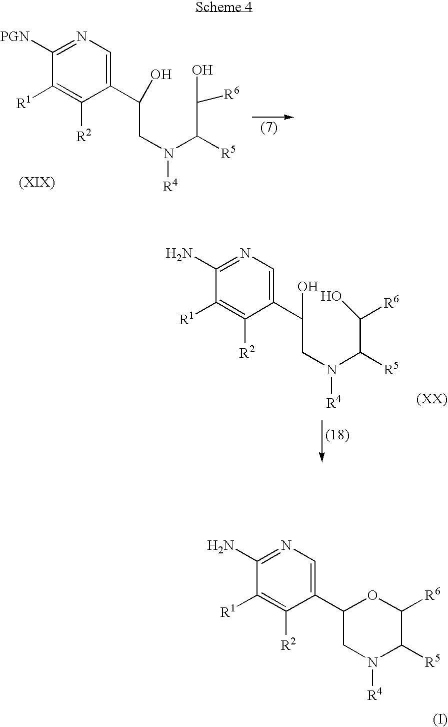
Reaction Step 7. Amino Pyridine Deprotection

Compounds of formula (XX) may be prepared from compounds of formula (XIX) by deprotection. For example, when the 2,5-dimethylpyrrole system is used to protect the aminopyridine group it may deprotected by treatment with hydroxylamine. Typical conditions comprise 1.0 equivalents of compound (XIX) and 5 equivalents of hydroxylamine hydrochloride in ethanol at reflux.

Reaction Step 18. Morpholine Ring Closure

Compounds of formula (I) may then be prepared by cyclisation of compounds of formula (XX) by treatment with acid. Typical conditions employ concentrated sulfuric acid and dichloromethane as solvent at room temperature or above.

Other methods, such as those described for Reaction Step 16 in scheme 3 may also be used to form the morpholine ring.

Scheme 5 describes an alternative method for conversion compounds of formula (XVIII) into compounds of formula (XI), wherein R¹, R², R⁴, R⁵ and R⁶ are as defined above.

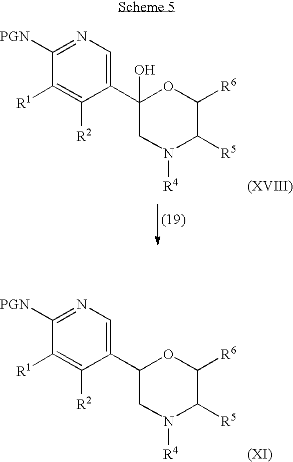

Compounds of formula (XI) may be formed from compounds of the formula (XVIII) by reaction step 19—reaction of a compound of formula (XVIII) with an hydride source such as triethylsilane and an acidic or Lewis acidic reagent such as trimethylsilyltriflate. Typical conditions comprise addition of 5-10 equivalents of triethylsilane to 1 equivalent of the morpholinol (XVIII) in dichloromethane at −78° C. followed by addition of 2 equivalents of trimethylsilyltriflate.

Similarly, if the protecting group is absent from the compound of formula (XVIII), this process step provides an alternative route to compounds of formula (I).

An alternative procedure for the formation of compounds of formula (XIX) is shown in Scheme 6, wherein R¹, R², R⁴, R⁵ and R⁶ are as defined above.

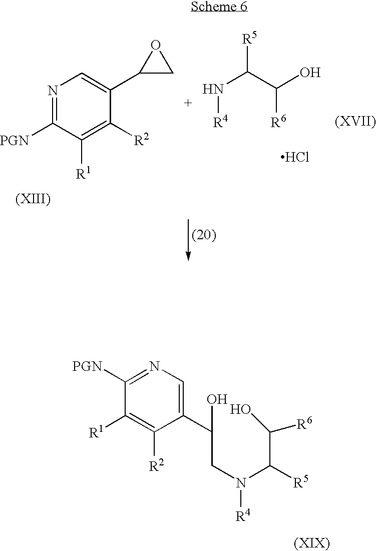

Compounds of formula (XIX) may be derived by reaction step 20—reaction of an amine of formula (XVII) with an epoxide of formula (XIII). The reaction is generally conducted in an inert solvent such as toluene or DMSO and at elevated temperature. Typical reaction conditions: involve heating (XIII) and (XVII) together in DMSO at 90° C.

An alternative method for the synthesis of compounds of formula (XVIII) is shown in Scheme 7, wherein R¹, R², R⁴, R⁵ and R⁶ are as defined above.

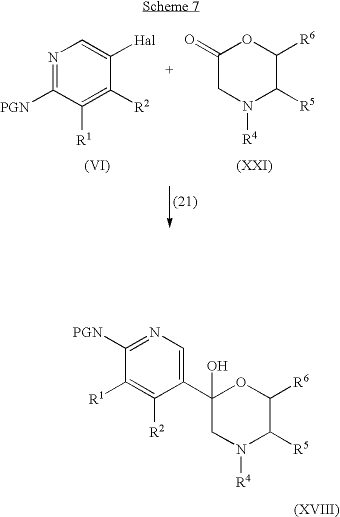

Compounds of formula (XVIII) may be prepared by reaction step 21—reaction of an organometallic reagent generated from a halogenated pyridine compound of formula (VI), with a morpholinone compound of formula (XXI) (see scheme 8). Suitable organometallic pyridine derivatives include Grignard (organomagnesium) or organolithium reagents, which may be prepared from the bromide (or iodide) by halogen-metal exchange. Typical conditions comprise addition of isopropylmagnesium chloride (or butyllithium) to the bromide (VI) in an anhydrous ethereal solvent such as tetrahydrofuran at room temperature (may require heating in certain cases when isopropylmagnesium chloride is used as the metallating agent) or below (e.g. −78° C. when butyllithium is used) to perform the halogen metal exchange reaction, followed by addition of the morpholinone (XXI) at 0° C. or lower.

Morpholinone compounds of formula (XXI), wherein R⁴, R⁵ and R⁶ are as defined above, may be prepared as shown in Scheme 8

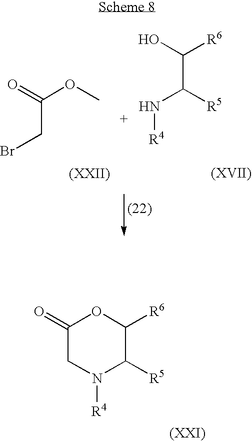

Morpholinone compounds of formula (XXI) may be prepared by reaction step 22—the reaction of an amino alcohol of formula (XVII) with an □-halo ester compound such as methyl bromoacetate (XXII) in the presence of a base such as triethylamine or 4-methylmorpholine. Typical conditions comprise 1 equivalent of the aminoalcohol (XVII) with 1-3 equivalents of triethylamine and 1 equivalent of methyl bromoacetate using toluene as solvent at room temperature or above. In some cases heating with azeotropic removal of methanol is required to achieve a good conversion to the desired product (XXI).

An alternative method for the synthesis of epoxides of formula (XIII), wherein R¹ and R² are as defined above, is shown in Scheme 9.

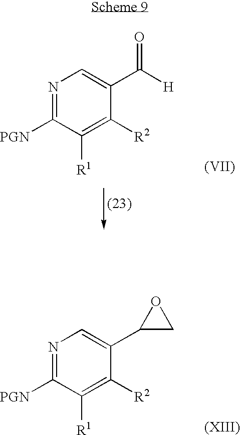

Compounds of formula (XIII) may be prepared by reaction step 23—reaction of an aldehyde of formula (VII) with a sulfur ylid reagent such as that generated from trimethylsulfonium iodide and a suitable base. Typical reaction conditions involve: reaction of trimethylsulfonium iodide (1 equivalent) with sodium hydride (1 equivalent) in DMSO at 0° C. followed by addition of the aldehyde (VII) and allowing the reaction to stand at room temperature.

A further alternative method for the synthesis of epoxide compounds of formula (XIII), wherein R¹ and R² are as defined above, is shown in Scheme 10.

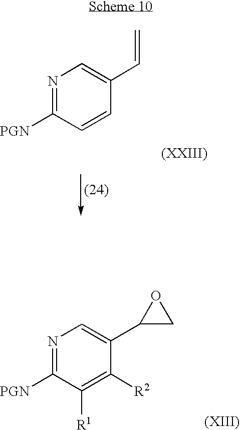

Compounds of formula (XIII) may be prepared by reaction step 24—treatment of an alkene of formula (XXIII) with an oxidising agent such as m-chloroperbenzoic acid, or dimethyldioxirane. Typical reaction conditions comprise: reaction of 1 equivalent of alkene (XXIII) with 1-2 equivalents of m-chloroperbenzoic acid in dichloromethane at room temperature.

Alkenes of formula (XXIII), wherein R¹ and R² are as defined above, may be prepared according to scheme 11.

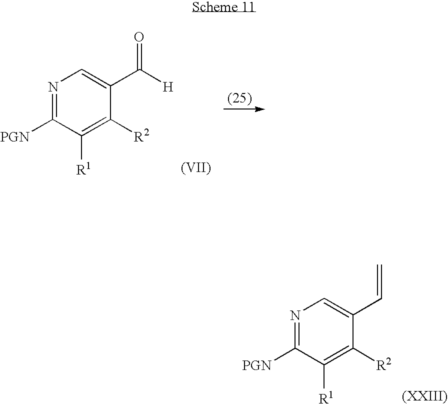

Alkene compounds of formula (XXIII) may be prepared from aldehydes of formula (VII) by reaction step 25, a Wittig or similar olefination reaction. Typical reaction conditions involved treating 1 equivalent of aldehyde (VII) with 1-2 equivalents of the ylid generated from the reaction of equal molar quantities of methyl triphenylphosphonium iodide and butyllithium, in tetrahydrofuran and room temperature or below.

Alternatively, alkenes of formula (XXIII), wherein R¹ and R² are as defined above, may be prepared according to scheme 12.

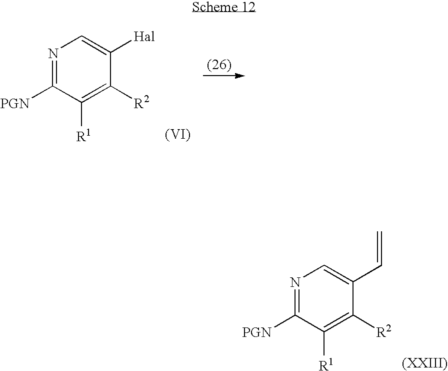

Alkene compounds of formula (XXIII) may be prepared by reaction step 26—a palladium catalysed vinylation reaction using halide compounds of formula (VI). Typical vinyl sources which may be used for this process include vinyltributylstannane, ethene gas (at high pressure), or a vinyl boronic acid. Many Pd(0) or Pd(lI) catalysts are suitable for this transformation, such as Pd(PPh₃)₄. Typical conditions comprise: reaction of a halogenated pyridine of formula (VI) (1 equivalent) with ethylene gas (at high pressure e.g. 120 psi) in an acetonitrile solution, in the presence of a Pd-catalysts such as Pd(OAc)₂ (1.5 mol %), a phosphine ligand such as tri-o-tolylphosphine (5 mol %) and amine base, such as triethylamine (large excess) at elevated temperatures (e.g. 80° C.).

In the preparation of a compound of formula (I), it will be clear to those skilled in the art that the R⁴ group (as defined above) may be introduced into any of several intermediates in the synthetic sequence. This is most conveniently achieved by reaction step 27, a reductive amination procedure. Examples of suitable intermediates for use in such a transformation are shown in Scheme 13, wherein R¹, R², R⁵ and R⁶ are as defined above. Other intermediates useful in the preparation of compounds of formula I may be equally as practicable.

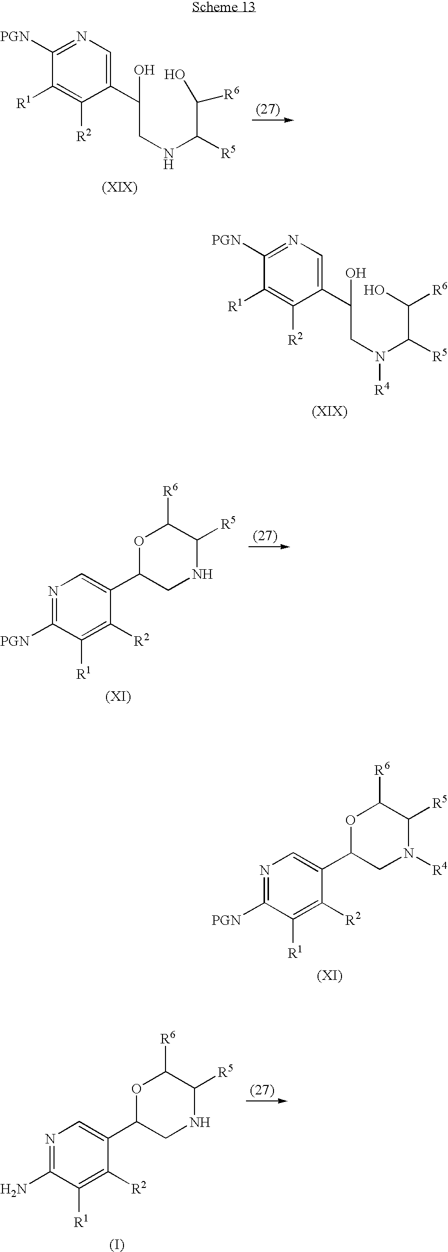

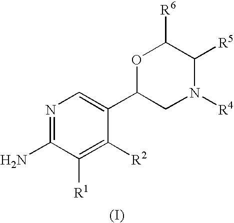

A typical procedure comprises reacting 1 equivalent of secondary amine (such as (XIX), (XI), or (I), with 1 equivalent of an aldehyde in an inert solvent such as tetrahydrofuran or dichloromethane at room temperature, then addition of 1 equivalent (or more) of sodium triacetoxyborohydride or sodium cyanoborohydride.

In some instances, for example in the preparation of compounds of formula (I) wherein R⁴ is H, R³ and A are as defined herein and wherein R¹, R², R⁵ and R⁶ are as defined above, it may be advantageous to use a protecting group PG′ prior to formation of the morpholine ring. This is illustrated in Scheme 14.

Any suitable nitrogen protecting group may be used (as described in “Protecting Groups in Organic Synthesis” 3^{rd} Edition T. W. Greene and P. G. Wuts, Wiley-Interscience, 1999). A common nitrogen protecting group (PG′) suitable for use herein is tert-butoxy carbonyl, which is readily removed by treatment with an acid such as trifluoroacetic acid or hydrogen chloride in an organic solvent such as dichloromethane. 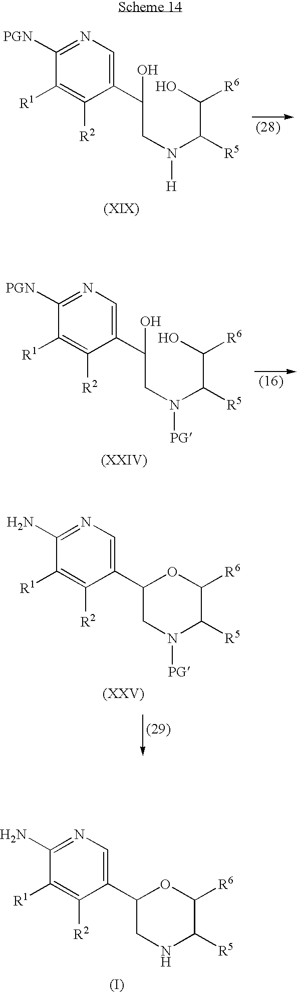
Reaction Step 28 N-Protection

A compound of formula (XXIV) may be prepared by reaction step 28—N-protection. A compound of formula (XIX) (in which R⁴═H) is reacted with a nitrogen protecting agent, such as benzyl chloroformate to produce the protected compound. Typical reaction conditions involve: reaction of 1 equivalent of secondary amine (XIX) with (1 equivalent, or more) of benzyl chloroformate in an inert solvent such as dichloromethane, together with triethylamine (1 equivalent, or more) at room temperature.

Reaction Step 16. Ring Closure

Ring closure of diol (XXIV) to morpholine (XXV) may be accomplished using many of the methods previously described herein, see Reaction Step 16, scheme 3. Particularly suitable in this instance is a Mitsunobu-type ring closure reaction through the action of a dialkyl azodicarboxylate reagent (1.1 equivalent) plus triphenylphosphine (1.1 equivalent) in an inert solvent such as tetrahydrofuran at room temperature.

Reaction Step 29 Protecting Group Removal

Compounds of formula (I) may be prepared by reaction step 29—deprotection of morpholine (XXV), under conditions dependent upon the nature of the protecting group used. For example, if benzyloxycarbonyl is used as the protecting group then it may be removed by hydrogenolysis in an inert solvent such as ethanol with a palladium catalyst such as palladium on carbon, under hydrogen pressure of 1 atmosphere or greater. If the morpholine nitrogen is protected with a benzyl group it can be deprotected by transfer hydrogenation. Typical conditions involve treating one equivalent of compound of formula (XXV) with ammonium formate (10 equivalents) in ethanol and the presence of 10% palladium on carbon as catalyst (10% by weight), at reflux for 3 hours.

Compounds of formula (XVII), wherein R⁴, R⁵ and R⁶ are as defined above, may be prepared according to scheme 15.

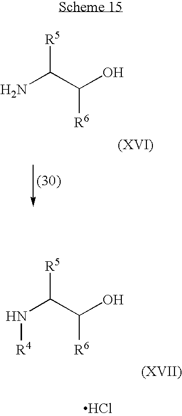

Compounds of formula (XVII) (where R⁴ is not H) may be prepared by reaction step 30, a reductive amination procedure. Typical conditions involve: reaction of 1 equivalent of amino alcohol of formula (XVI) with 1.1 equivalents of an aldehyde in dichloromethane and the presence of dried 4A molecular sieves at room temperature. After filtration and evaporation of the reaction mixture, the residue is redissolved in methanol and reacted with sodium borohydride (2 equivalents or more) at room temperature.

Alternatively the reductive amination can be accomplished in two steps via formation and then reduction of an intermediate amide, in a similar fashion to that described for Reaction Step 4 (Scheme 1) and in Reaction Steps 11 and 12 (scheme 3).

One skilled in the art will be aware that many amino alcohol compounds of formulas (XVI) and (XVII) are commercially available. Alternatively they may be prepared according to numerous methods known to those skilled in the art, such as those described in Tetrahedron 2000, 56, 2561-2576 and references cited therein.

Compounds of formula (I), wherein R¹, R², R⁴, R⁵ and R⁶ are as defined above, and R³, R⁵ and A are as described herein, may be prepared from chloropyridines of formula (XXVI) according to scheme 16.

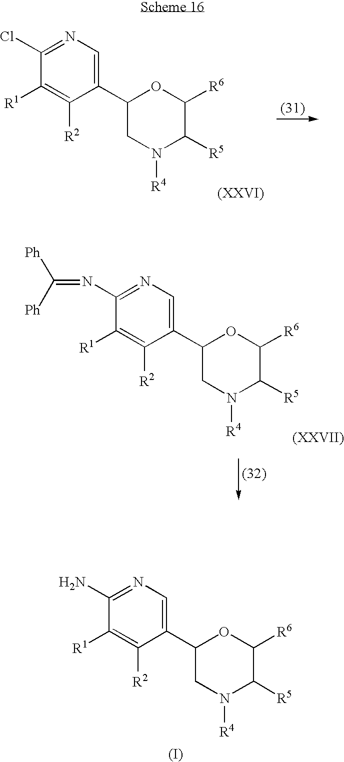

It will be clear to those skilled in the art that chloropyridine intermediates of formula (XXVI) are accessible through application of analogous synthetic methods to those previously described herein for the production of protected aminopyridine compounds of formula (XI).

Reaction Step 31. Metal Catalysed Amination Reaction

Compounds of formula (XXVII) may be prepared by reaction step 31, reaction of a compound of formula (XXVI) with benzophenone imine, in the presence of a suitable base and a metal catalyst, e.g. a Pd complex. Typical reaction conditions involve: reacting chloropyridine (XXVI) (1 equivalent) with benzophenone imine (1.2 equivalents), sodium tert-butoxide (1.4 equivalents), tris(dibenzylideneacetone)dipalladium(0) (1 mol %) and 2,2′-bis(diphenylphosphino)-1.1′-binaphthyl (BINAP) (3 mol %) in toluene at 80 to 120° C.

Reaction Step 32 Benzophenone Deprotection

Compounds of formula (XXVII) may be converted to compounds of formula (I) by hydrogenolysis (using an inert solvent and heterogeneous catalysis such as Pd on carbon at or above 1 atmosphere pressure of hydrogen), or alternatively by treatment with an aqueous acid e.g. 2M HCl in the presence of water and miscible organic solvent such as tetrahydrofuran or dioxan. Transfer hydrogenation may also be used to effect this transformation. Typical conditions involve treating one equivalent of compound of formula (XXVII) with ammonium formate (10 equivalents) in ethanol and the presence of 10% palladium on carbon as catalyst (10% by weight), at reflux for 3 hours.

Compounds of formula (I) wherein R¹, R² and R⁴ are as defined above and R³ is as defined herein, may be prepared according to scheme 17. 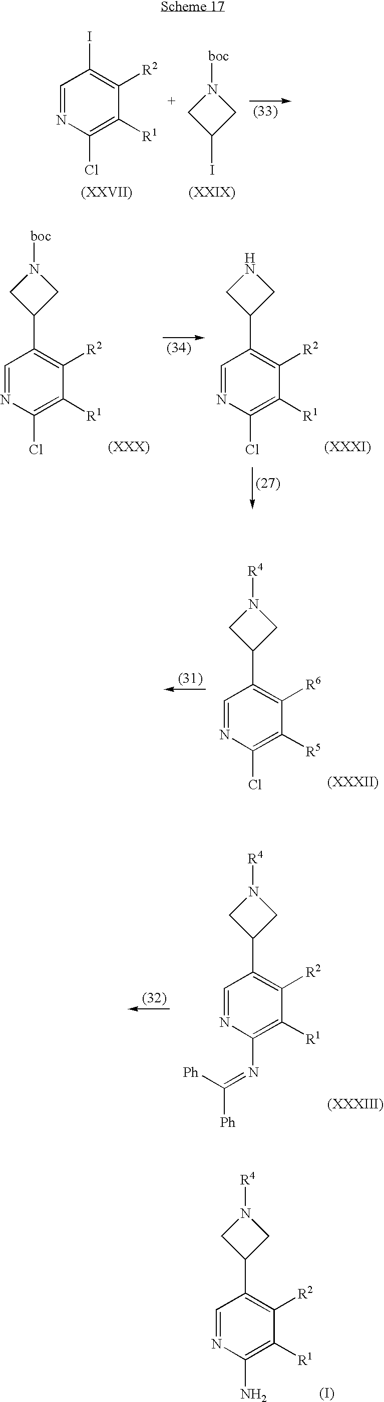
Reaction Step 33. Zincate Coupling

Compounds of formula (XXX) may be prepared by reacting compounds of the formula (XXIX) with Zn/Cu couple (or other activated Zn source) with sonication, followed by addition of a 2-chloro-4-iodopyridine and a suitable palladium catalyst and ligand, and heating to 70° C. for 18 hours. Typical conditions comprise 1 equivalent of the azetidine (XXIX) with 40 wt % Zn/Cu couple in DMF with sonication at room temperature for 4 hours, followed by addition of 1.05 equivalents of the halogenated pyridine (VI), 0.05 equivalents of tris(dibenzylideneacetone)dipalladium(0) and 0.1 equivalents of tri-o-furylphosphine and heating to 70° C. for 18 hours. Compounds of the formula (XXIX) may be prepared as described in Synlett, 4, 1998, 379.

Reaction Step 34 Boc Deprotection

Compounds of formula (XXXI) may be prepared by reacting compounds of the formula (XXX) with a suitable acid, such as HCl or TFA in a suitable solvent such as dichloromethane or diethyl ether at room temperature or above, if necessary in the presence of a cation scavenger such as Et₃SiH. Typical conditions comprise 1 equivalent of the azetidine (XXX) with CH₂Cl₂ saturated with HCl gas at 0° C. then allowing to stand at room temperature overnight.

Reaction Step 27. Reductive Amination

Compounds of formula (XXXII) may be prepared by reacting compounds of formula (XXXI) with 1-5 equivalents of the required aldehyde in a suitable solvent at room temperature in the presence of 1-5 equivalents of a suitable reducing agent such as sodium triacetoxyborohydride or sodium cyanoborohydride in a suitable solvent such as dichloromethane or tetrahydrofuran with the optional addition of acetic acid. Typical conditions comprise reacting 1 equivalent of the azetidine (XXXI) with 3.1 equivalents of the aldehyde and 3.1 equivalents of sodium triacetoxyborohydride in dichloromethane at room temperature for 18 hours.

Reaction Step 31. Metal Catalysed Amination Reaction

Compounds of formula (XXXII) may be converted to compounds of formula (I) via intermediates (XXXIII). Conversion of (XXXII) to (XXXIII) may accomplished using benzophenone imine together with a suitable base and a metal catalyst, e.g. a Pd(0) complex. Typical reaction conditions involve: reacting chloropyridine (XXXII) (1 equivalent) with benzophenone imine (1.2 equivalents), sodium tert-butoxide (1.4 equivalents), tris(dibenzylideneacetone)dipalladium(0) (1 mol %) and 2,2′-bis(diphenylphosphino)-1.1′-binaphthyl (BINAP) (3 mol %) in toluene at 80 to 120° C.

Reaction Step 32. Benzophenone Deprotection

Compounds of formula (XXXIII) may be converted to compounds of formula (I) either by hydrogenolysis (using an inert solvent and heterogeneous catalysis such as Pd on carbon at or above 1 atmosphere pressure of hydrogen), or by treatment with an aqueous acid in the presence of a water miscible organic solvent such as tetrahydrofuran or dioxan. Transfer hydrogenation may also be used for effect this transformation. Typical conditions involve treating one equivalent of compound of formula (XXVII) with ammonium formate (10 equivalents) in ethanol and the presence of 10% palladium on carbon as catalyst (10% by weight), at reflux for 3 hours.

Alternatively, compounds of formula (I), wherein R¹, R² and R⁴ are as defined above and R³ is as defined herein, may be prepared according to scheme 18. 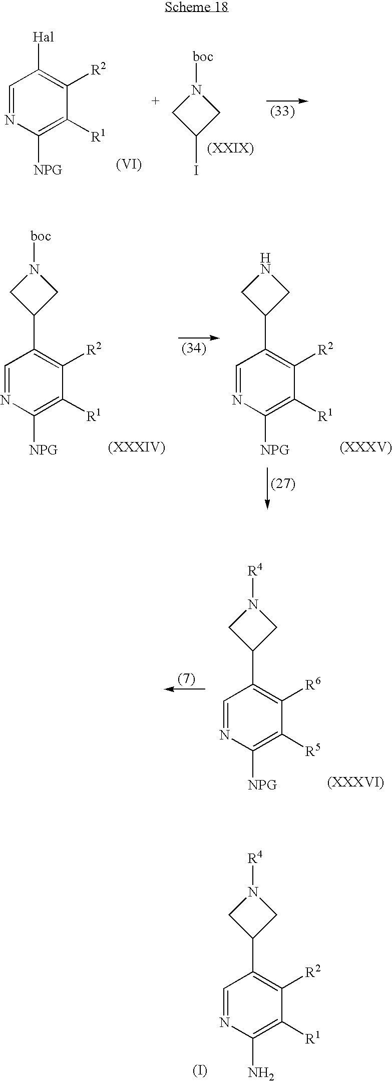
Reaction Step 33. Zincate Coupling

Compounds of formula (XXXIV) may be prepared by reacting compounds of the formula (XXIX) with Zn/Cu couple (or other activated Zn source) with sonication, followed by addition of compounds of the formula (VI) and a suitable palladium catalyst and ligand, and heating to 70° C. for 18 hours. Typical conditions comprise 1 equivalent of the azetidine (XXIX) with 40 wt % Zn/Cu couple in DMF with sonication at room temperature for 4 hours, followed by addition of 1.05 equivalents of the halogenated pyridine (VI), 0.05 equivalents of tris(dibenzylideneacetone)dipalladium(0) and 0.1 equivalents of tri-o-furylphosphine and heating to 70° C. for 18 hours. Compounds of the formula (XXIX) may be prepared as described in Synlett, 4, 1998, 379.

Reaction Step 34. N-Boc Deprotection

Compounds of formula (XXXV) may be prepared by reacting compounds of the formula (XXXIV) with a suitable acid, such as HCl or TFA in a suitable solvent such as dichloromethane or diethyl ether at room temperature or above, if necessary in the presence of a cation scavenger such as Et₃SiH Typical conditions comprise 1 equivalent of the azetidine (XXXIV) with CH₂Cl₂ saturated with HCl gas at 0° C. then allowing to stand at room temperature overnight.

Reaction Step 27. Reductive Amination

Compounds of formula (XXXVI) may be prepared by reacting compounds of formula (XXXV) with 1-5 equivalents of the required aldehyde in a suitable solvent at room temperature in the presence of 1-5 equivalents of a suitable reducing agent such as sodium triacetoxyborohydride or sodium cyanoborohydride in a suitable solvent such as dichloromethane or tetrahydrofuran with the optional addition of acetic acid. Typical conditions comprise 1 equivalent of the azetidine (XXXV) with 3.1 equivalents of the aldehyde and 3.1 equivalents of sodium triacetoxyborohydride in dichloromethane at room temperature for 18 hours.

Reaction Step 7. Aminopyridine Deprotection

Compounds of formula (XXXVI) may be converted to compounds of formula (I) by deprotection. The nature of this reaction will depend upon the protecting group selected for use. For example, when the 2,5-dimethylpyrrole system is used to protect the aminopyridine group it may be deprotected by treatment with hydroxylamine. Typical conditions comprise 1.0 equivalents of compound (XXXVI) and 5 equivalents of hydroxylamine hydrochloride in ethanol at reflux.

Schemes 19-29

Compounds of formula I wherein R³ is moiety IV and unless otherwise indicated R¹, R², R⁸, R⁹ and R¹⁰ are as defined above, may be prepared using methods described in Schemes 19-29. 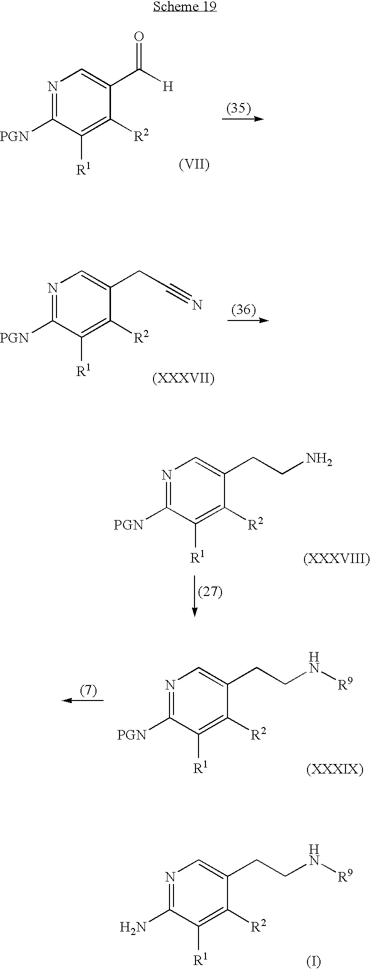
Reaction Step 35. Nitrile Formation

Compounds of formula (VII) may be converted to nitrile compounds of formula (XXXVII) by reaction with tosylmethyl isocyanide (TosMic). Typical conditions involve: treating aldehyde (VII) (1 equivalent) with TosMic (1 equivalent) and potassium tert-butoxide (2 equivalents) in ethylene glycol dimethyl ether at 45° C. after a period of 30 minutes methanol is added and the reaction mixture allowed to reach room temperature.

Reaction Step 36. Nitrile Reduction

Nitriles of formula (XXXVII) may be converted to amines of formula (XXXVIII) by reduction of the nitrile group. This reduction may be achieved through the action of a hydride reducing agent, such as lithium aluminium hydride, or sodium borohydride in the presence of a transition metal salt, such as NiCl₂ or CoCl₂. Alternatively the nitrile group may be reduced by hydrogenation with a transition metal catalyst such as Raney Nickel or Pd on carbon.

Typical conditions involve: reacting nitrile (XXXVII) (1 equivalent) with nickel chloride (1 equivalent) in methanol followed by cautious addition of sodium borohydride (3 equivalents or more) at 0° C.

Reaction Step 27. Reductive Amination.

The primary amines (XXXVIII) may be converted to compounds of formula (XXXIX) by a reductive amination procedure, by reaction with an aldehyde and hydride reducing agent such as sodium triacetoxyborohydride or sodium borohydride Typical conditions involve: reacting compounds of formula (XXXVIII) with a suitable aldehyde (1 equivalent or more) in the presence of a hydride reducing agent such as sodium cyanoborohydride or sodium triacetoxyborohydride (1 equivalent or more) in an alcoholic solvent such as ethanol.

Reaction Step 7 Aminopyridine Deprotection

Compounds of formula (XXXIX) may be converted to compounds of formula I by using a reaction to deprotect the nitrogen of the aminopyridine group (PGN) to liberate the NH₂ in compounds (I). The nature of this reaction will depend upon the protecting group selected for use. For example, when the 2,5-dimethylpyrrole system is used to protect the aminopyridine group, it may be deprotected by treatment with hydroxylamine. Typical conditions comprise 1.0 equivalents of compound (XXXIX) and 5 equivalents of hydroxylamine hydrochloride in ethanol at reflux.

Alternatively, nitrile compounds of formula (XXXVII) may be converted into compounds of formula (I) as shown in Scheme 20. 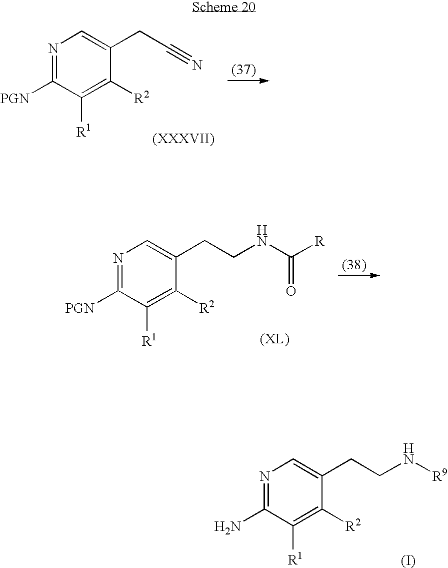
Reaction Step 37. Reductive Acylation

The intermediates of formula (XXXVII) may be reduced e.g. with sodium borohydride and nickel chloride in the presence of an acylating agent, such as a carboxylic acid anhydride to afford amide intermediates of formula (XL). Typical conditions involve: reacting nitrile (XXXVII) (1 equivalent) with nickel chloride (1 equivalent) and a carboxylic acid anhydride (1 equivalents or more) in methanol followed by cautious addition of sodium borohydride (3 equivalents or more) at 0° C.

Reaction Step 38. Amide Reduction

Amides of general formula (XL) may be reduced to amines using borane or lithium aluminium hydride. Typical conditions comprise 1.0 equivalents of amide (XL), 1.2-3.0 equivalents of borane in THF at reflux followed by heating in strong aqueous acid, such as 5M HCl. The resulting amine intermediates may then be deprotected to give the aminopyridine compounds of formula (I), as previously described in Reaction Step 7.

An alternative preparation of nitrile compounds of formula (XXXVII) is shown in Scheme 21.

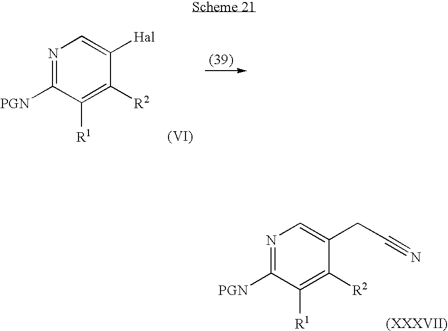

Compounds of formula (XXXVII) may be prepared by reaction step 39—reaction of halogenated pyridine (VI) with tributyl(cyanomethyl)stannane and a palladium catalyst according to the procedure described in Chem. Lett 1984, 1511-1512. Typical conditions involve treating 1 equivalent of (VI) with 1.5 equivalents of tributyl(cyanomethyl)stannane, bis(acetonitrile)dichloropalladium(II) (2.5 mol %) and tri-otolylphosphine (5 mol %) in xylene at 120° C.

An alternative procedure for the preparation of compounds of formula (XXXVIII) is shown in scheme 22. 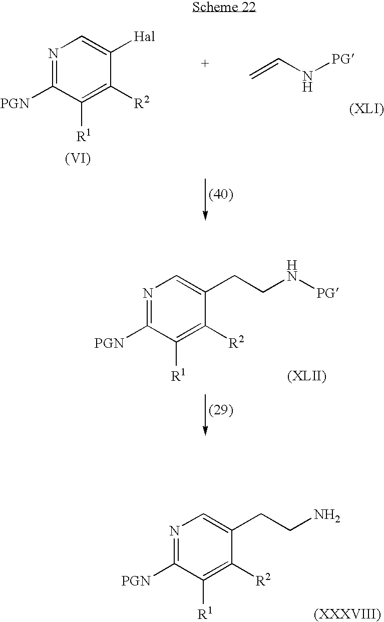
Reaction Step 40. B-alkyl Suzuki Coupling

Protected amides of formula (XLII) are available by B-alkyl Suzuki coupling between a vinyl carbamate (XLI) and a halogenated pyridine (VI), in a similar fashion to that described in J. Org. Chem. 1999, 64, 8743-8744. In a typical procedure benzyl vinyl carbamate [commercially available, or prepared as described in J. Org. Chem. 1999, 64, 8743-8744] was treated with 1 equivalent of 9BBN solution in tetrahydrofuran at −10° C. After completion of the hydroboration stage, the resulting organoboron intermediate is treated with sodium hydroxide, PdCl₂(dppf).CH₂Cl₂ complex is added together with a halogenated pyridine of formula (VI).

Reaction Step 29 Amine Deprotection

Compounds of formula (XLII) may be converted to compounds of formula (XXXVIII) by deprotection. The nature of this reaction will depend upon the protecting group selected for use. For example, when benzyloxycarbonyl is used as the protecting group then it may be removed by hydrogenolysis in an inert solvent such as ethanol with a palladium catalyst such as palladium on carbon. Typical reaction conditions involve: reacting compounds of formula (XLII) in an alcohol solvent (such as ethanol) with hydrogen (at a pressure of 1 atmosphere of greater) in the presence of a transition metal catalyst such as Pd on carbon.

An alternative method for the production of compounds of formula (XXXIX) is shown in Scheme 23.

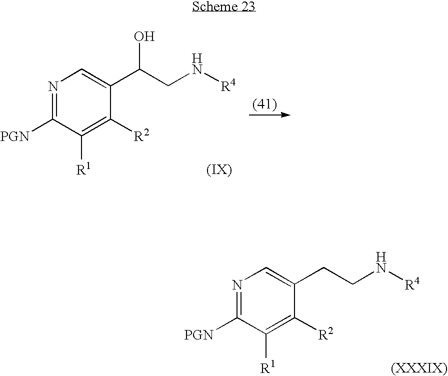

Compounds of formula (XXXIX) may be prepared by reaction step 41—deoxygenation of a compound of formula (IX) by, for example, hydrogenation (in an inert solvent such as ethanol, in the presence of a transition metal catalyst, such as Pd on carbon in an atmosphere of hydrogen (1 atmosphere or higher)). Alternatively, a hydride source such as triethylsilane in conjunction with a suitable acid may be used (as described in Heterocycles 2003, 1203-1209) Typical reaction conditions involve: dissolving (IX) in a mixture dichloromethane and trifluoroacetic acid at room temperature and adding 1 (or more) equivalents of triethylsilane.

A further method for the production of compounds of formula (XXXVII) is shown in scheme 24. 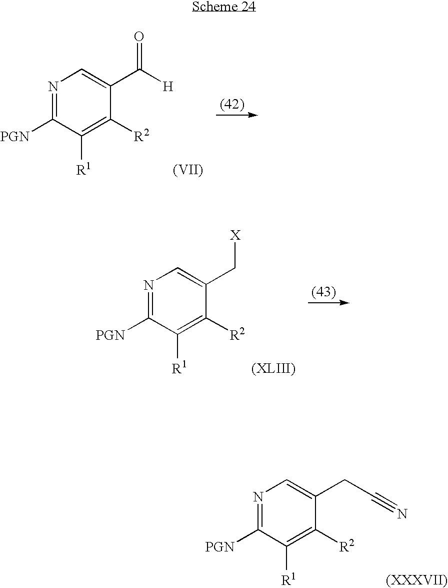
Reaction Step 42. Conversion to Benzylic Electrophile.

An aldehyde of formula (VI I) is reduced by treatment with a hydride reducing agent such as sodium borohydride in an alcoholic solvent, such as ethanol at room temperature. The resulting alcohol can be activated towards nucleophilic displacement by conversion to a group X (generally a halide or sulfonate ester) to give intermediates of formula (XLIII). Typical conditions involve: reaction of one equivalent of methanesulfonyl chloride and one equivalent of an amine base such as triethylamine in an inert solvent such as dichloromethane at 0° C.

Reaction Step 43. Cyanide Displacement

Intermediates of formula (XLIII) can be converted to compounds of formula (XXXVII) by the action of a nucleophilic source of cyanide, such as KCN, in an inert solvent, such as dimethylformamide, at or above room temperature, in a procedure analogous to that described in U.S. Pat. No. 5,914,319.

A further method for the production of compounds of formula (XXXIX) is shown in Scheme 25. 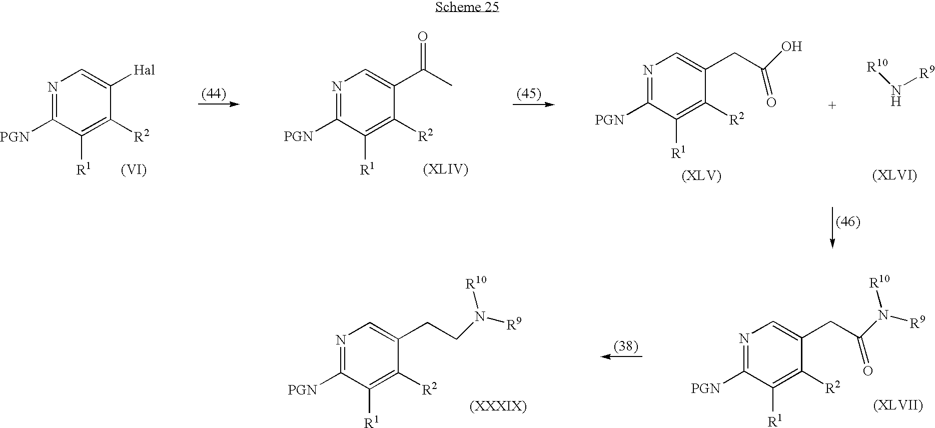
Reaction Step 44. Methyl Ketone Formation

Halogenated pyridines or formula (VI) can be converted to methyl ketones of formula (XLIV) by treatment first with butyllithium (or other agent capable of facilitating a halogen metal exchange reaction) and treating the resultant organometallic intermediate with a suitable acetyl source, such as acetylmorpholine or the Weinreb amide derived from acetic acid.

Reaction Step 45. Willgerodt-Kindler Reaction

Methyl ketones of formula (XLIV) may be converted into arylacetic acids of formula (XLV) by treatment with sulfur and morpholine. A typical procedure involves: reacting 1 equivalent of methyl ketone (XLIV) with sulfur (2 equivalents) and in excess morpholine at reflux (either neat or in an alcoholic solvent such as ethanol), followed by hydrolysis either in refluxing 2M hydrochloric acid or 2M NaOH.

Reaction Step 46. Amide Formation.

Pyridyl acetic acids of formula (XLV) may be converted to amides of formula (XLVI) by reaction with an amine of formula (XLVII) and a suitable amide coupling reaction, such as by reaction with an acid chloride or anhydride then addition of a suitable amine, or using a peptide coupling reagent such dicylcohexyl carbodiimide. or other carbodiimide reagent. For example, acid chlorides in the presence of a suitable base such as triethylamine or 4-methylmorpholine may be used for the amide forming stage. Typical reaction conditions comprise conversion of the acid (XLV) to the acid chloride by treatment with oxalyl chloride with a trace of dimethylformamide as catalyst in an inert solvent such as dichloromethane. After evaporation of solvents and excess oxalyl chloride, 1.0 equivalents of amine (XLVII), 1.2-2.0 equivalents of base (preferably triethylamine) are reacted with 1.0 equivalents of the acid chloride in dichloromethane at 25° C.

Reaction Step 38 Amide Reduction

Amides of general formula (XLVI) may be converted into compounds of formula (XXXIX) by reduction with a hydride reducing agent, such as borane-tetrahyrdrofuran complex. Typical conditions comprise 1.0 equivalents of amide (XLVI), 1.2-3.0 equivalents of borane in THF at reflux then treatment with a strong acid such as 5M HCl at elevated temperature to hydrolyse the initially formed boron complexes with the product. Other non-acidic methods are available for breaking the boron complex e.g. treatment with diethanolamine.

An alternative method for the production of compounds of formula (XLV) is shown Scheme 26.

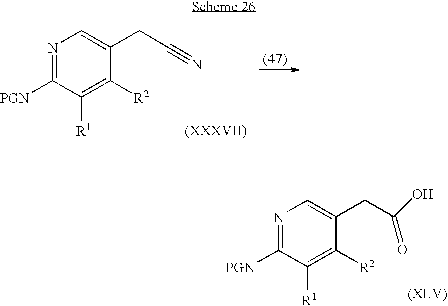

Compouns of formula (XLV) may be prepared according to reaction step 47, hydrolysis. A nitrile of general formula (XXXVII) is hydrolysed by heating in a strongly acidic or basic aqueous solution. Typical conditions involve heating a compound of formula (XXXVII) in a 5M HCl solution at reflux.

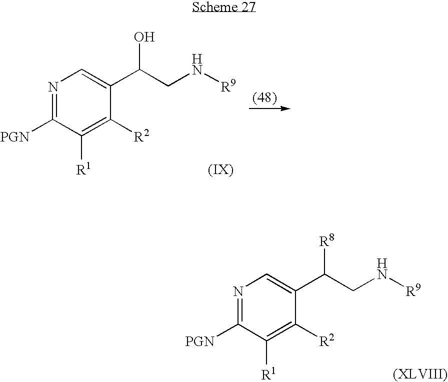

Compounds of formula (XLVIII), wherein R⁸ is OMe, can be formed from compounds of formula (IX) by reaction step 48—methylation of alcohol (IX) with a suitable electrophilic methyl source, such as iodomethane. In general, a strong base, such as sodium hydride is also required. Typical conditions involve treating 1 equivalent of (IX) with 1.1 (or more) of sodium hydride in an inert solvent such as tetrahydrofuran or dimethylformamide then adding 1 (or more) equivalents of iodomethane at room temperature.

Compounds of general formula (XLVIII) can subsequently be converted into compounds of formula (I) using the same methods as described for the conversion of compounds of formula (XXXIX), as shown in Scheme 19.

Further examples of compounds of formula (I), wherein R⁸ is not hydrogen, can be prepared according to scheme 28. 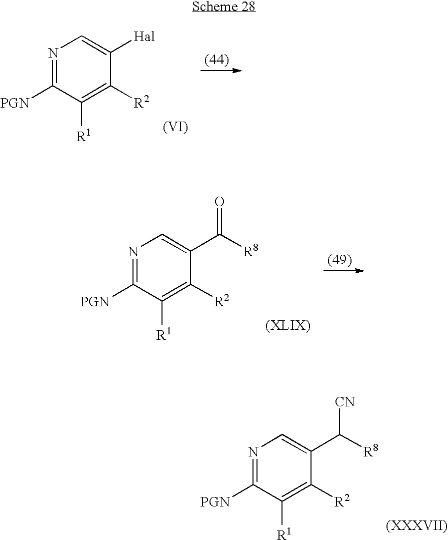
Reaction Step 44. Ketone Formation

Halogenated pyridine compounds of formula (VI) can be readily converted to ketones of formula (XLIX) using methods similar to the formation of methyl ketone compounds of formula (XLIV) (scheme 25). Namely, by treatment first with butyllithium (or other agent capable of facilitating a halogen metal exchange reaction) and treating the resultant organometallic intermediate with a suitable acyl source, such as acylmorpholine or Weinreb amide (both which are readily prepared using methods well-known to the skilled person).

Reaction Step 45. Nitrile Formation

The ketone of formula (XLIX) can then be converted to nitrile (XXXVII) by reaction with tosylmethyl isocyanide (TosMic). Typical conditions involve: treating ketone (XLIX) (1 equivalent) with TosMic (1 equivalent) and potassium tert-butoxide (2 equivalents) in ethylene glycol dimethyl ether at −45° C. After a period of 30 minutes methanol is added and the reaction mixture allowed to reach room temperature.

Nitriles of formula (XXXVII) may subsequently be converted to compounds of formula (I) using the procedures previously described in scheme 19.

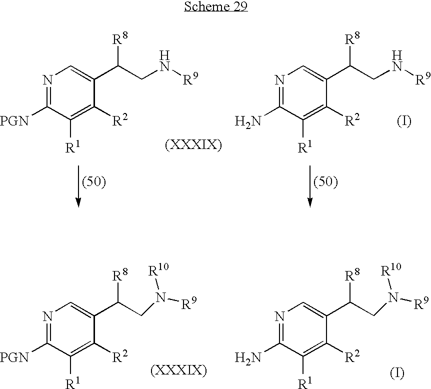

Compounds of formula (XXXIX) or (I) wherein R¹⁰=H, may be readily converted into further compounds of formula (XXXIX) or (I) wherein R¹⁰ is not H, by reaction step 50—a reductive amination procedure as shown in Scheme 29. A typical Procedure involves reacting 1 equivalent of a secondary amine (such as (XXXIX) or (I)), with 1 equivalent of an aldehyde in an inert solvent such as tetrahydrofuran or dichloromethane at room temperature, then addition of 1 equivalent (or more) of sodium triacetoxyborohydride or sodium cyanoborohydride.

Alternatively, the reductive amination may be conducted in two steps via an intermediate amide in similar fashion to that described for Reaction Step 4 in scheme 1.

Compounds of formula (I) wherein R¹, R², R⁴ and R⁵ are as defined above, and R³, R⁵ and A are as described herein, may be prepared according to reaction scheme 30. 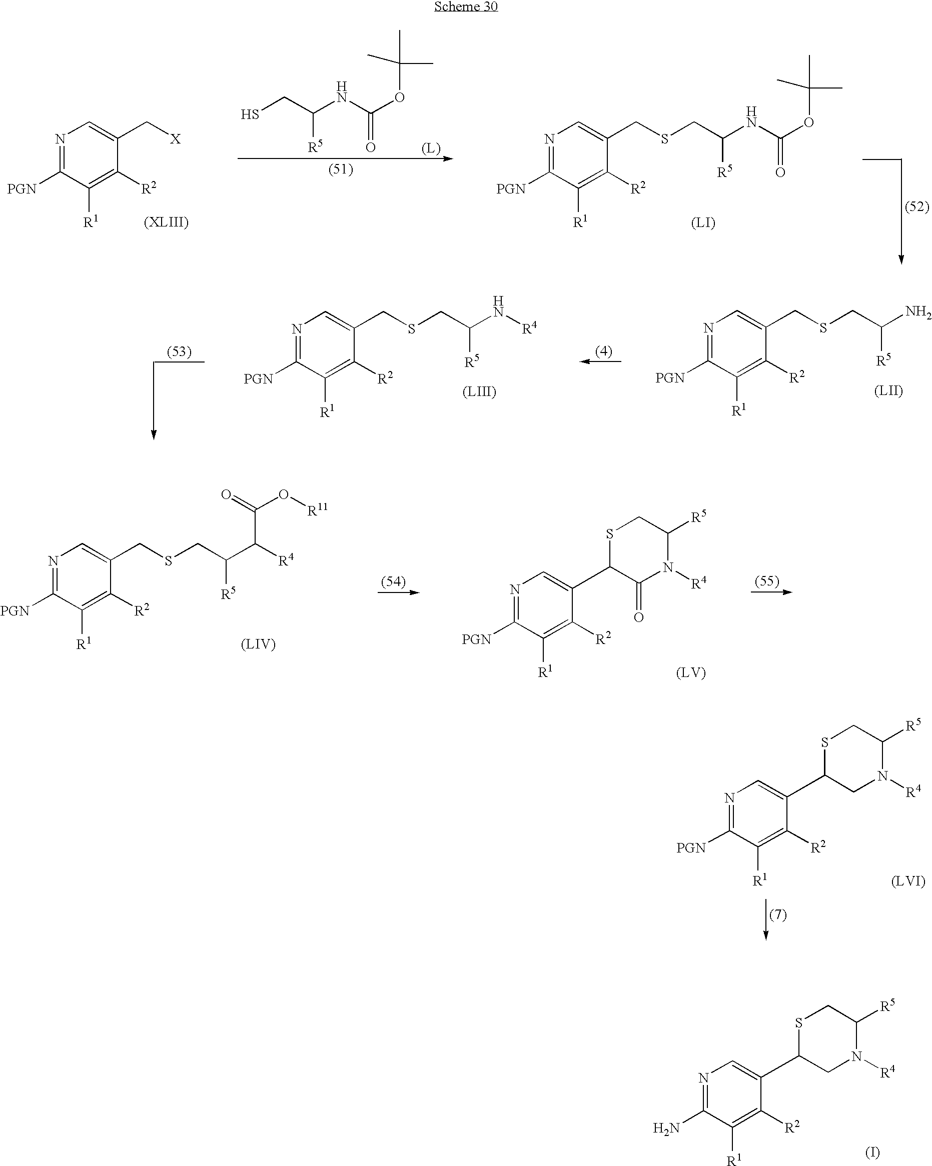
Reaction Step 51. Thioether Formation

Thioethers of the formula (LI) may be formed by reaction of a compound of formula (XLIII), wherein X is generally a halide or a sulfonate ester, with a compound of the formula (L) [commercially available or prepared a described in J. Chem. Soc Perkin I, 1987, 111-120] in the presence of a base in an alcoholic solvent.

Typical conditions comprise 1.0 equivalents of alkylhalide, 1.0 equivalents of thiol and 1.0-4.0 equivalents of a tertiary amine base such as triethylamine in an alcoholic solvent such as ethanol.

Reaction Step 52. N-Boc Deprotection

Compounds of formula (LII) may be prepared by reacting compounds of the formula (LI) with a suitable acid, such as HCl or TFA in a suitable solvent such as dichloromethane or diethyl ether at room temperature or above, if necessary in the presence of a cation scavenger such as Et₃SiH Typical conditions comprise adding 1 equivalent of the protected amine (LI) to CH₂Cl₂ saturated with HCl gas at 0° C. then allowing to stand at room temperature overnight.

Reaction Step 4. Reductive Amination

Compounds of formula (LIII) may be prepared from compounds of formula (LII) by employing standard amide bond forming conditions followed by reduction of the intermediate amide with a hydride reducing agent such as borane or lithium aluminium hydride.

For example, acid chlorides in the presence of a suitable base such as triethylamine or 4-methylmorpholine may be used for the amide forming stage. Typical reaction conditions comprise 1.0 equivalents of amine (LII), 1.2-2.0 equivalents of base (preferably triethylamine), 1.1-1.3 equivalents of acid chloride in dichloromethane at 25° C. Reducing agents such as borane or lithium aluminium hydride can be used for the amide reduction stage. Typical conditions comprise 1.0 equivalents of amide, 1.2-3.0 equivalents of borane in THF at reflux, followed by treatment with a strong acid to hydrolyse the initially formed boron complex of the product. Other non-acidic methods are available for breaking the boron complex e.g. treatment with diethanolamine.

Reaction Step 53. Carbamate Formation

Compounds of formula (LIV), wherein R¹¹ is benzyl or (C₁-C₆)alkyl, may be formed by treatment of compounds of formula (LIII) with an alkyl or benzyl chloroformate in an inert solvent such as dichloromethane or diethyl ether in the presence of a base. Typical conditions comprise 1.0 equivalents of the amine (LIII), 1.0 equivalents of an alkylchloroformate such as methylchloroformate and 1.0-3.0 equivalents of a tertiary amine base such as triethylamine in diethyl ether at 25° C.

Reaction Step 54. Thiomorpholinone Ring Formation

Compounds of the formula (LV) may be formed by treatment of thioether (LIV) with a strong base such as lithium diisopropylamide in an inert solvent such as diethyl ether or THF.

Typical conditions comprise addition of 3.0 equivalents of a strong base such as lithium diisopropylamide to 1.0 equivalents of the thioether (LIV) at a temperature below −50° C. in an inert solvent such as THF and allowing to warm to ambient temperature.

Reaction Step 55. Amide Reduction

Compounds of formula (LVI) may be prepared by reaction of compounds of formula (LV) with reducing agents such as borane or lithium aluminium hydride. Typical conditions comprise 1.0 equivalents of amide (LV), 1.2-3.0 equivalents of borane in THF at reflux, followed by treatment with a strong acid to hydrolyse the initially formed boron complex. Other non-acidic methods are available for breaking the boron complex e.g. treatment with diethanolamine.

Reaction Step 7. Aminopyridine Deprotection

Compounds of formula (LVI) may be converted to compounds of formula (I) by deprotection. The nature of this reaction will depend upon the protecting group selected for use.

For example, when the 2,5-dimethylpyrrole system is used to protect the aminopyridine group it may be deprotected by treatment with hydroxylamine. Typical conditions comprise 1.0 equivalents of compound (LVI) and 5 equivalents of hydroxylamine hydrochloride in ethanol at reflux.

Compounds of formula (LIX) wherein R¹, R² and R⁵ are as defined above, may be prepared according to reaction scheme 31. 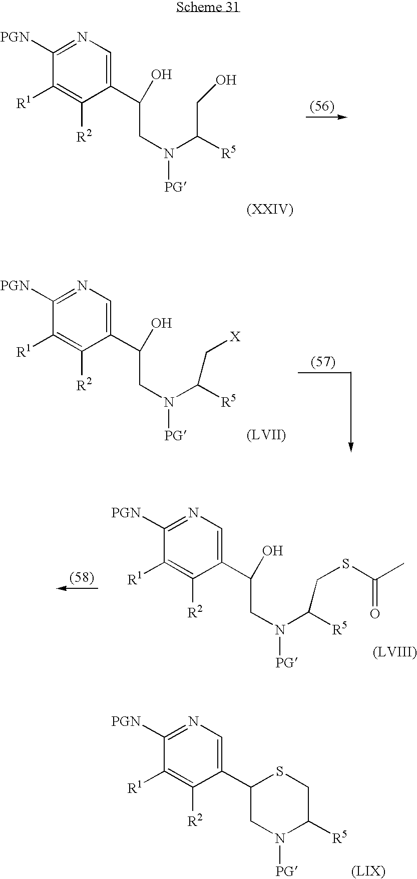
Reaction Step 56. Primary Alcohol Activation

Compounds of the formula (LVII) may be formed from compounds of the formula (XXIV), wherein PG′ is a carbamate protecting group such as tert-butyloxycarbonyl or benzyloxycarbonyl, by selective conversion of the primary hydroxyl group to a group X (generally a halide or sulfonate ester). Typical conditions involve: reaction of one equivalent of toluenenesulfonyl chloride and one equivalent of an amine base such as triethylamine in an inert solvent such as dichloromethane at 0° C.

Reaction Step 57. Thioacetate Formation

Compounds of the formula (LVIII) may be formed by from compounds of the formula (LVII), wherein PG′ is a carbamate protecting group such as tert-butyloxycarbonyl or benzyloxycarbonyl, by reaction with a suitable nucleophile such as thioacetic acid in an inert solvent such as acetonitrile in the presence of a suitable base. Typical conditions involve: reaction of one equivalent of compounds of the formula (LVII) with 1.0-2.0 equivalents of thioacetic acid in the presence of 1.0-5.0 equivalents of a suitable base such as potassium carbonate in an inert solvent such as acetonitrile and the mixture heated to reflux.

Reaction Step 58. Ring Closure

Compounds of the formula (LVIII) may be activated towards nucleophilic displacement at the benzylic centre by conversion to a group X (generally a halide or sulfonate ester). In situ ring closure may then occur to provide compounds of the formula (LIX). Typical conditions involve: reaction of one equivalent of toluenenesulfonyl chloride and one equivalent of an amine base such as triethylamine in an inert solvent such as dichloromethane at 0° C. Evaporation of the solvent followed by redissolution in a higher boiling solvent such as acetonitrile with 0-5.0 equivalents of a suitable base such as potassium carbonate and heating of the mixture to reflux may be necessary to effect the ring closure.

Compounds of the formula (LIX), wherein PG′ is a carbamate protecting group such as tert-butyloxycarbonyl or benzyloxycarbonyl, may be converted to compounds of the formula (I) using procedure analogous to those described in Scheme 18 for the conversion of compounds of formula (XXXIV) into compounds of formula (I).

Compounds of formula (I) wherein R¹, R² and R⁴ are as defined above, and R³ is as described herein, may be prepared according to reaction scheme 32. 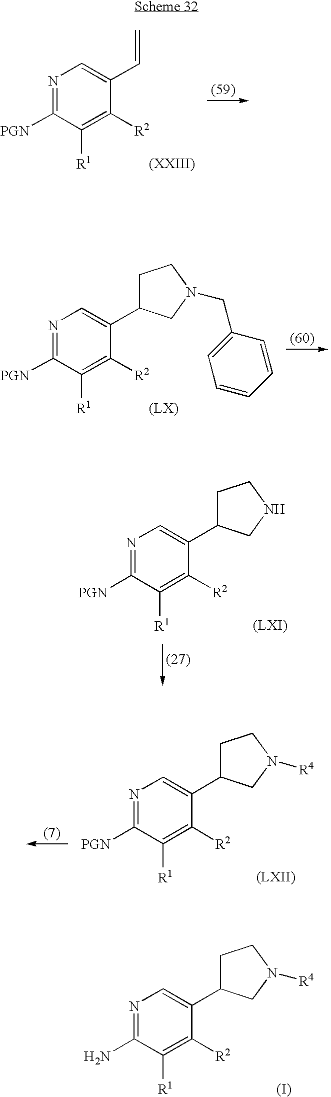
Reaction Step 59. Cycloaddition

Compounds of the formula (LX) may be formed from an alkene of the formula (XXIII) by reacting with N-benzyl-N-(methoxymethyl)-trimethylsilylmethylamine and a catalytic amount of an acid such as trifluoroacetic acid in an inert solvent such as dichloromethane, acetonitrile, tetrahydrofuran or toluene at −10° C. to the reflux temperature of the reaction mixture. Alternative catalysts include anhydrous potassium or cesium fluoride, tetra-n-butylammonium fluoride, trifluoromethanesulfonic acid, trimethylsilyltrifluoromethanesulfonate and iodotrimethylsilane. Typical conditions involve: reaction of 1 equivalent of alkene (XXIII) with 1.5 equivalents of N-benzyl-N-(methoxymethyl)-trimethylsilylmethylamine and 0.1 equivalents of trifluoroacetic acid in dichloromethane.

Reaction Step 60. Pyrrolidine Debenzylation

Compounds of the formula (LX) may be deprotected to secondary amines of the formula (LXI) by hydrogenolysis in an inert solvent such as ethanol with a palladium catalyst such as palladium on carbon, under hydrogen pressure of 1 atmosphere or greater. Alternatively it can be deprotected by transfer hydrogenation. Typical conditions involve treating one equivalent of compound of formula (LX) with ammonium formate (10 equivalents) in ethanol and the presence of 10% palladium on carbon as catalyst (10% by weight), at reflux for 3 hours.

Reaction Step 27. Reductive Amination

Compounds of formula (LXII) may be prepared by reacting compounds of formula (LXI) with 1-5 equivalents of the required aldehyde in a suitable solvent at room temperature in the presence of 1-5 equivalents of a suitable reducing agent such as sodium triacetoxyborohydride or sodium cyanoborohydride in a suitable solvent such as dichloromethane or tetrahydrofuran with the optional addition of acetic acid. Typical conditions comprise reacting 1 equivalent of the pyrrolidine (LX) with 3.1 equivalents of the aldehyde and 3.1 equivalents of sodium triacetoxyborohydride in dichloromethane at room temperature for 18 hours.

Reaction Step 7. Aminopyridine Deprotection

Compounds of formula (LXI) may be converted to compounds of formula (I) by deprotection. The nature of this reaction will depend upon the protecting group selected for use.

For example, when the 2,5-dimethylpyrrole system is used to protect the aminopyridine group it may be deprotected by treatment with hydroxylamine. Typical conditions comprise 1.0 equivalents of compound (LXI) and 5 equivalents of hydroxylamine hydrochloride in ethanol at reflux.

Compounds of formula (I) wherein R¹, R², R⁴, R⁵ and R⁶ are as defined above and R³ and A are as described herein, may be prepared according to reaction scheme 33. 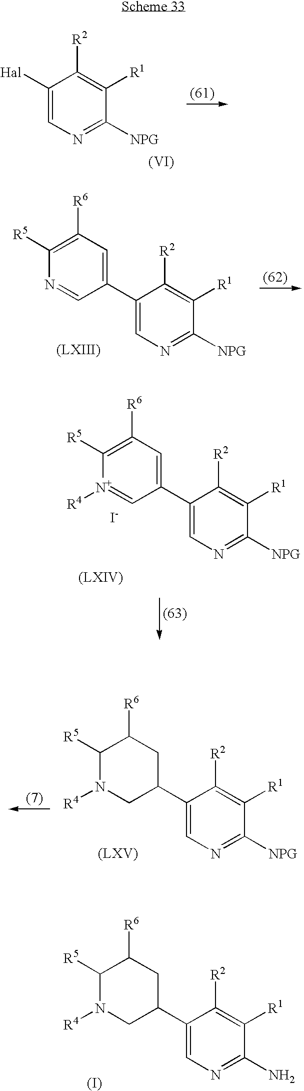
Reaction Step 61. Reaction with 3-pyridyl Borane

Compounds of formula (LXIII) may be prepared from compounds of formula (VI) by reaction with 3-pyridyl boranes (or similar boronic acid) in the presence of a suitable base and suitable palladium catalyst. Typical conditions comprise addition of the 3-pyridyl borane to a compound of formula (VI) in toluene/ethanol as solvent, in the presence of tetrakis(triphenylphosphine)palladium(0) and sodium carbonate, followed by heating to reflux. Examples of 3-pyridyl boranes (or similar boronic acids) are commercially available.

Reaction Step 62. Alkylation

Compounds of formula (LXIV) may be prepared from compounds of formula (LXIII) by addition of an alkyl iodide. Typical conditions comprise addition of the alkyl iodide to a compound of formula (LXIII), in a suitable solvent such as acetonitrile and then heating to reflux.

Reaction Step 63. Hydrogenation

Compounds of formula (LXV) may be prepared from compounds of formula (LXIV) by hydrogenation. Typical conditions comprise hydrogenation of a compound of formula (LXIV), at elevated pressure, in a suitable solvent such as ethanol, in the presence of a suitable catalyst such as PtO₂.

Reaction Step 7. Aminopyridine Deprotection

Compounds of formula (LXV) may be converted to compounds of formula (I) by deprotection. The nature of this reaction will depend upon the protecting group selected for use.

For example, when the 2,5-dimethylpyrrole system is used to protect the aminopyridine group it may be deprotected by treatment with hydroxylamine. Typical conditions comprise 1.0 equivalents of compound (LXV) and 5 equivalents of hydroxylamine hydrochloride in ethanol at reflux.

Methods for Resolution of Racemic Compound

In cases where the above methods lead to racemic products, many methods are available for the separation of the racemate into its constituent enantiomers. These include:
(1) formation and selective crystallisation of diastereomeric salts produced by salt formation between a racemic base and an enantiomerically pure chiral acid component (or vice versa)
(2) HPLC using a chiral stationary phase—many of which are commercially available
(3) Formation of diastereomeric adducts by reaction of a racemic compound with an enantiomerically pure chiral compound or reagent, subsequent separation of the constituent diastereoisomers by physical methods, including crystallisation or chromatography, and splitting of the separated adducts to release the desired compound in enantiomerically enriched form. This is often termed a classical resolution. For example, a racemic alcohol may be reacted with an enantiomerically pure chiral acid to form diastereomeric esters using standard ester forming reactions. These esters can then be separated e.g. by selective crystallisation. The separated diastereomeric esters may then separately be hydrolysed under standard ester hydrolysis conditions to release chiral alcohols in enantiomerically enriched form.
(4) Selective reaction of a chiral reagent (including enzymes) with one enantiomer from a racemic mixture—termed a kinetic resolution.

The compounds of the present invention have utility as selective D3 agonists in the treatment of disease states. There are a number of compounds with activity as both D2 and D3 agonists; however the use of such compounds is associated with a large number of side effects including nausea, emesis, syncope, hypotension and bradycardia, some of which are a cause for serious concern.

It was previously held that the efficacy of the prior art compounds stemmed from their ability to agonise D2; however D2 agonism is implicated as a cause of the side effects detailed above.

The present invention provides a class of selective D3 agonists. Serendipitously, these have been found to be efficacious, whilst reducing the side effects associated with unselective prior art compounds.

Accordingly a further aspect of the invention provides a compound of formula (I) for use as a medicament.

Compounds of present invention are particularly useful in treating sexual dysfunction, female sexual dysfunction, including hypoactive sexual desire disorder, female sexual arousal disorder, female orgasmic disorder and sexual pain disorder; male erectile dysfunction, hypertension, neurodegeneration, depression, and psychiatric disorders.

Accordingly, the present invention provides for, the use of a compound of formula (I) in the preparation of a medicament for the treatment or prevention of sexual dysfunction.

The compounds of the present invention are useful in male sexual dysfunction, particularly male erectile dysfunction. Male erectile dysfunction (MED), otherwise known as male erectile disorder, is defined as:
“the inability to achieve and/or maintain a penile erection for satisfactory sexual performance” (NIH Consensus Development Panel on Impotence, 1993)”

It has been estimated that the prevalence of erectile dysfunction (ED) of all degrees (minimal, moderate and complete impotence) is 52% in men 40 to 70 years old, with higher rates in those older than 70 (Melman et al 1999, J. Urology, 161, p5-11). The condition has a significant negative impact on the quality of life of the individual and their partner, often resulting in increased anxiety and tension which leads to depression and low self-esteem. Whereas two decades ago, MED was primarily considered to be a psychological disorder (Benet et a/1994 Comp. Ther., 20: 669-673), it is now known that for the majority of individuals there is an underlying organic cause. As a result, much progress has been made in identifying the mechanism of normal penile erection and the pathophysiologies of MED.

Penile erection is a haemodynamic event which is dependent upon the balance of contraction and relaxation of the corpus cavernosal smooth muscle and vasculature of the penis (Lerner et al 1993, J. Urology, 149, 1256-1255). Corpus cavernosal smooth muscle is also referred to herein as corporal smooth muscle or in the plural sense corpus cavernosa. Relaxation of the corpus cavernosal smooth muscle leads to an increased blood flow into the trabecular spaces of the corpus cavemosa, causing them to expand against the surrounding tunica and compress the draining veins. This produces a vast elevation in blood pressure which results in an erection (Naylor, 1998, Br. J. Urology, 81, 424-431).

The changes that occur during the erectile process are complex and require a high degree of coordinated control involving the peripheral and central nervous systems, and the endocrine system (Naylor, 1998, Br. J. Urology, 81, 424-431). Corporal smooth muscle contraction is modulated by sympathetic noradrenergic innervation via activation of postsynaptic α₁ adrenoceptors. MED may be associated with an increase in the endogenous smooth muscle tone of the corpus cavernosum. However, the process of corporal smooth muscle relaxation is mediated partly by non-adrenergic, non-cholinergic (NANC) neurotransmission. There are a number of other NANC neurotransmitters found in the penis, other than NO, such as calcitonin gene related peptide (CGRP) and vasoactive intestinal peptide (VIP). The main relaxing factor responsible for mediating this relaxation is nitric oxide (NO), which is synthesised from L-arginine by nitric oxide synthase (NOS) (Taub et al 1993 Urology, 42, 698-704). It is thought that reducing corporal smooth muscle tone may aid NO to induce relaxation of the corpus cavernosum. During sexual arousal in the male, NO is released from neurones and the endothelium and binds to and activates soluble guanylate cyclase (sGC) located in the smooth muscle cells and endothelium, leading to an elevation in intracellular cyclic guanosine 3′,5′-monophosphate (cGMP) levels. This rise in CGMP leads to a relaxation of the corpus cavernosum due to a reduction in the intracellular calcium concentration ([Ca²⁺]ᵢ), via unknown mechanisms thought to involve protein kinase G activation (possibly due to activation of Ca²⁺ pumps and Ca²⁺-activated K⁺ channels).

Multiple potential sites have been identified within the central nervous system for the modulation of sexual behaviour. The key neurotransmitters are thought to be serotonin, norepinephrine, oxytocin, nitric oxide and dopamine. By mimicking the actions of one of these key neurotransmitters sexual function may be adjusted. Dopamine D3 receptors are expressed almost exclusively in the limbic area of the brain, regions involved in the reward, emotional and cognitive processes.

Without being bound by any theory, it appears that “due to its role in the control of locomotor activity, the integrity of the nigrostriatal dopaminergic pathway is also essential for the display of copulatory behaviour. Somehow, more specific to sexual function, it is likely that dopamine can trigger penile erection by acting on oxytocinergic neurons located in the paraventricular nucleus of the hypothalamus, and perhaps on the pro-erectile sacral parasympathetic nucleus within the spinal cord”. It now appears that the significant site is D3 and not as previously thought, D2.

In essence, D3 is an initiator of sexual behaviour.

Accordingly, the present invention provides for, the use of a compound of formula (I) in the preparation of a medicament for the treatment or prevention of erectile dysfunction.

Patients with mild to moderate MED should benefit from treatment with the compounds according to the present invention, and patients with severe MED may also respond. However, early investigations suggest that the responder rate of patients with mild, moderate and severe MED may be greater with a selective D3 agonist/PDE5 inhibitor combination. Mild, moderate and severe MED will be terms known to the man skilled in the art, but guidance can be found in The Journal of Urology, vol. 151, 54-61 (January 1994).

Early investigations suggest the below mentioned MED patient groups should benefit from treatment with a selective D3 agonist and a PDE5i (or other combination set out hereinafter). These patient groups, which are described in more detail in Clinical Andrology vol. 23, no.4, p773-782 and chapter 3 of the book by 1. Eardley and K. Sethia “Erectile Dysfunction-Current Investigation and Management, published by Mosby-Wolfe, are as follows: psychogenic, organic, vascular, endocrinologic, neurogenic, arteriogenic, drug-induced sexual dysfunction (lactogenic) and sexual dysfunction related to cavernosal factors, particularly venogenic causes.

Accordingly the present invention provides for the use of a compound of formula (I) in the preparation of a medicament in combination with a PDE5 inhibitor for the treatment of erectile dysfunction.

Suitable PDE5 inhibitors are described herein.

The compounds of the present invention are useful in the treatment or prevention of female sexual dysfunction (FSD), particularly female sexual arousal disorder (FSAD), hypoactive sexual desire disorder (HSDD; lack of interest in sex), FSAD with concomitant HSDD, and female orgasmic disorder (FOD; inability to achieve orgasm).

In accordance with the invention, FSD can be defined as the difficulty or inability of a woman to find satisfaction in sexual expression. FSD is a collective term for several diverse female sexual disorders (Leiblum, S. R. (1998)—Definition and classification of female sexual disorders. Int. J. Impotence Res., 10, S104-S106; Berman, J. R., Berman, L. & Goldstein, I. (1999)—Female sexual dysfunction: Incidence, pathophysiology, evaluations and treatment options. Urology, 54, 385-391.). The woman may have lack of desire, difficulty with arousal or orgasm, pain with intercourse or a combination of these problems. Several types of disease, medications, injuries or psychological problems can cause FSD. Treatments in development are targeted to treat specific subtypes of FSD, predominantly desire and arousal disorders.

The categories of FSD are best defined by contrasting them to the phases of normal female sexual response: desire, arousal and orgasm (Leiblum, S. R. (1998)—Definition and classification of female sexual disorders. Int. J. Impotence Res., 10, S104-S106). Desire or libido is the drive for sexual expression. Its manifestations often include sexual thoughts either when in the company of an interested partner or when exposed to other erotic stimuli. Arousal is the vascular response to sexual stimulation, an important component of which is genital engorgement and includes increased vaginal lubrication, elongation of the vagina and increased genital sensation/sensitivity. Orgasm is the release of sexual tension that has culminated during arousal.

Hence, FSD occurs when a woman has an inadequate or unsatisfactory response in any of these phases, usually desire, arousal or orgasm. FSD categories include hypoactive sexual desire disorder, sexual arousal disorder, orgasmic disorders and sexual pain disorders. Although the compounds of the invention will improve the genital response to sexual stimulation (as in female sexual arousal disorder), in doing so it may also improve the associated pain, distress and discomfort associated with intercourse and so treat other female sexual disorders.

Hypoactive sexual desire disorder is present if a woman has no or little desire to be sexual, and has no or few sexual thoughts or fantasies. This type of FSD can be caused by low testosterone levels, due either to natural menopause or to surgical menopause. Other causes include illness, medications, fatigue, depression and anxiety.

Female sexual arousal disorder (FSAD) is characterised by inadequate genital response to sexual stimulation. The genitalia do not undergo the engorgement that characterises normal sexual arousal. The vaginal walls are poorly lubricated, so that intercourse is painful. Orgasms may be impeded. Arousal disorder can be caused by reduced oestrogen at menopause or after childbirth and during lactation, as well as by illnesses, with vascular components such as diabetes and atherosclerosis. Other causes result from treatment with diuretics, antihistamines, antidepressants e.g. selective serotonin re-uptake inhibitors (SSRIs) or antihypertensive agents.

Sexual pain disorders (includes dyspareunia and vaginismus) is characterised by pain resulting from penetration and may be caused by medications which reduce lubrication, endometriosis, pelvic inflammatory disease, inflammatory bowel disease or urinary tract problems.

As previously discussed, D3 is thought to be an initiator of sexual behaviour. The clitoris is considered to be a homologue of the penis (Levin, R. J. (1991), Exp. Clin. Endocrinol., 98, 61-69); the same mechanism that provides provides an erectile response in the male produces an increase in genital blood flow in the female with an associated effect upon FSD. In addition there are changes in proceptivity and receptivity.

Thus, in accordance with a preferred aspect of the invention, there is provided use of a compound of formula (I) in the preparation of a medicament for the treatment or prophylaxis of female sexual dysfunction, more particularly hypoactive sexual desire disorder, female sexual arousal disorder, female orgasmic disorder and sexual pain disorder.

Preferably the compounds of formula (I) are useful in the treatment or prophylaxis of female sexual arousal disorder (FSAD), FSAD with concomitant hypoactive sexual desire disorder, orgasmic disorder, and hypoactive sexual desire disorder, and most preferably in the treatment or prophylaxis of female sexual arousal disorder.

In a preferred embodiment the compounds of formula (I) are useful in the treatment of a subject with female sexual arousal disorder and concomitant hypoactive sexual desire disorder.

The Diagnostic and Statistical Manual (DSM) IV of the American Psychiatric Association defines Female Sexual Arousal Disorder (FSAD) as being:
“ . . . a persistent or recurrent inability to attain or to maintain until completion of the sexual activity adequate lubrication-swelling response of sexual excitement. The disturbance must cause marked distress or interpersonal difficulty . . . . ”.

The arousal response consists of vasocongestion in the pelvis, vaginal lubrication and expansion and swelling of the external genitalia. The disturbance causes marked distress and/or interpersonal difficulty.

FSAD is a highly prevalent sexual disorder affecting pre-, per- and post-menopausal (±hormone replacement therapy (HRT)) women. It is associated with concomitant disorders such as depression, cardiovascular diseases, diabetes and urogenital (UG) disorders.

The primary consequences of FSAD are lack of engorgement/swelling, lack of lubrication and lack of pleasurable genital sensation. The secondary consequences of FSAD are reduced sexual desire, pain during intercourse and difficulty in achieving an orgasm.

It has recently been hypothesised that there is a vascular basis for at least a proportion of patients with symptoms of FSAD (Goldstein et al., Int. J. Impot. Res., 10, S84-S90, 1998) with animal data supporting this view (Park et al., Int. J. Impot. Res., 9, 27-37, 1997).

R. J. Levin teaches us that because “ . . . male and female genitalia develop embryologically from the common tissue anlagen, [that] male and female genital structures are argued to be homologues of one another. Thus the clitoris is the penile homologue and the labia homologues of the scrotal sac . . . . ” (Levin, R. J. (1991), Exp. Clin. Endocrinol., 98, 61-69).

Drug candidates for treating FSAD, which are under investigation for efficacy, are primarily erectile dysfunction therapies that promote circulation to male genitalia.

The compounds of the present invention are advantageous by providing a means for restoring a normal sexual arousal response—namely increased genital blood flow leading to vaginal, clitoral and labial engorgement. This will result in increased vaginal lubrication via plasma transudation, increased vaginal compliance and increased genital sensitivity. Hence, the present invention provides a means to restore, or potentiate, the normal sexual arousal response.

Thus, in accordance with a preferred aspect of the invention, there is provided use of a compound of formula (I) in the preparation of a medicament for the treatment or prophylaxis of female sexual arousal disorder and female sexual arousal disorder with concomitant hypoactive sexual desire disorder.

By female genitalia herein we mean: “The genital organs consist of an internal and external group. The internal organs are situated within the pelvis and consist of ovaries, the uterine tubes, uterus and the vagina. The external organs are superficial to the urogenital diaphragm and below the pelvic arch. They comprise the mons pubis, the labia majora and minora pudendi, the clitoris, the vestibule, the bulb of the vestibule, and the greater vestibular glands” (Gray's Anatomy, C. D. Clemente, 13^{th} American Edition).

The compounds of the invention find application in the following sub-populations of patients with FSD: the young, the elderly, pre-menopausal, peri-menopausal, post-menopausal women with or without hormone replacement therapy.

The compounds of the invention find application in patients with FSD arising from:—
i) Vasculogenic etiologies e.g. cardiovascular or atherosclerotic diseases, hypercholesterolemia, cigarette smoking, diabetes, hypertension, radiation and perineal trauma, traumatic injury to the iliohypogastric pudendal vascular system.
ii) Neurogenic etiologies such as spinal cord injuries or diseases of the central nervous system including multiple sclerosis, diabetes, Parkinsonism, cerebrovascular accidents, peripheral neuropathies, trauma or radical pelvic surgery.
iii) Hormonal/endocrine etiologies such as dysfunction of the hypothalamic/pituitary/gonadal axis, or dysfunction of the ovaries, dysfunction of the pancreas, surgical or medical castration, androgen deficiency, high circulating levels of prolactin e.g. hyperprolactinemia, natural menopause, premature ovarian failure, hyper and hypothyroidism.
iv) Psychogenic etiologies such as depression, obsessive compulsive disorder, anxiety disorder, postnatal depression/“Baby Blues”, emotional and relational issues, performance anxiety, marital discord, dysfunctional attitudes, sexual phobias, religious inhibition or traumatic past experiences.
v) Drug-induced sexual dysfunction resulting from therapy with selective serotonin reuptake inhibitors (SSR is) and other antidepressant therapies (tricyclics and major tranquillizers), anti-hypertensive therapies, sympatholytic drugs, chronic oral contraceptive pill therapy.

The Compounds of the present invention are also useful in the treatment of depression.

Dopamine D3 receptors are expressed almost exclusively in the limbic area of the brain, regions involved in reward, emotional and cognitive processes. Chronic treatment with several classes of antidepressants are known to increase the expression of D3 in the limbic area, and antidepressant effects of desipramine can be blocked by sulpride (D2/D3 antagonist) when injected to nucleus accumbens (area rich in D3) but not caudate-putamen (area rich in dopamine D2 receptors). In addition, antidepressant effects were observed preclinical models of depression and in patients treated with pramipexole, a D3-preferring D2/D3 agonist. The available information suggests that D3 receptors mediate the anti-depressant activity and that selective D3 receptor agonists represent a new class of antidepressant drugs. Since antidepressants are known to be effective in other psychiatric disorders, D3 agonists would have the potential to treat psychiatric diseases.

Suitable conditions include depression (e.g. depression in cancer patients, depression in Parkinson's patients, postmyocardial infarction depression, subsyndromal symptomatic depression, depression in infertile women, major depression, child abuse induced depression, post partum depression and grumpy old man syndrome), single episodic or recurrent major depressive disorders, dysthymic disorders, depressive neurosis and neurotic depression, melancholic depression including anorexia, weight loss, insomnia, early morning waking or psychomotor retardation; atypical depression (or reactive depression) including increased appetite, hypersomnia, psychomotor agitation or irritability, seasonal affective disorder and pediatric depression; bipolar disorders or manic depression, for example, bipolar I disorder, bipolar II disorder and cyclothymic disorder; conduct disorder; disruptive behavior disorder; trichotillomania, kleptomania, attention deficit hyperactivity disorder (ADHD); behavioral disturbances associated with mental retardation, autistic disorder; borderline personality disorder; avoidant personality disorder; anxiety disorders such as panic disorder with or without agoraphobia, agoraphobia without history of panic disorder, specific phobias, for example, specific animal phobias, social anxiety, social phobia, obsessive-compulsive disorder, stress disorders including post-traumatic stress disorder and acute stress disorder, and generalized anxiety disorders; emotional lability, pathological crying; schizophrenia and other psychotic disorders, for example, schizophreniform disorders, schizoaffective disorders, delusional disorders, brief psychotic disorders, shared psychotic disorders, psychotic disorders with delusions or hallucinations, psychotic episodes of anxiety, anxiety associated with psychosis, psychotic mood disorders such as severe major depressive disorder; mood disorders associated with psychotic disorders such as acute mania and depression associated with bipolar disorder; mood disorders associated with schizophrenia; eating disorders (e.g. anorexia nervosa and bulimia nervosa), obesity; movement disorders such as akinesias, dyskinesias, including familial paroxysmal dyskinesias, spasticities, Tourette's syndrome, Scott syndrome, PALSYS and akinetic-rigid syndrome; extra-pyramidal movement disorders such as medication-induced movement disorders, for example, neuroleptic-induced Parkinsonism, neuroleptic malignant syndrome, neuroleptic-induced acute dystonia, neuroleptic-induced acute akathisia, neuroleptic-induced tardive dyskinesia and medication-induced postural tremour; chemical dependencies and addictions (e.g., dependencies on, or addictions to, alcohol, heroin, cocaine, benzodiazepines, nicotine, or phenobarbitol) and behavioral addictions such as an addiction to gambling; ocular disorders such as glaucoma and ischemic retinopathy; sleeping disorder (cataplexy) and shock.

In a further preferred embodiment, the present invention provides for the use of a compound of formula (I) in the preparation of a medicament for the treatment of depression or psychiatric disorders.

Suitable depressive conditions and psychiatric disorders are described above.

In an additional further embodiment, the invention provides for the use of compounds of formula I in the preparation of a medicament for the treatment of obesity.

The compounds of the present invention also have utility in the treatment of neurodegeneration; sources of neurodegeneration include neurotoxin poisoning; vision loss caused by neurodegeneration of the visual pathway, such as by a stroke in the visual pathway eg in retina, optic nerve and/or occipital lobe; epileptic seizures; and from impairment of glucose and/or oxygen supply to the brain.

Conditions related to neurodegeneration include Restless Leg Syndrome, Huntington's disease, Multiple Sclerosis, mild cognitive impairment, Down's syndrome, stroke, Hereditary Cerebral Hemorrhage with Amyloidosis of the Dutch-Type, cerebral amyloid angiopathy, delirium, dementia, age-related cognitive decline (ARCD), and amnestic and other cognitive or neurodegenerative disorders, such as Parkinson's disease (PD), Huntington's disease (HD), Alzheimer's disease, senile dementia, dementia of the Alzheimer's type, memory disorders, loss of executive function, vascular dementia, dementias of mixed vascular and degenerative origin, dementia associated with Parkinson's disease, dementia associated with progressive supranuclear palsy, dementia associated with cortical basal degeneration, multi-infarct dementia, alcoholic dementia or other drug-related dementia, dementia associated with intracranial tumors or cerebral trauma, dementia associated with Huntington's disease, Pick's disease, Creutzfeldt-Jakob disease, HIV or AIDS-related dementia, diffuse Lewy body type of Alzheimer's disease, frontotemporal dementias with parkinsonism (FTDP), head trauma, spinal cord injury, demyelinating diseases of the nervous system, peripheral neuropathy, pain, cerebral amyloid angiopathy, amyotrophic lateral sclerosis, multiple sclerosis, dyskinesia associated with dopamine agonist therapy, mental retardation, learning disorders, including reading disorder, mathematics disorder, or a disorder of written expression; age-related cognitive decline, amnesic disorders, neuroleptic-induced parkinsonism, tardive dyskinesias, and acute and chronic neurodegenerative disorders.

Accordingly the present invention provides for the use of a compound of formula (I) in the preparation of a medicament for the treatment of neurodegeneration.

Suitable neurodegenerative conditions are described above.

In addition to their role in treating Sexual dysfunction, depression, neurodegeneration and psychiatric disorders, the compounds of the present invention are likely to be efficacious in a number of additional indications.

Accordingly, the present invention provides for the use of compounds of formula (I), in the preparation of a medicament for the treatment of hypertension, premature ejaculation, obesity, cluster headache, migraine, pain, endocrine disorders (e.g. hyperprolactinaemia), vasospasm (particularly in the cerebral vasculature), cerebellar ataxia, gastrointestinal tract disorders (involving changes in motility and secretion), premenstrual syndrome, fibromyalgia syndrome, stress incontinence, trichotillomania and chronic paroxysmal hemicrania, headache (associated with vascular disorders).

It is to be appreciated that all references herein to treatment include curative, palliative and prophylactic treatment.

D3/D2 Agonist Assay

Activity at the dopamine D3 receptor may be determined using the methods described in WO 2004/052372. Using this assay, the compounds of the present invention all exhibit a functional potency at D3 receptor expressed as an EC50, lower than 1000 nM and a 10 fold selectivity for D3 over D2. Selectivity is calculated as the D2 EC50 value divided by the D3 EC50 value. Where the value of the D2 EC50 was >10000, a figure of 10000 was used in the calculation.

The compound of example 14 has a functional potency at the D3 receptor, expressed as an EC50, of 20 nM, with an Emax (maximal response value) of 98% (relative to the maximal effect of standard agent pramipexole). Against the D2 receptor this compound gave only a 22% response (relative to the maximal effect of pramipexole) at 1000 nM.

Suitable auxiliary active agents for use in the combinations of the present invention include:
1) Naturally occurring or synthetic prostaglandins or esters thereof. Suitable prostaglandins for use herein include compounds such as alprostadil, prostaglandin E₁,prostaglandin E₀, 13, 14-dihydroprosta glandin E₁, prostaglandin E₂, eprostinol, natural synthetic and semi-synthetic prostaglandins and derivatives thereof including those described in WO-00033825 and/or U.S. Pat. No. 6,037,346 issued on 14 Mar. 2000 all incorporated herein by reference, PGE₀, PGE₁, PGA₁, PGB₁, PGF₁ α, 19-hydroxy PGA₁, 19-hydroxy-PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, sulprostune, tiaprost and moxisylate;
2) α—adrenergic receptor antagonist compounds also known as α—adrenoceptors or α-receptors or α-blockers. Suitable compounds for use herein include: the α-adrenergic receptor blockers as described in PCT application WO99/30697 published on 14 Jun. 1998, the disclosures of which relating to α-adrenergic receptors are incorporated herein by reference and include, selective α₁-adrenoceptor or α₂-adrenoceptor blockers and non-selective adrenoceptor blockers, suitable α₁-adrenoceptor blockers include: phentolamine, phentolamine mesylate, trazodone, alfuzosin, indoramin, naftopidil, tamsulosin, dapiprazole, phenoxybenzamine, idazoxan, efaraxan, yohimbine, rauwolfa alkaloids, Recordati 15/2739, SNAP 1069, SNAP 5089, RS17053, SL 89.0591, doxazosin, terazosin, abanoquil and prazosin; α₂-blocker blockers from U.S. Pat. No. 6,037,346 [14 Mar. 2000] dibenamine, tolazoline, trimazosin and dibenamine; α-adrenergic receptors as described in U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference; α₂-Adrenoceptor blockers include: clonidine, papaverine, papaverine hydrochloride, optionally in the presence of a cariotonic agent such as pirxamine;
3) NO-donor (NO-agonist) compounds. Suitable NO-donor compounds for use herein include organic nitrates, such as mono- di or tri-nitrates or organic nitrate esters including glyceryl trinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy-L-arginine, amyInitrate, linsidomine, linsidomine chlorohydrate, (SIN-1) S-nitroso-N-cysteine, diazenium diolates,(NONOates), 1,5-pentanedinitrate, L-arginene, ginseng, zizphi fructus, molsidomine, Re-2047, nitrosylated maxisylyte derivatives such as NMI-678-11 and NMI-937 as described in published PCT application WO 0012075;
4) Potassium channel openers or modulators. Suitable potassium channel openers/modulators for use herein include nicorandil, cromokalim, levcromakalim, lemakalim, pinacidil, cliazoxide, minoxidil, charybdotoxin, glyburide, 4-amini pyridine, BaCl₂;
5) Vasodilator agents. Suitable vasodilator agents for use herein include nimodepine, pinacidil, cyclandelate, isoxsuprine, chloroprumazine, Rec 15/2739, trazodone;
6) Thromboxane A2 agonists;
7) CNS active agents;
8) Ergot alkoloids; Suitable ergot alkaloids are described in U.S. Pat. No. 6,037,346 issued on 14 Mar. 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride and terguride;
9) Compounds which modulate the action of natruretic factors in particular atrial naturetic factor (also known as atrial naturetic peptide), B type and C type naturetic factors such as inhibitors or neutral endopeptidase;
10) Compounds which inhibit angiotensin-converting enzyme such as enapril, and combined inhibitors of angiotensin-converting enzyme and neutral endopeptidase such as omapatrilat.
11) Angiotensin receptor antagonists such as losartan;
12) Substrates for NO-synthase, such as L-arginine;
13) Calcium channel blockers such as amlodipine;
14) Antagonists of endothelin receptors and inhibitors or endothelin-converting enzyme;
15) Cholesterol lowering agents such as statins (e.g. atorvastatin/Lipitor-trade mark) and fibrates;
16) Antiplatelet and antithrombotic agents, e.g. tPA, uPA, warfarin, hirudin and other thrombin inhibitors, heparin, thromboplastin activating factor inhibitors;
17) Insulin sensitising agents such as rezulin and hypoglycaemic agents such as glipizide;
18) Acetylcholinesterase inhibitors such as donezipil;
19) Steroidal or non-steroidal anti-inflammatory agents;
20) Estrogen receptor modulators and/or estrogen agonists and/or estrogen antagonists, preferably raloxifene or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656;
21) A PDE inhibitor, more particularly a PDE 2, 3, 4, 5, 7 or 8 inhibitor, preferably PDE2 or PDE5 inhibitor and most preferably a PDE5 inhibitor (see hereinafter), said inhibitors preferably having an IC50 against the respective enzyme of less than 100 nM (with the proviso that PDE 3 and 4 inhibitors are only administered topically or by injection to the penis);
22) Vasoactive intestinal protein (VIP), VIP mimetic, VIP analogue, more particularly mediated by one or more of the VIP receptor subtypes VPAC1, VPAC or PACAP (pituitory adenylate cyclase activating peptide), one or more of a VIP receptor agonist or a VIP analogue (e.g. Ro-125-1553) or a VIP fragment, one or more of a α-adrenoceptor antagonist with VIP combination (e.g. Invicorp, Aviptadil);
23) A melanocortin receptor (particularly of the MC3 or MC4 subtype) agonist or modulator or melanocortin enhance, such as melanotan 11, PT-14, PT-141 or compounds claimed in WO-09964002, WO-00074679, WO-09955679, WO-00105401, WO-00058361, WO-00114879, WO-00113112, WO-09954358;
24) A serotonin receptor agonist, antagonist or modulator, more particularly agonists, antagonists or modulators for 5HT1A (including VML 670), 5HT2A, 5HT2C, 5HT3 and/or 5HT6 receptors, including those described in WO-09902159, WO-00002550 and/or WO-00028993;
25) A testosterone replacement agent (including dehydroandrostendione), testosternone (Tostrelle), dihydrotestosterone or a testosterone implant;
26) Estrogen, estrogen and medroxyprogesterone or medroxyprogesterone acetate (MPA) (i.e. as a combination), or estrogen and methyl testosterone hormone replacement therapy agent (e.g. HRT especially Premarin, Cenestin, Oestrofeminal, Equin, Estrace, Estrofem, Elleste Solo, Estring, Eastraderm TTS, Eastraderm Matrix, Dermestril, Premphase, Preempro, Prempak, Premique, Estratest, Estratest HS, Tibolone);
27) A modulator of transporters for noradrenaline, dopamine and/or serotonin, such as bupropion, GW-320659;
28) A purinergic receptor agonist and/or modulator;
29) A neurokinin (NK) receptor antagonist, including those described in WO-09964008;
30) An opioid receptor agonist, antagonist or modulator, preferably agonists for the ORL-1 receptor;
31) An agonist, antagonist or modulator for oxytocin receptors, preferably a selective oxytocin agonist or modulator;
32) Modulators of cannabinoid receptors;
33) A SEP inhibitor (SEPi), for instance a SEPi having an IC₅₀ at less than 100 nanomolar, more preferably, at less than 50 nanomolar.
   Preferably, the SEP inhibitors according to the present invention have greater than 30-fold, more preferably greater than 50-fold selectivity for SEP over neutral endopeptidase NEP EC 3.4.24.11 and angiotensin converting enzyme (ACE). Preferably the SEPi also has a greater than 100-fold selectivity over endothelin converting enzyme (ECE).
34) An antagonist or modulator for the NPY (particularly Y1 and Y5 subtype) receptor.
35) A Sex Hormone Binding Globulin antagonist or modulator that inhibits estrogens and/or androgens from being bound.
6) An arginase I inhibitor,
37) An agonist, antagonist or modulator for vassopressin receptors, preferably selective for the V1a receptor
38) A PDE5 Inhibitor. Suitable PDE5 inhibitors include:
   5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil), particularly sildenafil citrate;
   (6R, 12aR)-2,3,6,7,12,12α-hexahydro-2-methyl-6-(3,4-methylenedioxyphenyl)-pyrazino[2′, 1′:6,1]pyrido[3,4-b]indole-1,4-dione (IC-351 or tadalafil);
   2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil); 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one; 5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one; and 5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one; 4-[(3-chloro-4-methoxybenzyl)amino]-2-[(2S)-2-(hydroxymethyl)pyrrolidin-1-yl]-N-(pyrimidin-2-ylmethyl)pyrimidine-5-carboxamide (TA-1790); 3-(1-methyl-7-oxo-3-propyl-6,7-dihydro-1H-pyrazolo[4,3-d]pyrimidin-5-yl)-N-[2-(1-methylpyrrolidin-2-yl)ethyl]-4-propoxybenzenesulfonamide (DA 8159) and pharmaceutically acceptable salts thereof.
39) A selective dopamine D4 receptor agonist such as 2-[(4-pyridin-2-ylpiperazin-1-yl)methyl]-1H-benzimidazole (ABT724).

By cross reference herein to compounds contained in patents and patent applications which can be used in accordance with invention, we mean the therapeutically active compounds as defined in the claims (in particular of claim 1) and the specific examples (all of which is incorporated herein by reference).

If a combination of active agents is administered, then they may be administered simultaneously, separately or sequentially.

The compounds of formula I should be assessed for their biopharmaceutical properties, such as solubility and solution stability (across pH), permeability, etc., in order to select the most appropriate dosage form and route of administration for treatment of the proposed indication.

Compounds of the invention intended for pharmaceutical use may be administered as crystalline or amorphous products. They may be obtained, for example, as solid plugs, powders, or films by methods such as precipitation, crystallization, freeze drying, spray drying, or evaporative drying. Microwave or radio frequency drying may be used for this purpose.

They may be administered alone or in combination with one or more other compounds of the invention or in combination with one or more other drugs (or as any combination thereof). Generally, they will be administered as a formulation in association with one or more pharmaceutically acceptable excipients. The term ‘excipient’ is used herein to describe any ingredient other than the compound(s) of the invention. The choice of excipient will to a large extent depend on factors such as the particular mode of administration, the effect of the excipient on solubility and stability, and the nature of the dosage form.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Accordingly the present invention provides for a pharmaceutical composition comprising a compound of formula (I), and a pharmaceutically acceptable diluent or carrier.

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula 1, a film-forming polymer, a binder, a solvent, a humectant, a plasticiser, a stabiliser or emulsifier, a viscosity-modifying agent and a solvent. Some components of the formulation may perform more than one function.

The compound of formula I may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula I may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

The compounds of the invention may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular, intrasynovial and subcutaneous. Suitable devices for parenteral administration include needle (including microneedle) injectors, needle-free injectors and infusion techniques.

Parenteral formulations are typically aqueous solutions which may contain excipients such as salts, carbohydrates and buffering agents (preferably to a pH of from 3 to 9), but, for some applications, they may be more suitably formulated as a sterile non-aqueous solution or as a dried form to be used in conjunction with a suitable vehicle such as sterile, pyrogen-free water.

The preparation of parenteral formulations under sterile conditions, for example, by lyophilisation, may readily be accomplished using standard pharmaceutical techniques well known to those skilled in the art.

The solubility of compounds of formula I used in the preparation of parenteral solutions may be increased by the use of appropriate formulation techniques, such as the incorporation of solubility-enhancing agents.

Formulations for parenteral administration may be formulated to be immediate and/or modified release. Modified release formulations include delayed-, sustained-, pulsed-, controlled-, targeted and programmed release. Thus compounds of the invention may be formulated as a suspension or as a solid, semi-solid, or thixotropic liquid for administration as an implanted depot providing modified release of the active compound. Examples of such formulations include drug-coated stents and semi-solids and suspensions comprising drug-loaded poly(dl-lactic-coglycolic)acid (PGLA) microspheres.

The compounds of the invention may also be administered topically, (intra)dermally, or transdermally to the skin or mucosa. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated—see, for example, J Pharm Sci, 88 (10), 955-958, by Finnin and Morgan (October 1999).

Other means of topical administration include delivery by electroporation, iontophoresis, phonophoresis, sonophoresis and microneedle or needle-free (e.g. Powderject™, Bioject™, etc.) injection.

Formulations for topical administration may be formulated to be immediate and/or modified release. Modified release formulations include delayed-, sustained-, pulsed-, controlled-, targeted and programmed release.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound(s) of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula I, propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for inhaled/intranasal administration.

Formulations for inhaled/intranasal administration may be formulated to be immediate and/or modified release using, for example, PGLA. Modified release formulations include delayed-, sustained-, pulsed-, controlled-, targeted and programmed release.

In the case of dry powder inhalers and aerosols, the dosage unit is determined by means of a valve which delivers a metered amount. Units in accordance with the invention are typically arranged to administer a metered dose or “puff” containing from . . . to . . . μg of the compound of formula I. The overall daily dose will typically be in the range . . . μg to . . . mg which may be administered in a single dose or, more usually, as divided doses throughout the day.

The compounds of the invention may be administered rectally or vaginally, for example, in the form of a suppository, pessary, or enema. Cocoa butter is a traditional suppository base, but various alternatives may be used as appropriate.

Formulations for rectal/vaginal administration may be formulated to be immediate and/or modified release. Modified release formulations include delayed-, sustained-, pulsed-, controlled-, targeted and programmed release.

The compounds of the invention may also be administered directly to the eye or ear, typically in the form of drops of a micronised suspension or solution in isotonic, pH-adjusted, sterile saline. Other formulations suitable for ocular and aural administration include ointments, gels, biodegradable (e.g. absorbable gel sponges, collagen) and non-biodegradable (e.g. silicone) implants, wafers, lenses and particulate or vesicular systems, such as niosomes or liposomes. A polymer such as crossed-linked polyacrylic acid, polyvinylalcohol, hyaluronic acid, a cellulosic polymer, for example, hydroxypropylmethylcellulose, hydroxyethylcellulose, or methyl cellulose, or a heteropolysaccharide polymer, for example, gelan gum, may be incorporated together with a preservative, such as benzalkonium chloride. Such formulations may also be delivered by iontophoresis. Formulations for ocular/aural administration may be formulated to be immediate and/or modified release. Modified release formulations include delayed-, sustained-, pulsed-, controlled-, targeted, or programmed release.

The compounds of the invention may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration.

Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in International Patent Applications Nos. WO 91/11172, WO 94/02518 and WO 98/55148.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula I in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

The invention is illustrated by the following non-limiting examples in which the following abbreviations and definitions are used:
αD optical rotation at 587 nm.
Ac₂O acetic anhydride
APCI atmospheric pressure chemical ionisation
Arbacel® filter agent
br broad
Boc tert-butoxycarbonyl
Bu butyl
CDCl₃ chloroform-d1
CD₃OD methanol-d4
δ chemical shift
d doublet
dd double doublet
DCM dichloromethane
DMF N,N-dimethylformamide
DMSO dimethylsulfoxide
eq (molar) equivalents
ESI electrospray ionisation
Et ethyl
EtOAc ethyl acetate
h hours
HCl hydrogen chloride
HPLC high performance liquid chromatography
HR M/S high resolution mass spectrum
IPA isopropylalcohol
KOAc potassium acetate
m multiplet
Me methyl
MeCN acetonitrile
M/S mass spectrum
min minutes
NMR nuclear magnetic resonance
q quartet
r.t. room temperature
s singlet
sat saturated
t triplet
td triplet of doublets
Tf trifluoromethanesulfonyl
TFA trifluoroacetic acid
THF tetrahydrofuran
TIPS triisopropylsilyl
TLC/t.l.c thin layer chromatography

¹H Nuclear magnetic resonance (NMR) spectra were in all cases consistent with the proposed structures. Characteristic chemical shifts (δ) are given in parts-per-million □_{H} downfield from tetramethylsilane using conventional abbreviations for designation of major peaks: e.g. s, singlet; d, doublet; t, triplet; q, quartet; m, multiplet; br, broad. The following abbreviations have been used for common solvents: CDCl₃, deuterochloroform; DMSO, dimethylsulfoxide. The abbreviation psi means pounds per square inch and LRMS means low resolution mass spectrometry. Where thin layer chromatography (TLC) has been used it refers to silica gel TLC using silica gel 60 F₂₅₄ plates, R_{f} is the distance travelled by a compound divided by the distance travelled by the solvent front on a TLC plate.

### EXAMPLE 1

### 5-[(2R)-4-Benzylmorpholin-2-yl]pyridin-2-amine

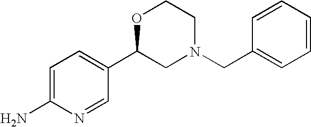

The morpholine from preparation 7 (2.05 g, 6 mmol) was dissolved in ethanol (75 mL), hydroxylamine hydrochloride (2.05 g, 30 mmol) was added and the mixture heated at 80° C. overnight (−16 h). After cooling to room temperature, the reaction mixture was evaporated to dryness to a yellow oily residue which was purified by flash chromatography on silica gel eluting with dichloromethane/methanol/0.880 NH₃ 98:2:0 increasing polarity to 95:5:0 then 95:5:0.5, then 90:10:1 to afford the title compound (645 mg, 40%)

¹H NMR (400 MHz, CDCl₃) δ_{H} 8.01 (1H, s), 7.43 (1H, d), 7.33 (5H, m), 6.46 (1H, d), 4.45 (3H brm), 3.96 (1H, d), 3.8 (1H, t), 3.54 (2H, s), 2.84 (1H, d), 2.74 (1H, d), 2.26 (1H, m), 2.12 (1H, t)

MS (APCI⁺) 270 (MH⁺)

### EXAMPLE 2

### 5-[(2R)-Morpholin-2-yl]pyridin-2-amine

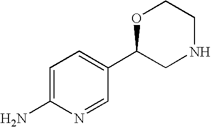

The benzyl morpholine from example 1 (990 mg, 3.7 mmol) was dissolved in methanol (20 mL) ammonium formate (1.16 g, 18.5 mmol) followed by 10% Pd on carbon (495 mg) were added and the mixture heated at reflux for 2 h. The cooled reaction mixture was filtered through a plug of arbocel® and evaporated to provide an orange solid (1.49 g). This material was purified by flash chromatography on silica gel (compound pre-absorbed onto silica) eluting with dichloromethane/methanol/0.880 NH₃ 90:10:1, to afford the title compound as a white solid (467 mg, 70%).

¹H NMR (400 MHz, CD₃OD) δ_{H} 7.82 (1H, s), 7.45 (1H, d), 6.58 (1H, d), 4.34 (1H, d), 3.95 (1H, d), 3.72 (1H, t), 2.95-2.80 (3H, m) 2.7 (1H, t)

MS (APCI⁺) 180 (MH⁺)

[α]_{D}²⁵ −39.4 (c=0.12, MeOH)

### EXAMPLE 3

### 5-[(2R)-4(3-Phenylpropyl)morpholin-2-yl]pyridin-2-amine

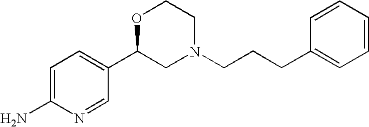

The morpholine from example 2 (80 mg, 0.45 mmol) was dissolved in tetrahydrofuran (15 mL) and 3-phenylpropionaldehyde (59 μL, 0.45 mmol) was added as a solution in tetrahydrofuran (15 mL) over 15 minutes. After the addition was complete, sodium triacetoxyborohydride (227 mg, 1 mmol) was added and the reaction mixture stirred at room temperature for 6 h. The reaction mixture was then diluted with saturated sodium hydrogencarbonate solution (50 mL) and extracted with ethyl acetate (2×50 mL). The combined organic fractions were dried (MgSO₄), filtered and evaporated to provide a yellow oil. Purification by flash chromatography on silica gel eluting with dichloromethane/methanol/0.880 NH₃ 98:2:0.2 increasing polarity to 95:5:0.5 afforded the title compound (51 mg, 38%)

¹H NMR (400 MHz, CD₃OD) δ_{H} 7.86 (1H, d), 7.45 (1H, dd), 7.27-7.10 (5H, m), 6.55 (1H, d), 4.39 (1H, d), 3.95 (1H, d), 3.75 (1H, t), 2.83 (2H, m), 2.64 (2H, t), 2.41 (2H, t), 2.20 (1H, m), 2.05 (1H, t), 1.84 (2H, m)

MS (APCI⁺) 298 (MH⁺)

[α]_{D}²⁵ +6.9 (c=0.13, MeOH)

### EXAMPLE 4

### 5-[(2R)-4-butylmorpholin-2-yl]pyridin-2-amine

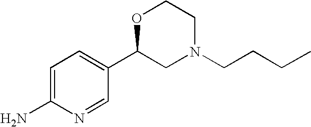

The morpholine from example 2 (80 mg, 0.45 mmol) was mixed with tetrahydrofuran (10 mL) (only partly soluble) and butyraldehyde (40 μL, 0.45 mmol) was added, resulting in a homogeneous solution. The reaction mixture was stirred for further 30 minutes before the addition of sodium triacetoxyborohydride (227 mg, 1 mmol). The reaction mixture was then stirred at room temperature overnight (˜16 h) before diluted with saturated sodium hydrogencarbonate solution (100 mL) and extracted with ethyl acetate (100 mL). The combined organic layer was separated, dried (MgSO₄), filtered and evaporated to provide a yellow oil. Purification by flash chromatography on silica gel eluting with dichloromethane/methanol/0.880 NH₃ 98:2:0.2 increasing polarity to 95:5:0.5 afforded the title compound (17 mg, 16%)

¹H NMR (400 MHz, CD₃OD) δ_{H} 7.86 (1H, s), 7.47 (1H, d), 6.55 (1H, d), 4.39 (1H, d), 3.98 (1H, d), 3.76 (1H, t), 2.86 (2H, t), 2.41 (2H, t), 2.21 (1H, t), 2.07 (1H, t), 1.50 (2H, m), 1.35 (2H, m), 0.95 (3H, t)

MS (APCI⁺) 236 (MH⁺)

### EXAMPLE 5

### 5-[(2R)-4-pentylmorpholin-2-yl]pyridin-2-amine

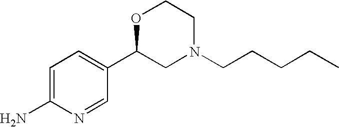

The morpholine from example 2 (80 mg, 0.45 mmol) was mixed with tetrahydrofuran (15 mL) and pentanal (47 μL, 0.45 mmol) was added dropwise as a solution in tetrahydrofuran (15 mL) over 15 minutes. After the addition was complete, sodium triacetoxyborohydride (227 mg, 1 mmol) was added and the reaction mixture stirred at room temperature overnight (˜16 h). The reaction mixture was then diluted with saturated sodium hydrogencarbonate solution (75 mL) and extracted with ethyl acetate (100 mL). The combined organic layer was separated, dried (MgSO₄), filtered and evaporated to provide a yellow oil. Purification by flash chromatography on silica gel eluting with dichloromethane/methanol/0.880 NH₃ 98:2:0.2 increasing polarity to 95:5:0.5 afforded the title compound (67 mg, 61%)

¹H NMR (400 MHz, CD₃OD) δ_{H} 7.86 (1H, s), 7.46 (1H, d), 6.55 (1H, d), 4.41 (1H, d), 3.94 (1H, d), 3.77 (1H, t), 2.86 (2H, t), 2.39 (2H, t), 2.21 (1H, t), 2.06 (1H, t), 1.54 (2H, m), 1.34 (4H, m), 0.92 (3H, t)

MS (APCI⁺) 250 (MH⁺)

[α]_{D}²⁵ +4.42 (c=0.13, MeOH)

### EXAMPLE 6

### 5-[(2R)-4-(2-phenylethyl)morpholin-2-yl]pyridin-2-amine

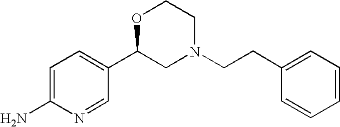

The morpholine from example 2 (80 mg, 0.45 mmol) was mixed with tetrahydrofuran (15 mL) and phenylacetaldehyde (52 μL, 0.45 mmol) was added dropwise as a solution in tetrahydrofuran (15 mL) over 15 minutes. After the addition was complete, the reaction mixture was allowed to stir at room temperature for 1 h before the addition of sodium triacetoxyborohydride (227 mg, 1 mmol). The reaction mixture was stirred at room temperature overnight (˜16 h) and then diluted with saturated sodium hydrogencarbonate solution (100 mL) and extracted with ethyl acetate (100 mL). The combined organic layer was separated, dried (MgSO₄), filtered and evaporated to provide a yellow oil. Purification by flash chromatography on silica gel eluting with dichloromethane/methanol/0.880 NH₃ 98:2:0.2 afforded the title compound (31 mg, 24%)

¹H NMR (400 MHz, CD₃OD) δ_{H} 7.87 (1H, s), 7.47 (1H, d), 7.20 (5H, m) 6.55 (1H, d), 4.42 (1H, d), 3.97 (1H, d), 3.78 (1H, t), 2.93 (2H, t) 2.82 (2H, m), 2.66 (2H, t) 2.30 (2H, t), 2.21 (1H, t), 2.15 (1H, t)

MS (APCI⁺) 284 (MH⁺)

### EXAMPLE 7a

### 5-[(2R,5S)-5-Methylmorpholin-2-yl]pyridin-2-amine

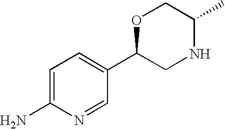

The material from preparation 10 (410 mg, 1.25 mmol) was dissolved in ethanol (10 mL), 5% Pd on carbon (40 mg) was added and the mixture hydrogenated at room temperature overnight at 1 atmosphere. The mixture was then filtered through a plug of arbocel®, washing the plug with ethanol and the combined filtrates and washings were evaporated to a pale yellow solid. Purification by flash chromatography on silica gel eluting with dichloromethane/methanol/0.880 NH₃ 93:7:0.5 afforded the title compound as a white solid (110 mg, 45%)

¹H NMR: δ_{H} (400 MHz, CD₃OD) 7.85 (1H, d), 7.45 (1H, dd), 6.55 (1H, d), 4.29 (1H, m), 3.90 (1H, m), 3.30 (1H, m), 2.95-2.85 (2H, m), 2.75 (1H, m), 1.01 (3H, d)

MS (ES⁺) 194 (MH⁺)

Alternatively a the morpholine ring may be formed by the following conditions to provide a mixture of diastereoisomers:

### EXAMPLE 7b

### 5-[(5S)-5-Methylmorpholin-2-yl]pyridin-2-amine (diastereomer mixture)

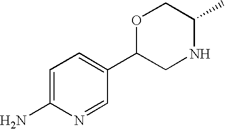

Diol from preparation 11 (1.26 g, 5.96 mmol) was dissolved in dichloromethane (20 mL) and treated with concentrated sulfuric acid (8 mL) at room temperature. The mixture was stirred for 2 h before being quenched by cautious addition of water, basification with 880 NH₃ to pH ·9 and extracted with dichloromethane (2×150 mL). The combined organics were dried over magnesium sulfate, filtered and evaporated to provide the title compounds as a 3:1 mixture of (R, S) and (S, S) diastereomers respectively.

¹H NMR: δ_{H} (400 MHz, CD₃OD) 7.85 (1H, m), 7.52-7.45 (1H, 2×dd), 6.60-6.52 (1H, 2×d), 4.38-4.22 (1H, 2×dd), 3.95-3.80 (1H, 2×dd), 3.30 (1H, m), 3.10-2.83 (2H, m), 2.75 (1H, m), 1.39-0.99 (3H, 2×d)

### EXAMPLES 8 AND 9

A mixture of the morpholine compounds from Example 7b (240 mg, 1.2 mmol) was dissolved in tetrahydrofuran (45 mL) and to the stirred solution was added 3-phenylpropionaldehyde (165 μL, 1.2 mmol) dropwise as a solution in tetrahydrofuran (45 mL). Once the addition was complete, sodium triacetoxyborohydride (270 mg, 1.2 mmol) was added and the reaction mixture left to stir at room temperature overnight. The solvent was evaporated and the diluted with water (30 mL) and extracted with dichloromethane (2×100 mL). The combined organic fractions were dried (MgSO₄), filtered and evaporated to provide a clear oil of a ca. 2:1 mixture of trans:cis diastereoisomers.

The diastereoisomers were separated by HPLC on a Chiralcel AD-H column with a mobile phase of methanol:ethanol 50:50 and a flow rate of 15 ml/min

### EXAMPLE 8

### Diastereomer 1

### 5-[(2R,5S)-5-Methyl-4-(3-phenylpropyl)morpholin-2-yl]pyridin-2-amine

Retention time 4.80 min

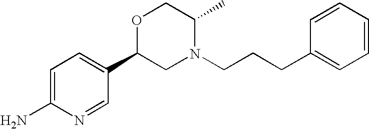

¹H NMR: δ_{H} (400 MHz, CD₃OD) 7.83 (1H, s), 7.44 (1H, d), 7.29-7.08 (m, 5H) 6.52 (1H, d), 4.40 (1H, d), 3.79 (1H, d), 3.30 (1H, m), 2.91-2.78 (2H, m), 2.60-2.50 (2H, m), 2.40 (1H, m), 2.29 (1H, m), 2.19 (1H, m), 1.88-1.68 (2H, m), 0.95 (3H, d)

MS (APCI⁺) 312 (MH⁺)

### EXAMPLE 9 (Diastereomer 2)

### 5-[(2S,5S)-5-Methyl-4-(3-phenylpropyl)morpholin-2-yl]pyridin-2-amine

Retention time 7.60 min

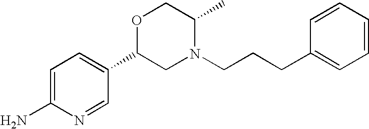

¹H NMR: δ_{H} (400 MHz, CD₃OD) 7.89 (1H, s), 7.49 (1H, d), 7.29-7.09 (5H, m), 6.55 (1H, d), 4.42 (1H, m), 3.87 (1H, m), 3.72 (1H, d), 2.90 (1H, m), 2.70-2.62 (2H, m), 2.60-2.42 (4H, m), 1.90-1.75 (2H, m), 1.09 (3H, d)

MS (APCI⁺) 312 (MH⁺)

### EXAMPLES 10 AND 11

The diol from preparation 31 (350 mg, 1.3 mmol) was dissolved in dichloromethane (5 mL) and to the stirred solution was added concentrated H₂SO₄ (2.5 mL). The reaction mixture left to stir at room temperature for 2 h then quenched by the cautious addition of water (10 mL) then basified by the addition of 0.880 NH₃ to pH 8-9. The mixture was then extracted with dichloromethane (2×50 mL) and the combined extracts were dried (MgSO₄), filtered and evaporated to provide a brown oil of a mixture of cis and trans morpholine diastereomers (275 mg, 85%)

MS (ES⁺) 250 (MH⁺)

The sample of mixed diastereoisomers was subjected HPLC using a Chiralcel OD-H column, mobile phase was 30:70 IPA/Hexane with diethylamine 0.1%, at a flow rate of 20 mL/min.

### EXAMPLE 10 (Diastereomer 1)

### 5-[(2S,5S)-4-Butyl-5-methylmorpholin-2-yl]pyridin-2-amine

Retention time 4.90 min

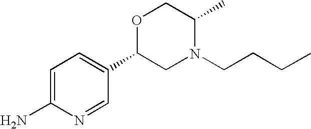

¹H NMR: δ_{H} (400 MHz, CD₃OD) 7.86 (1H, d), 7.49 (1H, dd), 6.56 (1H, d), 4.44 (1H, m), 3.86 (1H, m), 3.39 (1H, m), 2.99 (1H, m), 2.88 (1H, m), 2.52 (1H, brm), 2.41-2.28 (2H, m), 1.60-1.27 (4H, m), 1.07 (3H, d), 0.96 (3H, t)

MS (APCI⁺) 250 (MH⁺)

### EXAMPLE 11 (Diastereomer 2)

### 5-[(2R,5S)-4-butyl-5-methylmorpholin-2-yl]pyridin-2-amine

Retention time 7.20 min

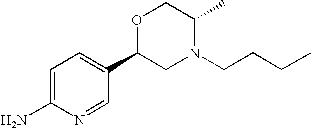

¹H NMR: δ_{H} (400 MHz, CD₃OD) 7.88 (1H, d), 7.48 (1H, dd), 6.55 (1H, d), 4.41 (1H, m), 3.83 (1H, m), 3.72 (1H, d), 2.90 (1H, m), 2.60-2.52 (2H, m), 2.48-2.40 (2H, m), 1.54-1.44 (2H, m), 1.40-1.32 (2H, m), 1.13 (3H, d), 0.94 (3H, t)

MS (APCI⁺) 250 (MH⁺)

### EXAMPLE 12

### 5-{(2R,5S)-5-[(Benzyloxy)methyl]morpholin-2-yl}pyridin-2-amine

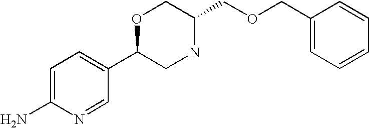

The morpholine from preparation 14 (4.4 g, 9.21 mmol) was dissolved in ethanol (50 mL), hydroxylamine hydrochloride (3.2 g, 46 mmol) was added and the mixture heated at 80° C. overnight (˜16 h). After cooling to room temperature the mixture was diluted with 10% aqueous K₂CO₃ solution (100 mL) and extracted with dichloromethane (2×100 mL). The combined organic fractions were dried over magnesium sulfate, filtered and evaporated to provide a brown oil of crude deprotected 2-amino pyridine intermediate (3.6 g)

This boc-protected morpholine (3.6 g, 9 mmol) was treated with 4M HCl in dioxane (30 mL) and the mixture stirred at room temperature for 4 h. The solvent was then evaporated and the residue treated with 2M sodium hydroxide (100 mL) and extracted with dichloromethane (4×100 mL). The combined organic fractions were dried over magnesium sulphate, filtered and evaporated to a light brown solid which was purified by flash chromatography on silica gel eluting with dichloromethane/methanol/880 NH₃ (93:7:0.5) to provide the title compound as a light brown solid (1.43 g, 51%)

¹H NMR: δ_{H} (400 MHz, CD₃OD) 7.85 (1H, d), 7.45 (1H, dd), 7.36-7.25 (5H, m), 6.54 (1H, d), 4.52 (2H, s), 4.28 (1H, m), 3.97 (1H, m), 3.49-3.38 (3H, m), 3.06 (1H, m), 2.96 (1H, m), 2.76 (1H, m)

MS (ES⁺) 300 (MH⁺)

### EXAMPLE 13

### 5-{(2R,5S)-5-[(Benzyloxy)methyl]-4-propylmorpholin-2-yl}pyridin-2-amine

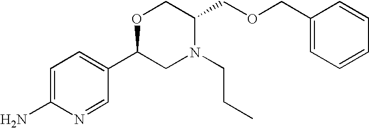

The morpholine from Example 11 (1.4 g, 4.8 mmol) was dissolved in THF (200 mL) and propanal (350 μL, 4.8 mmol) in THF (150 mL) was added dropwise to the stirred mixture. After the addition was complete NaBH(OAc)₃ (1.02 g, 4.8 mmol) was added in one portion and the reaction mixture allowed to stir at room temperature overnight (˜16 h). TLC analysis showed starting material still remaining, so additional NaBH(OAc)₃ (1 g) was added and the reaction mixture stirred for a further 24 h. Saturated aqueous NH₄Cl (200 mL) was added and the organic layer was separated, dried over magnesium sulfate, filtered and evaporated. The residue was purified by flash chromatography on silica gel eluting with dichloromethane/methanol/880 NH₃ (95:5:0.5) to provide the title compound as a light brown oil (540 mg, 33%)

¹H NMR: δ_{H} (400 MHz, CD₃OD) 7.86 (1H, d), 7.46 (1H, dd), 7.36-7.26 (5H, m), 6.53 (1H, d), 4.52 (2H, m), 4.38 (1H, m), 4.00 (1H, m), 3.60-3.53 (2H, m), 3.47-3.42 (1H, m), 2.89 (1H, m), 2.78-2.69 (1H, m), 2.59 (1H, m), 2.32-2.21 (2H, m), 1.60-1.37 (2H, m), 0.84 (3H, t)

MS (ES⁺) 342 (MH⁺)

### EXAMPLES 14-17

### [6-(6-Aminopyridin-3-yl)-4-propylmorpholin-3-yl]methanol

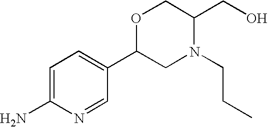

Diol from preparation 16 (1.4 g, 3.9 mmol, 1 eq) was dissolved in dichloromethane (15 mL) and treated with concentrated sulfuric acid (10 mL) at room temperature. The mixture was stirred at room temperature for 2 h before the being quenched by the addition of ice, then basified with 880 NH₃ to pH ˜9. The mixture was extracted with dichloromethane (3×150 mL) and the combined organic layers were dried over magnesium sulfate, filtered and evaporated. The residue was purified by flash chromatography on silica gel eluting with dichloromethane/methanol/880 NH₃ (95:5:0.5 increasing polarity to 93:7:0.5) to afford 110 mg of a light brown oil of the title compound as a mixture of four diastereoisomers.

The diastereoisomers were separated by HPLC on a Chiralpak AD column, mobile phase 20:80 IPA/Hexane with 0.1% DEA affording four stereoisomers

### EXAMPLE 14

Stereoisomer 1 (Retention Time: 9.50 min) Trans Enantiomer 1

¹H NMR: δ_{H} (400 MHz, CD₃OD) 7.86 (1H, d), 7.49 (1H, dd), 6.55 (1H, d), 4.40 (1H, m), 4.05 (1H, m), 3.71 (1H, m), 3.55 (2H, m), 2.93 (1H, m), 2.82 (1H, m), 2.47 (1H, m), 2.34 (1H, m), 2.26 (2H, m), 2.27-1.42 (2H, m), 0.90 (3H, t)

### EXAMPLE 15

Stereoisomer 2 (Retention Time: 11.90 min) cis Enantiomer 1

¹H NMR (400 MHz, CD₃OD) δ(ppm): 0.9 (t, 3H), 1.5-1.7 (m, 2H), 2.5-2.8 (m, 5H), 3.7-4.0 (m, 3H), 4.05-4.15 (m, 1H), 4.4-4.55 (m, 1H), 6.6 (d, 1H), 7.5 (d, 1H), 7.85 (s, 1H)

### EXAMPLE 16

Stereoisomer 3 (Retention Time: 16.60 min) cis Enantiomer 2

¹H NMR (400 MHz, CD₃OD) δ(ppm): 0.9 (t, 3H), 1.5-1.7 (m, 2H), 2.5-2.8 (m, 5H), 3.7-4.0 (m, 3H), 4.05-4.15 (m, 1H), 4.4-4.55 (m, 1H), 6.6 (d, 1H), 7.5 (d, 1H), 7.85 (s, 1H)

### EXAMPLE 17

Stereoisomer 4 (Retention Time: 19.70 min) trans Enantiomer 2

¹H NMR: δH (400 MHz, CD₃OD) 7.86 (1H, d), 7.49 (1H, dd), 6.55 (1H, d), 4.40 (1H, m), 4.05 (1H, m), 3.71 (1H, m), 3.55 (2H, m), 2.93 (1H, m), 2.82 (1H, m), 2.47 (1H, m), 2.34 (1H, m), 2.26 (2H, m), 2.27-1.42 (2H, m), 0.90 (3H, t)

### EXAMPLES 18-19

### 4-methyl-5-(4-Propylmorpholin-2-yl)pyridin-2-amine

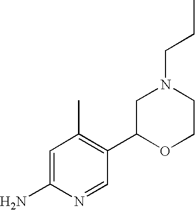

The diol from preparation 21 (950 mg, 3.7 mmol) was dissolved in dichloromethane (15 mL) and treated with concentrated sulfuric acid (7 mL) at room temperature and the mixture stirred for a further 2 h. The reaction was then quenched the addition of ice, then basified by the dropwise addition of 880 NH₃ until the pH ˜9. The mixture was then extracted with dichloromethane (4×50 mL) and the combined organics dried with magnesium sulfate, filtered and evaporated to provide the title compound as a pale brown oil (700 mg, 79%)

¹H NMR: δH (400 MHz, CD₃OD) 7.85 (1H, d), 6.38 (1H, d), 4.60 (1H, m), 3.99 (1H, m), 3.78 (1H, m), 2.92-2.82 (2H, m), 2.38 (2H, m), 2.28-2.18 (4H, m), 2.12 (1H, m), 1.62-1.50 (2H, m) 0.93 (3H, t)

MS (APCI⁺) 236 (MH⁺)

The racemic morpholine was subjected to HPLC using a Chiralcel OD-H column eluting with acetonitrile. This afforded the two enantiomers.

### EXAMPLE 18 (Enantiomer 1)

Retention time: 5.1 min

¹H NMR: δ_{H} (400 MHz, CD₃OD) 7.85 (1H, d), 6.38 (1H, d), 4.60 (1H, m), 3.99 (1H, m), 3.78 (1H, m), 2.92-2.82 (2H, m), 2.38 (2H, m), 2.28-2.18 (4H, m), 2.12 (1H, m), 1.62-1.50 (2H, m) 0.93 (3H, t)

MS (APCI⁺) 236 (MH⁺)

### EXAMPLE 19 (Enantiomer 2)

Retention time: 6.5 min

¹H NMR: δ_{H} (400 MHz, CD₃OD) 7.85 (1H, d), 6.38 (1H, d), 4.60 (1H, m), 3.99 (1H, m), 3.78 (1H, m), 2.92-2.82 (2H, m), 2.38 (2H, m), 2.28-2.18 (4H, m), 2.12 (1H, m), 1.62-1.50 (2H, m) 0.93 (3H, t)

MS (APCI⁺) 236 (MH⁺)

### EXAMPLE 20

### 3-Methyl-5-[(5S)-5-methyl-4-propylmorpholin-2-yl]pyridin-2-amine

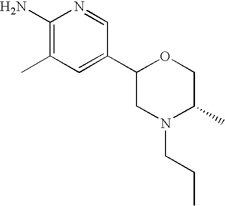

The diol from preparation 29 (200 mg, 0.74 mmol) was dissolved in dichloromethane (4 mL) and treated with concentrated sulfuric acid (2 mL) at room temperature and the mixture stirred for a further 2 h. The reaction was then quenched the cautious addition of water, then basified by the dropwise addition of 880 NH₃ until the pH ˜9. The mixture was then extracted with dichloromethane (3×70 mL) and the combined organics dried with magnesium sulfate, filtered and evaporated. The residue was purified by flash chromatography on silica gel to provide the title compound as a clear oil as a mixture of diastereoisomers (35 mg, 19%)

¹H NMR: δ_{H} (400 MHz, CD₃OD) 7.77 (1H, d), 7.38 (1H, d), 4.41 (1H, m), 3.88-3.70, 2.95-2.72 (3H, 2×m), 2.57 (1H, m), 2.50-2.35 (2H, m), 2.29-2.19 (1H, m), 2.11 (3H, 2×s), 1.61-1.39 (2H, m), 1.18-1.00 (3H, 2×d), 0.91 (3H, m)

MS (ESI⁺) 250 (MH⁺)

### EXAMPLES 21 AND 22

The diol from preparation 24 (990 mg, 3.9 mmol) was dissolved in dichloromethane (10 mL) and treated with concentrated sulfuric acid at room temperature. The mixture was left stirring for 2 h before being quenched by the addition of ice and the basified by the addition of 880 NH₃ to pH ˜9. The mixture was then extracted with dichloromethane (3×150 mL), the combined organics dried over magnesium sulfate, filtered and the solvents evaporated. The residue was purified by flash chromatography on silica gel eluting with dichloromethane/methanol/880 NH₃ (95:5:0.5) to provide the title compound as a mixture of diastereoisomers (470 mg, 51%).

MS (ES⁺) 236 (MH⁺)

The diastereoisomers were split using chiral HPLC on a Chiralcel OD-H column eluting with 30% IPA in hexanes with 0.1% diethylamine. To afford:

### EXAMPLE 21 (Diastereomer 1)

### 5-[(2S,5S)-4,5-Diethylmorpholin-2-yl]pyridin-2-amine

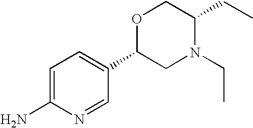

retention time: 4.1 min

¹H NMR: δ_{H} (400 MHz, CD₃OD) 7.88 (1H, d), 7.48 (1H, dd), 6.57 (1H, d), 4.43 (1H, m), 3.98 (1H, d), 3.77 (1H, m), 2.67-2.54 (1H, m), 1.60 (1H, m), 1.11 (3H, t), 0.96 (3H, t)

MS (ES⁺) 236 (MH⁺)

### EXAMPLE 22 (Diastereomer 2)

### 5-[(2R,5S)-4,5-Diethylmorpholin-2-yl]pyridin-2-amine

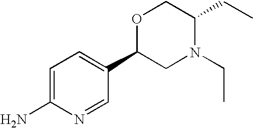

Retention time: 7.3 min

¹H NMR: δ_{H} (400 MHz, CD₃OD) 7.87 (1H, d), 7.47 (1H, dd), 6.56 (1H, d), 4.40 (1H, m), 3.98 (1H, m), 3.43 (1H, m), 3.01-2.90 (2H, m), 2.44 (1H, m), 2.33 (1H, m), 2.25 (1H, m), 1.78 (1H, m), 1.32 (1H, m), 1.06 (3H, t), 0.93 (3H, t)

MS (ES⁺) 236 (MH⁺) □

### EXAMPLE 23

### 5-(1-Propylazetidin-3-yl)pyridin-2-amine

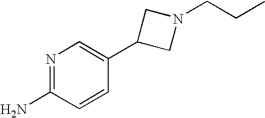

The aminopyridine imine from preparation 35 (95 mg, 0.267 mmol, 1.0 eq) was dissolved in EtOH (2 ml), 10% Pd/C (10 mg) and ammonium formate (168 mg, 2.67 mmol, 10 eq) was added and the mixture heated to a gentle reflux for 3 h. Further 10% Pd/C (10 mg) and ammonium formate (168 mg, 2.67 mmol, 10 eq) added and the mixture heated to reflux for 48 h. The catalyst was filtered off through arbocel, and washed with EtOH. The filtrate was evaporated in vacuo to give a colourless oil. This material was dissolved in THF (5 ml), 2M HCl (aq) added and stirred at r.t. for 3 h. The mixture was evaporated in vacuo and basified with K₂CO₃ (10% w/v aq) and extracted with CH₂Cl₂ (3×20 ml), dried (MgSO₄), filtered and evaporated to give a colourless oil. This oil was purified by flash chromatography on silica gel with a gradient elution from 100% CH₂Cl₂ to 90:10:1 CH₂Cl₂:MeOH:NH₄OH to give the product as a colourless oil which solidified on standing (21 mg, 41%)

Tlc Rf=0.24 (90:10:1 CH₂Cl₂:MeOH:NH₄OH UV visualization)

MS (APCI+) 192 (MH+)

¹H NMR: δ_{H} (400 MHz, CD₃OD) 7.95 (1H, s), 7.45 (1H, d), 6.5 (1H, d), 4.2-4.6 (2H, br s), 3.5-3.8 (3H, m), 3.05 (2H, t), 2.45 (2H, t), 1.3-1.5 (2H, m), 0.9 (3H, t)

### EXAMPLES 24 AND 25

### 5-(2R,5S)-4-Ethyl-5-methylmorpholin-2-yl)-pyridin-2-ylamine & 5-(2S,5S)-4-Ethyl-5-methylmorpholin-2-yl)-pyridin-2-ylamine

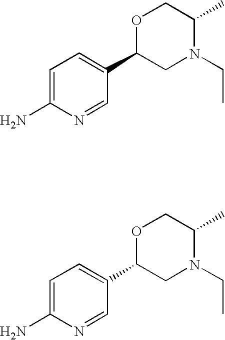

The morpholine from preparation 38 (1 g, 4.17 mmol) was dissolved in CH₂Cl₂ (15 ml) and concentrated sulfuric acid (7.5 ml) was added. The mixture was stirred at r.t. for 2 h, basified by cautious addition of 0.880 NH₃, and extracted with CH₂Cl₂ (2×200 ml), the organics combined, dried over magnesium sulphate, filtered and purified by flash chromatography on silica gel eluting with CH₂Cl₂:MeOH:NH₄OH 97:3:1 to give the title compound as a light brown oil (560 mg 61%).

The diastereoisomers were separated on a Chiralcel OD-H column (500*50 mm) with a mobile phase of 20% IPA, 80% hexane, 0.1% DEA at a flow rate of 80 ml/min to give: Diasteroisomer 1—retention time 5.47 min (Example 24, (2S,5S) diastereoisomer)

¹H NMR: δ_{H} (400 MHz, CD₃OD) 7.88 (1H, s), 7.46-7.52 (1H, m), 6.58 (1H, d), 4.40-4.46 (1H, m), 3.84-3.92 (1H, m), 3.75-3.79 (1H, m), 2.91-2.98 (1H, m), 2.47-2.60 (4H, m), 1.08-1.18 (m, 6H)

MS (APCI+) 222 (MH+)

Diasteroisomer 2—retention time 7.96 min (Example 25, (2R,5S) diastereoisomer)

¹H NMR: □_{H} (400 MHz, CD₃OD) 7.88 (1H, s), 7.44-7.50 (1H, m), 6.56 (1H, d), 4.40-4.46 (m, 1H), 3.80-3.88 (1H, m), 3.28-3.41 (1H, m), 2.88-3.00 (2H, m), 2.35-2.52 (2H, m), 2.16-2.24 (1H, m), 1.00-1.08 (m, 6H)

MS (APCI+) 222 (MH+)

### EXAMPLES 26 & 27

### (+)-5-(4-propylmorpholin-2-yl)-1,3-thiazol-2-amine & (−)-5-(4-propylmorpholin-2-yl)-1,3-thiazol-2-amine

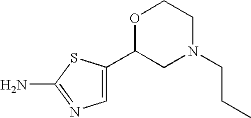

To 2-(2-bromo-1,3-thiazol-5-yl)-4-propylmorpholine (2.5 g 8.56 mmol) in ethylene glycol (60 ml) at −78° C. was added Cu₂O (61 mg, 0.43 mmol, 0.05 eq) and NH₃(1) (20 ml) in a bomb. The vessel was sealed, and heated to 80° C. for 18 h. The vessel was allowed to cool, vented, and partitioned between EtOAc (2×200 ml) and water (100 ml), organic layers combined, dried over MgSO₄ and solvent evaporated to give a brown oil This material was chromatographed on an Isco Companion Combiflash autochromatography system with a gradient elution from 99/1/0.1 CH₂Cl₂/MeOH/NH₃ to 95/5/0.5 CH₂Cl₂/MeOH/NH₃ to give the product as a brown oil (1.1 g).

This material was separated by HPLC on a chiralcel OJ column (250*21.5) with a mobile phase of 70:30 Hexane:IPA at a flow rate of 18 ml/min to give two enantiomers.

Enantiomer 1 retention time 5.140 min

¹H NMR (400 MHz, CD₃OD) δ(ppm): 6.99 (s, 1H), 4.63 (d, 1H), 3.87-3.93 (m, 1H), 3.70-3.77 (m, 1H), 2.95 (d, 1H), 2.78 (d, 1H), 2.31-2.39 (m, 2H), 2.10-2.23 (m, 2H), 1.48-1.60 (m, 2H), 0.92 (t, 3H)

M/S APCI+228 (MH+)

Optical rotation [α]^{D}₂₅ +48.45 (c=1.45 mg/ml MeOH)

Elemental analysis +0.55H₂O Total MW=237.24

Calculated C(50.63), H(7.69), N(17.71)

Actual C(50.90, 50.79), H(7.48, 7.51), N(17.35, 17.38)

Enantiomer 2 retention time 10.750 min

¹H NMR (400 MHz, CD₃OD) δ(ppm): 6.99 (s, 1H), 4.63 (d, 1H), 3.87-3.93 (m, 1H), 3.70-3.77 (m, 1H), 2.95 (d, 1H), 2.78 (d, 1H), 2.31-2.39 (m, 2H), 2.10-2.23 (m, 2H), 1.48-1.60 (m, 2H), 0.92 (t, 3H)

M/S APCI+228 (MH+)

Optical rotation [α]^{D}₂₅ −43.56 (c=2.6 mg/ml MeOH)

Elemental analysis +1H₂O Total MW=245.35

Calculated C(48.96), H(7.81), N(17.13)

Actual C(49.05, 49.07), H(7.83, 7.85), N(17.00, 16.99)

The following preparations illustrate the synthesis of certain intermediates used in the preparation of the preceding examples:

### Preparation 1

### 5-Bromo-2-(2,5-dimethyl-pyrrol-1-yl)-pyridine

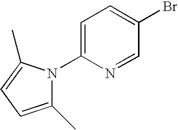

2,5-hexanedione (46.2 g, 0.41 mol) was added to a suspension of 2-amino-5-bromopyridine (50.0 g, 0.29 mol) and the reaction heated to reflux for 24 hours under Dean and Stark conditions. para-Toluenesulfonic acid (100 mg) was added and the reaction was refluxed for a further 18 hours. 8 mL of water was removed, so the reaction was cooled to room temperature, washed with water (100 mL) and passed through a plug of silica gel, eluting with toluene. The eluent was concentrated in vacuo and the residue dissolved in pentane:dichloromethane (1:1 by volume) and passed through a plug of silica gel, eluting with pentane:dichloromethane (1:1 by volume). The eluent was concentrated in vacuo to give a red liquid, which solidified on standing. The solid was recrystallised (isopropanol) to give the title compound as a pale yellow solid (54.4 g).

¹H NMR (400 MHz, CDCl₃): δ_{H} 8.66 (1H, s), 7.93-7.92 (1H, d), 7.13-7.11 (1H, d), 5.91 (2H, s), 2.13 (6H, s).

LRMS (thermospray): m/z [M+H]⁺252.

### Preparation 2

### 2-Chloro-1-[6-(2,5-dimethyl-pyrrol-1-yl)-pyridin-3-yl]-ethanone

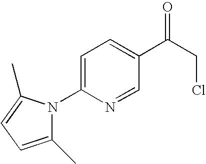

A solution of 2.5 M n-butyl lithium in hexanes (35 mL, 87.6 mmol) was added to a solution of the bromide from preparation 1 (20.0 g, 79.7 mmol) in tert-butylmethylether (300 mL) at −78° C. under nitrogen over 10 minutes. The reaction was stirred for a further 10 minutes and 2-chloro-N-methoxy-N-methylacetamide (12.1 g, 87.6 mmol) in tert-butylmethylether (40 mL) was added slowly. The reaction was stirred at −78° C. for 20 minutes and then 1M hydrochloric acid (200 mL) was added. The mixture was allowed to warm to room temperature, stirred for 2 hours and the organic phase separated. The aqueous phase was extracted with tert-butyl methylether and the combined organic extracts were washed with water (100 mL), saturated aqueous sodium chloride (100 mL) and 1 M sodium hydroxide (100 mL). The organic phase was dried (sodium sulfate), concentrated in vacuo and the residual oil purified by flash column chromatography on silica gel eluting with pentane:dichloromethane:methanol (75:25:0 changing to 0:99:1, by volume). The residue was recrystallised from pentane:dichloromethane to give the title compound as a yellow solid. (14.37 g, 73%)

¹H NMR (400 MHz, CDCl₃): δ_{H} 9.11 (1H, s), 8.34-8.33 (1H, d), 7.32-7.30 (1H, d), 5.91 (2H, s), 4.66 (2H, s), 2.17 (6H, s)

LRMS (electrospray): m/z [M−H]⁺247.

### Preparation 3

### 2-(2,5-Dimethyl-pyrrol-1-yl)-5-[(2R)-oxiranyl]pyridine

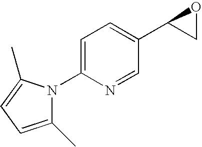

A solution of the chloride from preparation 2 (12.0 g, 48.1 mmol) in tetrahydrofuran (20 ml) was slowly added to a solution of (−)-B-chlorodiisopinocampheylborane (20.1 g, 62.5 mmol) in tert-butylmethylether (15 mL) and tetrahydrofuran (30 mL) at −30° C. under nitrogen. The reaction was stirred for 6 hours at −30° C. and then sodium perborate tetrahydrate (7.4 g, 48.1 mmol) followed by tert-butylmethylether (50 mL) were added. The reaction was stirred at room temperature for 18 hours, treated with 2M aqueous sodium hydroxide (190 mL) and stirred for a further 6 hours. The organic phase was separated and the aqueous phase extracted with further tert-butylmethylether (50 mL). The combined organic extracts were washed with 1 M aqueous sodium hydroxide (50 mL), saturated aqueous sodium chloride (50 mL), dried (sodium sulfate) and concentrated in vacuo. The residue was purified by flash column chromatography on silica gel eluting with pentane:dichloromethane (80:2 changing to 100:0, by volume) to give the crude epoxide (65% b/w, 11.0 g), which was used without further purification.

¹H NMR (400 MHz, CDCl₃): δ_{H} 8.58 (1H, brs), 7.68-7.66 (1H, dd), 7.22-7.20 (1H, d), 3.97-3.96 (1H, m), 3.26-3.24 (1H, m), 2.91-2.89 (1H, m), 2.12 (6H, s)

LRMS (electrospray): m/z [M+H]+215, [M+Na]+, 237.

### Preparation 4

### (1R)-2-(Benzylamino)-1-[6-(2,5-dimethyl-1H-pyrrol-1-yl)pyridin-3-l]ethanol

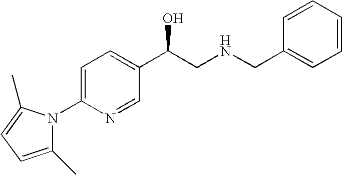

The epoxide from Preparation 3 (2.66 g, 12 mmol) was dissolved in DMSO (30 mL), treated with benzylamine (1.62 mL, 15 mmol) and the mixture heated to 90° C. overnight (˜16 h). After cooling to room temperature the reaction mixture was evaporated under high vacuum at 60° C. to remove most of the DMSO. The residue was diluted with ethyl acetate (150 mL) and washed with water (100 mL). The organic layer was separated and the aqueous layer re-extracted with ethyl acetate (100 mL). The combined organic fractions were dried (MgSO₄), filtered and evaporated to give the title compound as a yellow oil (3.29 g, 84%)

¹H NMR (400 MHz, CDCl₃) 8.55 (1H, s), 7.85 (1H, d), 7.35-7.25 (5H, m), 7.2 (1H, d), 5.9 (2H, s), 4.8 (1H, m), 3.89 (2H, s), 3.01 (1H, dd), 2.78 (1H, t), 2.1 (6H, s)

MS (APCI⁺) 322 (MH⁺)

### Preparation 5

### N-Benzyl-2-chloro-N-{(2R)-2-[6-(2,5-dimethyl-1H-pyrrol-1-yl)pyridin-3-yl]-2-hydroxyethyl}acetamide

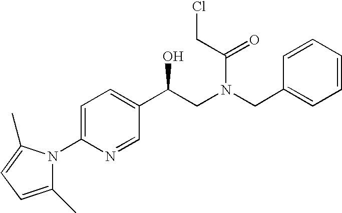

The amino alcohol from Preparation 4 (3.22 g, 10 mmol) was dissolved in dichloromethane (100 mL) sodium hydroxide (2 g, 50 mmol) in an aqueous solution (35 mL) was added and the mixture stirred vigorously. Chloroacetylchloride (0.96 mL, 12 mmol) was added dropwise and stirring was then continued at room temperature overnight (˜16 h). The reaction mixture was then diluted with dichloromethane (200 mL) and water (100 mL). The organic layer was separated, dried (MgSO₄), filtered and evaporated to give a brown oil (4.35 g). The ¹H NMR spectrum indicated that a mixture of chloroamide and morpholinone (product of preparation 5) was formed, so the mixture taken on forward without further purification.

MS (APCI⁺) 398 (MH⁺, chloro amide), 362 (MH⁺, cyclised morpholinone)

### Preparation 6

### (6R)-4-Benzyl-6-[6-(2,5-dimethyl-1H-pyrrol-1-yl)pyridin-3-yl]morpholin-3-one

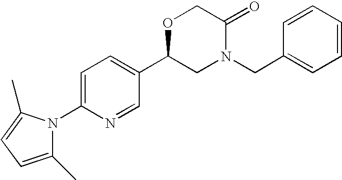

The crude mixture from preparation 5 was dissolved in propan-2-ol (100 mL), water (5 mL) was added followed by potassium hydroxide (673 mg). The mixture was stirred vigorously at room temperature over night. The reaction mixture was then partitioned between ethyl acetate (200 mL) and water (150 mL). The organic layer was separated and washed with brine (150 mL), dried (MgSO₄), filtered and evaporated to afford a dark orange oil. Purification by flash chromatography on silica gel eluting with dichloromethane/methanol 99:1 afforded the title compound as a yellow oil (69%)

¹H NMR (400 MHz, CDCl₃) 8.52 (1H, s), 7.79 (1H, d), 7.30 (5H, m), 7.20 (1 h, d), 5.89 (2H, s), 4.89 (1H, dd), 4.76 (1H, d), 4.63-4.42 (3H, m), 3.49 (1H, t), 3.38 (1H, dd), 2.09 (6H, s)

MS (APCI⁺) 362 (MH⁺)

### Preparation 7

### (2R)-4-Benzyl-2-[6-(2,5-dimethyl-1H-pyrrol-1-yl)pyridin-3-yl]morpholine

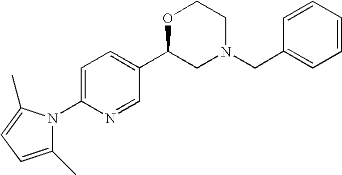

The morpholinone from preparation 6 (2.47 g, 6.8 mmol) was dissolved in tetrahydrofuran (100 mL) and cooled (flask in ice/water bath). Lithium aluminiumhydride (1 M in tetrahydrofuran, 10.2 mL, 10.2 mmol) was added dropwise and after the addition the reaction mixture was allowed to stir at room temperature overnight (˜16 h). The reaction was quenched by the cautious addition of 1M sodium hydroxide (10 mL) then diluted with water (150 mL) and stirred for 10 minutes. Ethyl acetate (200 mL) was added, the organic layer separated, dried over MgSO₄, and evaporated to provide the title compound as a yellow oil (2.09 g, 89%)

¹H NMR (400 MHz, CDCl₃) 8.59 (1H, s), 7.81 (1H, d), 7.3 (5H, m), 7.2 (1H, d), 5.9 (2H, s), 4.69 (1H, d), 4.05 (1H, d), 3.9 (1H, t), 3.6 (2H, s), 3.0 (1H, d), 2.8 (1H, d), 2.35 (1H, t), 2.19 (1H, t), 2.1 (6H, s)

MS (APCI⁺) 348 (MH⁺)

### Preparation 8

### (2S)-2-({(2R)-2-[6-(2,5-Dimethyl-1H-pyrrol-1-yl)pyridin-3-yl]-2-hydroxyethyl}amino) propan-1-ol

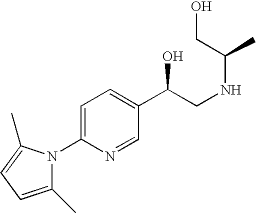

The epoxide from preparation 3 (5.4 g, 20 mmol) was dissolved in DMSO (50 mL) together with (S)-2-aminopropan-1-ol (2.0 g, 20 mmol) and the mixture heated at 90° C. overnight (ca. 16 h). After cooling to room temperature, the mixture was evaporated under high vacuum and the residue purified by flash chromatography on silica gel eluting with dichloromethane/methanol (95:5 increasing polarity to 90:10) to provide the title compound as a pale yellow oil (5.0 g, 75%)

### Preparation 9

### Benzyl (2R,5S)-2-[6-(2,5-dimethyl-1H-pyrrol-1-yl)pyridin-3-yl]-5-methylmorpholine-4-carboxylate

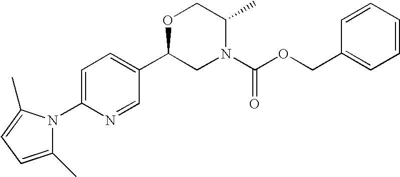

The diol from preparation 8 (5 g, 17.2 mmol) was dissolved in dichloromethane (60 mL) and treated with benzyl chloroformate (2.72 mL, 19 mmol) and triethylamine (2.65 mL, 19 mmol). The mixture was stirred overnight (˜16 h) before being quenched by the addition of 2M sodium hydroxide (100 mL). The mixture was extracted with dichloromethane (2×100 mL) and the combined organic fractions dried (MgSO₄), filtered and evaporated. The residue was purified by flash chromatography on silica gel eluting with a gradient from 25% to 60% ethyl acetate in pentane to afford the CBz-protected intermediate as a light brown oil (2.56 g, 35%)

MS (ES⁺) 446 (MNa⁺)

MS (ES⁻) 422 (M−H⁺)

A sample of the above CBz-proected diol (2 g, 4.7 mmol), was dissolved in toluene (30 mL) together with triphenylphosphine (1.5 g, 5.6 mmol). Diisopropyl azodicarboxylate (1.12 mL, 5.6 mmol) was added dropwise and the reaction mixture was left stirring overnight (−16 h). The reaction mixture was diluted with water (100 mL) and extracted with ethyl acetate (2×100 mL). The combined organic fractions were dried (MgSO₄), filtered and evaporated. The residue was purified by flash chromatography on silica gel eluting with 20% ethyl acetate in pentane to afford the title compound as a clear oil (1.68). 1H NMR shows the sample to contain ˜3 equivalents of diisopropyl hydrazine-1,2-dicarboxylate together with the title compound. Thus ˜40% by weight of material is the title compound, corresponding to an approximate yield of 36%.

¹H NMR: □_{H} (400 MHz, CDCl₃) 8.62 (1H, d), 7.80 (1H, dd), 7.37-7.27 (5H, m), 7.11 (1H, d), 5.88 (2H, s), 5.18 (1H, d), 5.10 (1H, d), 4.26 (1H, m), 4.08 (1H, m), 3.72 (2H, m), 3.46-3.40 (2H, m), 2.09 (6H, s), 1.37 (2H, d)

MS (ES⁺) 406 (MH⁺)

### Preparation 10

### 2-(6-Amino-pyridin-3-yl)-5-methyl-morpholine-4-carboxylic Acid Benzyl Ester

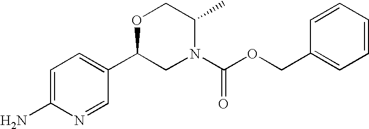

Morpholine from preparation 9 (680 mg, 1. mmol) was dissolved in ethanol (12 mL) and treated with hydroxylamine hydrochloride (600 mg, 8.4 mmol) and the mixture heated at 80° C. overnight (˜16 h). After cooling to room temperature the solvent was evaporated and the residue was purified by flash chromatography on silica gel eluting with dichloromethane in methanol 0% increasing polarity to 2% to provide the title compound as a purple coloured oil (410 mg, 95%).

¹H NMR (400 MHz, CD₃OD) δ_{H} 7.91 (1H, d), 7.43 (1H, dd), 7.37-7.28 (5H, m), 6.52 (1H, d), 5.13 (2H, 2×d), 4.79 (1H, m), 4.12 (1H, m), 4.04 (2H, m), 3.37 (2H, m), 130 (3H, d)

MS (ES⁺) 328 (MH⁺)

### Preparation 11

### (2S)-2-{[(2R)-2-(6-Aminopyridin-3-yl)-2-hydroxyethyl]amino}propan-1-ol

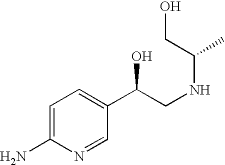

The diol from preparation 8 (1 g, 3.35 mmol) was dissolved in ethanol and treated with hydroxylamine hydrochloride (1.2 g, 16.75 mmol) and the mixture heated at 80° C. overnight (˜16 h). After cooling to room temperature the solvent was evaporated and the residue was purified by flash chromatography on silica gel eluting with dichloromethane/methanol/880 NH₃ (85:15:1 increasing polarity to 82:17:1) to provide the title compound as a light brown oil (670 mg, 95%)

¹H NMR (400 MHz, CD₃OD) δ_{H} 7.91 (1H, s), 7.52 (1H, d), 7.6 (1H, d), 4.72 (1H, d), 3.67 (1H, d), 3.45 (1H, m), 3.1-2.85 (3H, m), 1.15 (3H, d)

MS (ES⁺) 212 (MH⁺) 234 (MNa⁺)

### Preparation 12

### (2R)-3-(Benzyloxy)-2-({(2R)-2-[6-(2,5-dimethyl-1H-pyrrol-1-yl)pyridin-3-yl]-2-hydroxyethyl}amino)propan-1-ol

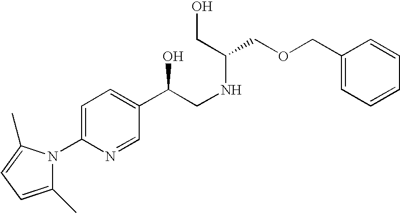

The epoxide from preparation 3 (5.4 g, 25 mmol) was dissolved in DMSO (50 mL) together with (S)-2-amino-3-benzyloxypropan-1-ol (5.0 g, 27.6 mmol) and the mixture heated at 90° C. overnight (ca. 16 h). After cooling to room temperature, the mixture was evaporated under high vacuum to provide a brown oil ˜12 g of desired title compound containing some residual DMSO but of sufficient purity to use in the subsequent stage without further purification.

¹H NMR (400 MHz, CD₃OD) δ_{H} 8.54 (1H, d), 7.99 (1H, dd), 7.25-7.22 (6H, m), 5.81 (2H, s), 4.51 (2H, m), 3.67-3.45 (5H, m), 3.01-2.81 (3H, m), 2.03 (6H, s)

MS (ES⁺) 396 MH⁺

### Preparation 13

### tert-Butyl [(1R)-2-(benzyloxy)-1-(hydroxymethyl)ethyl]{(2R)-2-[6-(2,5-dimethyl-1H-pyrrol-1-yl)pyridin-3-yl]-2-hydroxyethyl}carbamate

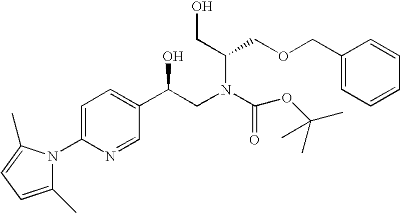

The crude diol from preparation 12 (10 g, ˜25 mmol) was dissolved in dichloromethane (150 mL) and treated with di-tert-butyl dicarbonate (5.52 g, 25 mmol) and the mixture stirred at room temperature overnight (˜16 h). The reaction mixture was diluted with 10% aqueous K₂CO₃ solution (200 mL), the organic layer separated and the aqueous layer extracted with dichloromethane (2×300 mL). The combined organic fractions were dried over magnesium sulfate, filtered and evaporated. The residue was purified by flash chromatography on silica gel eluting with 35% ethyl acetate in pentane increasing polarity of eluent to 50% ethyl acetate in pentane to afford the title compound as a pale yellow oil (6.2 g, 50% yield over 2 steps from preps 12 and 13)

¹H NMR; □_{H} (400 MHz, CD₃OD) 8.55 (1H, d), 8.04-7.95 (1H, m), 7.38-7.23 (6H, m), 5.81 (2H, s), 5.05 (1H, brm) 4.54 (2H, m), 3.93 (1H, brm), 3.83 (1H, brm), 3.78-3.60 (5H, m), 3.44-3.32 (1H, m), 2.05 (6H, s), 1.44 and 1.40 (9H, two singlets)

MS (APCI⁺) 496 (MH⁺)

### Preparation 14

### tert-Butyl (2R,5S)-5-[(benzyloxy)methyl]-2-[6-(2,5-dimethyl-1H-pyrrol-1-yl)pyridin-3-yl]morpholine-4-carboxylate

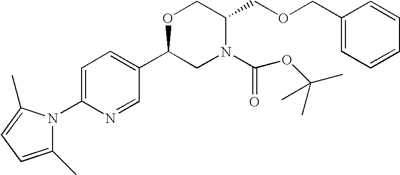

The diol from preparation 13 (6.2 g, 12.5 mmol) was dissolved in toluene (100 mL) and treated with triphenylphosphine (4 g, 15 mmol) at room temperature. Diisopropylazodicarboxylate (DIAD) (3 mL, 15 mmol) was added dropwise and the mixture allowed to stir overnight (−16 h). The reaction mixture was then diluted with water (200 mL), the organic layer separated and the aqueous layer extracted with ethyl acetate (200 mL). The combined organic layers were dried over magnesium sulfate, filtered and evaporated. The residue was purified by flash chromatography on silica gel eluting with 10% ethyl acetate in pentane increasing polarity to 15% ethyl acetate in pentane to provide the title compound as a clear oil (4.4 g, 74%)

¹H NMR: δ_{H} (400 MHz, CDCl₃) 8.64 (1H, d), 7.88 (1H, dd), 7.36-7.27 (5H, m), 7.22 (1H, d), 5.89 (2H, s), 4.96 (1H, m), 4.62 (1H, d), 4.54 (1H, d), 4.28 (1H, m), 4.12 (1H, m) 3.82-3.68 (4H, m), 3.60 (1H, dd), 2.12 (6H, s), 1.44 (9H, s)

MS (APCI⁺) 478 MH⁺

### Preparation 15

### (2R)-3-(Benzyloxy)-2-[{(2R)-2-[6-(2,5-dimethyl-1H-pyrrol-1-yl)pyridin-3-yl]-2-hydroxyethyl}(propyl)amino]propan-1-ol

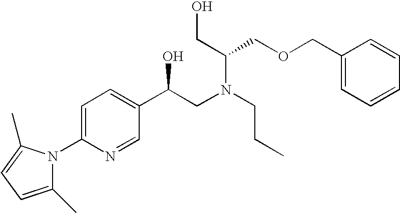

The crude diol from preparation 12 (3 g, 7.6 mmol) was dissolved in dichloromethane and propanal (1.1 mL, 15.2 mmol) and NaBH(OAc)₃ (3.25 g, 15.2 mmol) were added. The reaction mixture was stirred at room temperature overnight (˜16 h) and then solvents were evaporated. The residue was purified by flash chromatography on silica gel eluting with dichloromethane/methanol/880 NH₃ (97:3:0.5) to provide the title compound as a light brown oil still containing ˜3 equivalents of DMSO contamination from the previous stage (4.5 g, corrected for DMSO ˜2.95 g of product, 89% yield)

¹H NMR: δ_{H} (400 MHz, CDCl₃) 8.52 (1H, d), 8.81 (1H, dd), 7.38-7.22 (5H, m), 7.17 (1H, d), 5.86 (2H, s), 4.72 (1H, m), 4.54 (2H, s), 3.48-3.68 (4H, m), 3.16 (1H, m), 2.88-2.95 (1H, m) 2.82-2.55 (3H, m), 2.07 (6H, s), 1.50 (2H, m), 0.87 (3H, t)

MS (APCI⁺) 438 (MH⁺), 460 (MNa⁺)

### Preparation 16

### (2R)-2-[[(2R)-2-(6-˜Aminopyridin-3-yl)-2-hydroxyethyl](propyl)amino]-3-(benzyloxy)propan-1-ol

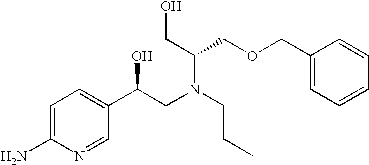

The diol from preparation 15 (2.95 g, 6.7 mmol) was dissolved in ethanol (50 mL) treated with hydroxylamine hydrochloride (2.34 g, 33.7 mmol) and the mixture heated to 80° C. overnight (˜16 h). After cooling to room temperature the solvents were evaporated and the residue purified by flash chromatography on silica gel eluting with dichloromethane/methanol/880 NH₃ (95:5:0.5 increasing polarity to 91:9:0.5) to afford the title compound as a light brown oil (1.4 g, 58%)

¹H NMR: δ_{H} (400 MHz, CD₃OD) 7.82 (1H, d), 7.46 (1H, dd), 7.38-7.22 (5H, m), 6.57 (1H, d), 4.57-4.44 (3H, m), 3.63-3.46 (4H, m), 3.07 (1H, m), 2.77 (2H, d), 2.71-2.53 (2H, m), 1.46 (2H, m), 0.97 (3H, t)

MS (APCI⁺) 360 (MH⁺), 382 (MNa⁺)

### Preparation 17

### 5-Bromo-2-(2,5-dimethyl-1H-pyrrol-1-yl)-4-methylpyridine

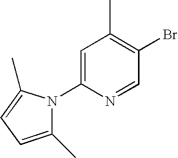

2-Amino-5-bromo-4-methylpyridine [commercially available] (6 g, 32 mmol) was dissolved in toluene (100 mL), hexane-2,5-dione (5.3 mL, 45 mmol) and para-toluene sulfonic acid monohydrate (50 mg) were added and the mixture heated at reflux with a Dean-Stark apparatus fitted. When collection of water ceased the reaction mixture was cooled and diluted with water (50 mL) and 10% aqueous K₂CO₃ solution (50 mL), the organic layer was separated and the aqueous layer extracted with ethyl acetate (200 mL). The combined organic fractions were dried over magnesium sulphate, filtered and evaporated. The residue was purified by flash chromatography on silica gel eluting with 5% ethyl acetate in pentane to afford the title compound as a pale yellow oil (8 g, 95%)

¹H NMR: δ_{H} (400 MHz, CDCl₃) 8.62 (1H, s), 7.11 (1H, s), 5.90 (2H, s), 2.45 (3H, s), 2.15 (6H, s)

MS (ESI⁺) 267 (MH⁺)

### Preparation 18

### 4-Propylmorpholin-2-one

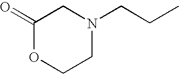

Methyl 2-bromoacetate (50 mL, 0.54 mol, 1 eq) was added slowly to N-propylaminoethanol (62.4 ml, 0.54 mol, 1 eq) and Et₃N (75 ml, 0.54 mol, 1 eq) in toluene at 0° C. and allowed to stir at room temperature overnight. Water (1 L) was added, and the mixture extracted with EtOAc (2×500 mL). Brine (500 mL) was added to the aqueous layer, which was re-extracted with EtOAc (2×500 mL). Organic layers were combined, dried (MgSO₄), filtered and solvent removed in vacuo to give 62.7 g (81%) of title compound as a clear oil.

TLC EtOAc Rf=0.5

M/S (APCI+) 144 (MH+)

¹H NMR (400 Mhz, CD₃OD) δ_{H} 0.9 (t, 3H), 1.4-1.6 (m, 2H), 2.3-2.4 (m, 2H), 2.6-2.7 (m, 2H), 3.3 (s, 2H), 4.4 (m, 2H)

### Preparation 19

### 2-[6-(2,5-Dimethyl-1H-pyrrol-1-yl)-4-methylpyridin-3-yl]-4-propylmorpholin-2-ol

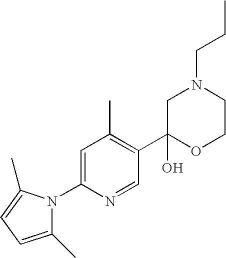

The bromopyridine from preparation 17 (5 g, 18.8 mmol) was dissolved in THF (80 mL) and cooled to −78° C. To the stirred solution was added dropwise tert-butyllithium (22 mL, 37.7 mmol). Immediately after the addition was complete morpholinone (from preparation 18) (2.7 g, 18.8 mmol) was added as a solution in THF (20 mL) and the reaction mixture left stirring at −78° C. for 1 h. The reaction was then quenched the addition on saturated aqueous NH₄Cl solution (100 mL), then extracted with ethyl acetate (100 mL). The organic fraction was dried over magnesium sulphate, filtered and evaporated. The residue was purified by flash chromatography on silica gel eluting with 35% ethyl acetate in pentane increasing polarity of eluent to 40% ethyl acetate in pentane to afford the title compound as a pale yellow oil (1.95 g, 32%)

¹H NMR: δ_{H} (400 MHz, CDCl₃) 8.78 (1H, s), 7.00 (1H, s), 5.86 (2H, s), 5.21 (1H, brs), 4.23 (1H, m), 3.85 (1H, m), 3.03 (1H, m), 2.82 (1H, m), 2.62 (3H, s), 2.56-2.37 (4H, m), 2.08 (6H, s), 1.58 (2H, m), 0.97 (3H, t)

MS (ESI⁺) 330 (MH⁺)

### Preparation 20

### 1-[6-(2,5-Dimethyl-1H-pyrrol-1-yl)-4-methylpyridin-3-yl]-2-[(2-hydroxyethyl)(propyl)amino]ethanol

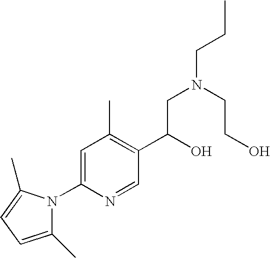

The morpholinol from preparation 19 (1.95 g, 5.9 mmol) was dissolved in ethanol (25 mL) and water (10 mL) and sodium borohydride (900 mg, 23.6 mmol) was added to the stirred mixture at room temperature. Stirring was maintained overnight (−16 h) before the reaction was quenched by the addition of saturated aqueous ammonium chloride (100 mL) and extracted with dichloromethane (2×100 mL). The combined organic fractions were dried over magnesium sulfate, filtered and evaporated. The residue was purified by flash chromatography on silica gel eluting with dichloromethane/methanol/880 NH₃ (96:4:0.5) to afford the title compound as a yellow oil (1.4 g, 71%)

¹H NMR: δ_{H} (400 MHz, CD₃OD) 8.59 (1H, s), 7.16 (1H, s), 5.80 (2H, s), 5.07 (1H, m), 3.67-3.58 (2H, m), 2.82-2.54 (6H, m), 2.47 (3H, s), 2.02 (6H, s), 1.50 (2H, m), 0.90 (3H, t)

MS (ESI⁺) 332 (MH⁺)

### Preparation 21

### 1-(6-Amino-4-methylpyridin-3-yl)-2-[(2-hydroxyethyl)(propyl)amino]ethanol

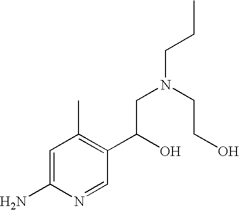

The diol from preparation 20 (1.4 g, 4.22 mmol) was dissolved in ethanol (30 mL) and treated with hydroxylamine hydrochloride (1.12 g, 16.9 mmol), and the mixture heated to reflux over night (˜16 h). After cooling to room temperature the mixture was diluted with 10% aqueous K₂CO₃ solution and extracted with dichloromethane (2×200 mL). The combined organic fractions were dried over magnesium sulfate, filtered and evaporated. The residue was purified by flash chromatography on silica gel eluting with dichloromethane/methanol/880 NH₃ (93:7:1) to afford the title compound as a pale brown oil (950 mg, 89%)

¹H NMR: δ_{H} (400 MHz, CD₃OD) 7.90 (1H, s), 6.39 (1H, s), 4.81 (1H, m), 3.66-3.57 (2H, m), 2.80-2.72 (1H, m), 2.67-2.48 (5H, m), 2.24 (3H, s), 1.58-1.46 (2H, m), 0.91 (3H, t)

MS (ESI⁺) 254 (MH⁺)

### Preparation 22

### (2S)-2-({(2R)-2-[6-(2,5-Dimethyl-1H-pyrrol-1-yl)pyridin-3-yl]-2-hydroxyethyl}amino)butan-1-ol

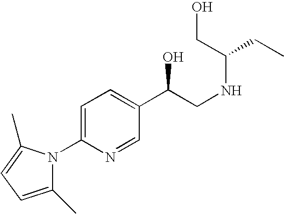

The epoxide from preparation 3 (10.6 g, 49.4 mmol) was dissolved in DMSO (100 mL) together with (S)-2-aminobutan-1-ol (5.6 g, 59.4 mmol) [commercially available] and the mixture heated at 90° C. overnight (ca. 16 h). After cooling to room temperature, the mixture was evaporated under high vacuum to afford a dark oil of the title compound (17.7 g) containing residual DMSO but of sufficient purity to use in the subsequent stage.

¹H NMR: δ_{H} (400 MHz, CDCl₃) 8.56 (1H, d), 7.82 (1H, dd), 7.18 (1H, d), 5.83 (2H, s), 4.77 (1H, m), 3.63 (1H, m), 3.39 (1H, m), 3.04 (1H, m), 2.96-2.78 (2H, brs), 2.70 (1H, m), 2.58 (1H, m), 2.05 (6H, s), 1.54-1.38 (2H, m), 0.92 (3H, t)

MS (ESI⁺) 304 (MH⁺)

### Preparation 23

### (2S)-2-[{(2R)-2-[6-(2,5-Dimethyl-1H-pyrrol-1-yl)pyridin-3-yl]-2-hydroxyethyl}(ethyl)amino]butan-1-ol

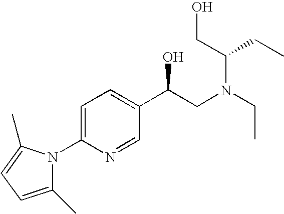

The diol from preparation 22 was dissolved in dichloromethane (50 mL) and treated with ethanal (1.66 mL, 29.6 mmol) and NaBH(OAc)₃ (6.3 g, 29.6 mmol) and the mixture stirred at room temperature over night (˜16 h). The solvents were then evaporated and the residue purified by flash chromatography on silica gel eluting with dichloromethane/methanol/880 NH₃ (97:3:0.5) to afford the title compound as a light brown oil (2.8 g) The material was re-purified by flash chromatography on silica gel eluting with methanol in ethyl acetate 1% increasing polarity to 2% to afford the title compound as a clear oil (1.42 g, 43%)

¹H NMR: δ_{H} (400 MHz, CDCl₃) 8.58 (1H, d), 7.95 (1H, dd), 7.21 (1H, d), 5.87 (2H, s), 4.98 (3H, brm), 3.72 (1H, dd), 3.57 (1H, m), 3.10 (1H, dd), 2.95 (2H, m), 2.78 (2H, m), 2.10 (6H, s), 1.57 (1H, m), 1.43 (1H, m), 1.18 (3H, t), 0.97 (3H, t)

MS (ESI⁺) 332 (MH⁺)

### Preparation 24

### (2S)-2-[[(2R)-2-(6-Aminopyridin-3-yl)-2-hydroxyethyl](ethyl)amino]butan-1-ol

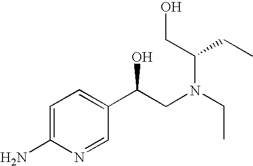

The diol from preparation 23 (1.42 g, 4.3 mmol) was dissolved in ethanol (50 mL) and treated with hydroxylamine hydrochloride (1.5 g, 21.4 mmol) and the mixture heated to 80° C. overnight (˜16 h). After cooling to room temperature, the solvents were evaporated and the residue purified by flash chromatography on silica gel eluting with dichloromethane/methanol/880 NH₃ (91:9:0.5) to afford the title compound as a light brown oil (990 mg, 91%).

¹H NMR: δ_{H} (400 MHz, CD₃OD) 7.88 (1H, d), 7.48 (1H, dd), 6.58 (1H, d), 4.50 (1H, m), 3.42 (2H, m), 2.80 (1H, m), 2.68 (2H, m), 1.83 (2H, m), 1.48 (1H, m), 1.38 (m, 1H), 1.04 (3H, t), 0.92 (3H, t)

MS (ESI⁺) 254 (MH⁺), 276 (MNa⁺)

### Preparation 25

### 5-Bromo-2-(2,5-dimethyl-1H-pyrrol-1-yl)-3-methylpyridine

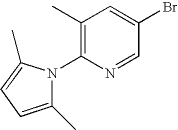

2-Amino-3-methyl-5-bromopyridine (5.86 g, 31.3 mmol) was dissolved in toluene (50 mL), hexane-2,5-dione (5.15 mL, 43.9 mmol) and para-toluene sulfonic acid monohydrate (20 mg) were added and the mixture heated at reflux with a Dean-Stark apparatus fitted. When collection of water ceased, the reaction mixture was evaporated and the residue was purified by flash chromatography on silica gel eluting with 5% ethyl acetate in pentane to afford the title compound as a pale yellow oil (5.1 g, 61%)

¹H NMR: δ_{H} (400 MHz, CDCl₃) 8.51 (1H, d), 7.81 (1H, d), 5.90 (2H, s), 2.01 (3H, s), 1.97 (6H, s)

tlc Rf=0.5 (5% EtOAc:Pentane)

### Preparation 26

### (5S)-5-methyl-4-propylmorpholin-2-one

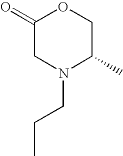

The material from preparation 36 (4 g, 26 mmol) was dissolved in benzene (80 mL), followed by the addition of N-ethyldiisopropylamine (9.07 mL, 52 mmol) and methyl bromoacetate (2.4 mL, 26 mmol). The mixture was heated to reflux with azeotropic removal of water overnight. After cooling to room temperature, the solvent was removed by evaporation, the crude material dissolved in methanol, pre-absorbed onto SiO₂ and purified by flash chromatography on SiO₂ eluting with 40% EtOAc in pentane to afford the title compound as a clear oil (1.78 g).

¹H NMR (CDCl₃, 400 MHz) δ_{H} 0.9 (t, 3H), 1.1 (d, 3H), 1.5 (m, 2H), 2.25 (m, 1H), 2.6 (m, 1H), 2.8 (m, 1H), 3.2 (d, 1H), 3.6 (d, 1H), 4.05 (dd, 1H), 4.3 (dd, 1H)

TLC. Rf=0.18 (50% EtOAc in pentane, UV visualisation)

### Preparation 27

### (5S)-2-[6-(2,5-Dimethyl-1H-pyrrol-1-yl)-5-methylpyridin-3-yl]-5-methyl-4-propylmorpholin-2-ol

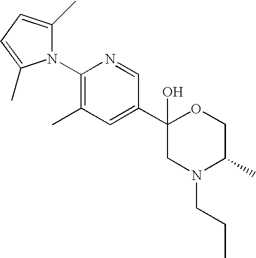

Bromopyridine from preparation 25 (2.5 g, 9.4 mmol) was dissolved in THF (60 mL) and cooled to −78° C. To the stirred solution was added dropwise tert-butyllithium (1.5 M in pentane, 12.6 mL, 18.8 mmol). Immediately after the addition was complete morpholinone (from preparation 26) (1.5 g, 9.4 mmol) was added as a solution in THF (10 mL) and the reaction mixture left stirring at −78° C. for 1 h. The reaction was then quenched by the addition of saturated aqueous NH₄Cl solution (100 mL) then extracted with ethyl acetate (80 mL). The organic fraction was dried over magnesium sulphate, filtered and evaporated. The residue was purified by flash chromatography on silica gel eluting with 10% ethyl acetate in pentane increasing polarity of eluent to 40% then 70% ethyl acetate in pentane to afford the title compound as a pale yellow oil (590 mg, 18%)

¹H NMR: δ_{H} (400 MHz, CDCl₃) 8.70 (1H, s), 7.92 (1H, s), 5.88 (2H, s), 3.77 (2H, brs), 3.0-2.37 (5H, m), 2.02 (3H, s), 1.90 (6H, s), 1.65-1.58 (2H, m), 1.10 (m, 3H), 0.99-0.84 (3H, m)

MS (ESI⁺) 344 (MH⁺)

### Preparation 28

### (2S)-2-[{2-[6-(2,5-Dimethyl-1H-pyrrol-1-yl)-5-methylpyridin-3-yl]-2-hydroxyethyl}(propyl)amino]propan-1-ol

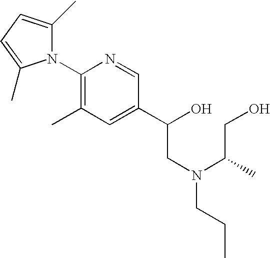

The morpholinol from preparation 27 (600 mg, 1.7 mmol) was dissolved in ethanol (6 mL) and water (3 mL) and sodium borohydride (270 mg, 7 mmol) was added to the stirred mixture at room temperature. Stirring was maintained overnight (˜16 h) before the reaction was quenched by the addition of saturated aqueous ammonium chloride (100 mL) the basified to pH ˜9 with 2M NaOH solution and extracted with dichloromethane (2×200 mL). The combined organic fractions were dried over magnesium sulfate, filtered and evaporated to afford the title compound as a mixture of diastereoisomers as a yellow oil (450 mg, 75%) which was used directly without further purification

MS (ESI+) 346 (MH+), 368 (MNa+)

### Preparation 29

### (2S)-2-[[2-(6-Amino-5-methylpyridin-3-yl)-2-hydroxyethyl](propyl)amino]propan-1-ol

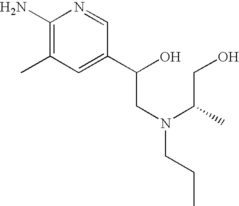

The diol from preparation 28 (420 mg, 1.5 mmol) was dissolved in propanol (5 mL) and water (1.5 mL) treated with hydroxylamine hydrochloride (2.2 g, 31.4 mmol) and triethylamine (2.2 mL, 15.7 mmol), and the mixture heated to reflux for 12 h. After cooling to room temperature the mixture was evaporated, and the residue was purified by flash chromatography on silica gel eluting with dichloromethane/methanol/880 NH₃ (90:10:1) increasing polarity to (85:15:3) to afford a white solid (1.3 g) which was triturated with dichloromethane (3×50 mL), the residual solvent was removed in vacuo to give 700 mg of white solid which was further purified by flash chromatography on silica gel eluting with dichloromethane/methanol/880 NH₃ (92.5:7.5:0.) to afford the title compound as a clear oil (200 mg, 50%)

MS (ESI⁺) 254 (MH⁺)

### Preparation 30

### (2S)-2-[{(2R)-2-[6-(2,5-Dimethyl-1H-pyrrol-1-yl)pyridin-3-yl]-2-hydroxyethyl}(butyl)amino]propan-1-ol

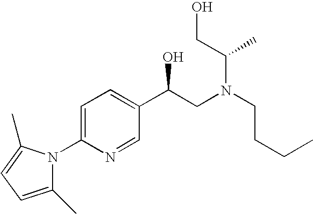

The diol from preparation 8 (1 g, 3.35 mmol, 1 eq) was dissolved in dichloromethane (20 mL) and treated with butanal (910 μl, 10 mmol 3 eq) and NaBH(OAc)₃ (2.1 g, 10 mmol 3 eq) and the mixture stirred at room temperature over night (˜16 h). The reaction was quenched with water (50 ml) and extracted with CH₂Cl₂ (2×100 ml), the organic layers combined, dried (MgSO₄), filtered and evaporated. The residue was purified by flash chromatography on silica gel eluting with dichloromethane/methanol/880 NH₃ (95:5:0.5) to afford the title compound as a clear oil (900 mg 86%)

¹H NMR: δ_{H} (400 MHz, CDCl₃) 8.55 (1H, d), 7.85 (1H, dd), 7.20 (1H, d), 6.88 (2H, s), 4.85 (1H, m), 3.10-2.97 (1H, m), 2.87-2.94 (1H, m), 2.43-2.70 (3H, m), 2.10 (6H, s), 1.22-1.60 (6H, m), 0.87-1.0 (6H, m)

MS (ESI⁺) 346 (MH⁺) 368 (MNa⁺)

Preparation 31

### (2S)-2-[[(2R)-2-(6-Aminopyridin-3-yl)-2-hydroxyethyl](butyl)amino]propan-1-ol

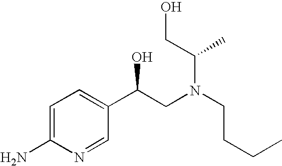

The diol from preparation 30 (900 mg, 2.6 mmol) was dissolved in ethanol (15 mL) and treated with hydroxylamine hydrochloride (905 mg, 13 mmol) and the mixture heated to 80° overnight (˜16 h). After cooling to room temperature, the solvents were evaporated and the residue pre-absorbed onto silica gel and purified by flash chromatography on silica gel eluting with a gradient of dichloromethane/methanol/880 NH₃ (95:5:0.5 to 92:8:0.5) to afford the title compound as a light brown oil (330 mg)

¹H NMR: δ_{H} (400 MHz, CD₃OD) 7.83 (1H, d), 7.50 (1H, dd), 6.59 (1H, d), 4.50 (1H, m), 3.25-3.40 (2H, m), 2.86-2.98 (1H, m), 2.42-2.78 (4H, m), 1.20-1.42 (4H, m), 0.87-1.0 (6H, m)

MS (ESI⁺) 268 (MH⁺), 290 (MNa⁺)

### Preparation 32

### 3-(6-Chloropyridin-3-yl)-azetidine-1-carboxylic acid tert-butyl ester

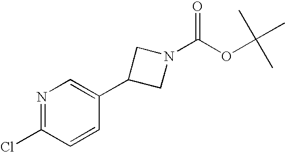

Zinc dust (127 mg, 1.94 mmol, 1.1 eq) was dried for 18 h at 100° C. in vacuo, transferred to a round bottomed flask and heated with a hot air gun under vacuum. The flask was allowed to cool to room temperature and DMF (2 ml) and 1,2-dibromoethane (12 μl, 0.141 mmol, 0.08 eq) added. The mixture was heated to 70° C. for 10 mins, allowed to cool to r.t., and TMSCI (18 μl, 0.141 mmol, 0.08 eq) added dropwise. This mixture was stirred at r.t. for 30 min before dropwise addition of 3-iodoazetidine-1-carboxylic acid tert-butyl ester (Ref SynLett, 1998, 4, 379)(500 mg, 1.766 mmol, 1.0 eq) as a solution in DMF (2 ml). The mixture was stirred at 40° C. for 1 h. 2-chloro-5-iodopyridine was dissolved in DMF (2 ml) and added, followed by Pd₂ dba₃ (32 mg, 0.035 mmol, 0.02 eq) and tri-2-furylphosphine (17 mg, 0.071 mmol, 0.04 eq) and the mixture heated to 70° C. for 4 h.

The mixture was allowed to cool, diluted with Et₂O(40 ml) and NH₄Cl (40 ml, sat'd aq), layers separated, the aqueous layer was re-extracted with Et₂O(20 ml), organics combined, washed with brine (2×30 ml), dried (MgSO4), filtered and evaporated to give a yellow solid.

This solid was flash chromatographed on silica gel with a gradient elution from 100% CH₂Cl₂ to 99:1 CH₂Cl₂:MeOH to give 235 mg of impure product. This material was further purified by fish chromatography on silica get with a gradient elution from 100% toluene to 95:5 toluene:EtOAc to give the title compound as a yellow solid (193 mg, 41%)

Tlc Rf=0.13 (10% EtoAc/Toluene UV visualisation)

MS (APCI+) 269 (MH+)

¹H NMR: δ_{H} (400 MHz, CDCl₃) 8.3 (1H, s), 7.7 (1H, m), 7.35 (1H, d), 4.35 (2H, t), 3.9 (2H, t), 3.65-3.8 (1H, m), 1.45 (9H, s)

### Preparation 33

### 5-Azetidin-3-yl-2-chloropyridine dihydrochloride

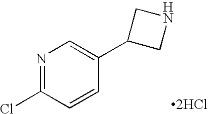

3-(6-Chloropyridin-3-yl)-azetidine-1-carboxylic acid tert-butyl ester (190 mg, 0.707 mmol, 1.0 eq) was dissolved in CH₂Cl₂ (4 ml), cooled to 0° C. and HCl_{(g)} bubbled through for 10 min to give a dark orange solution. This was stirred at r.t. for 72 h. The mixture was evaporated to give a light brown solid, triturated with Et₂O, the resulting solid filtered and dried at 60° C. in vacuo to give the title compound (141 mg, 83%).

Tlc Rf=0.15 (CH₂Cl₂:MeOH:NH₄OH 90:10:1)

MS (APCI+) 169 (MH+)

MS (ESI+) 169 (MH+)

¹H NMR: δ_{H} (400 MHz, CD₃OD) 8.4 (1H, s), 7.95-8.05 (1H, m), 7.55 (1H, d), 4.2-4.5 (5H, m)

### Preparation 34

### 2-Chloro-5-(1-propylazetidin-3-yl)-pyridine

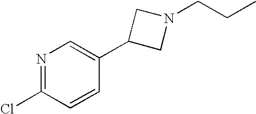

5-Azetidin-3-yl-2-chloropyridine dihydrochloride (131 mg, 0.542 mmol 1.0 eq) was partitioned between CH₂Cl₂ (10 ml) and K₂CO₃ (10 ml, 10% w/v aq), the layers separated, and the aqueous layer re-extracted with CH₂Cl₂ (10 ml). The organic layers were combined, dried (MgSO₄), filtered and evaporated to ca 2 ml volume. Propionaldehyde (79 μl, 1.084 mmol, 2.0 eq) and sodium triacetoxyborohydride (230 mg, 1.084 mmol, 2.0 eq) were added and the mixture stirred at r.t. for 2.5 h. The mixture was quenched with H₂O(0.5 ml) and partitioned between CH₂Cl₂ (10 ml) and K₂CO₃ (10 ml, 10% w/v aq) the layers separated, and the aqueous layer re-extraced with CH₂Cl₂ (10 ml). The organic layers were combined, dried (MgSO₄), filtered and evaporated to give a dark brown oil. This oil was purified by flash chromatography on silica gel with a gradient elution from 100% CH₂Cl₂ to 95:5 CH₂Cl₂:MeOH to give the title compound as a yellow oil (41 mg, 36%)

Tlc Rf=0.39 (CH₂Cl₂:MeOH:NH₄OH 90:10:1 UV visualisation)

MS (APCI+) 211 (MH+)

¹H NMR: δ_{H} (400 MHz, CDCl₃) 8.25 (1H, s), 7.65-7.75 (1H, m), 7.25 (1H, d), 3.6-3.75 (3H, m), 3.1 (t, 2H), 2.4 (t, 2H), 1.3-1.5 (m, 2H), 0.9 (t, 3H)

### Preparation 35

### Benzhydrylidene-[5-(1-ppropylazetidin-3-yl)-pyridin-2-yl]-amine

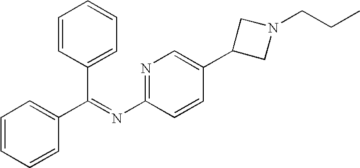

2-Chloro-5-(1-propylazetidin-3-yl)-pyridine (100 mg, 0.475 mmol, 1.0 eq), 1,1-diphenylmethanimine (95 μl, 0.570 mmol, 1.2 eq), palladium (II) acetate (4.4 mg, 0.00475 mmol, 0.01 eq), BINAP (8.7 mg, 0.014 mmol, 0.03 eq) and sodium tert-butoxide (49 mg, 0.665 mmol, 1.4 eq) were combined in toluene (2 ml) and heated to 80° C. for 16 h. Further palladium (II) acetate (4.4 mg, 0.00475 mmol, 0.01 eq) and BINAP (8.7 mg, 0.014 mmol, 0.03 eq) were added and the mixture heated to reflux for 4 h. The mixture was allowed to cool to r.t. and partitioned between EtOAc (25 ml) and K₂CO₃ (20 ml, 10% w/v aq), the layers separated, and the aqueous layer re-extracted with EtOAc (2×25 mlml). The organic layers were combined, dried (MgSO₄), filtered and evaporated to give an orange oil. This oil was purified by flash chromatography on silica gel eluting with a gradient on 100% CH₂Cl₂ to 95:5:0.5 CH₂Cl₂:MeOH:NH₄OH to give the title compound as a yellow oil (110 mg, 66%)

Tlc Rf=0.22 (90:10:1 CH₂Cl₂:MeOH:NH₄OH UV visualization)

MS (APCI+) 356 (MH+)

¹H NMR: δ_{H} (400 MHz, CDCl₃) 8.2 (1H, s), 7.8 (2H, m), 7.0-7.6 (9H, m), 6.55 (1H, d), 3.65-3.8 (3H, m), 3.1 (2H, t), 2.45 (t, 2H), 1.3-1.5 (m, 2H), 0.95 (t, 3H)

CHN +0.75H₂O

Calculated C(78.12) H(7.24) N(11.39)

Observed C(77.85, 78.12) H(7.04, 7.04) N (11.18, 11.28)

### Preparation 36

### (2S)-2-(propylamino)propan-1-ol hydrochloride

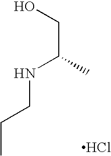

To (2S)-(+)-2-aminopropan-1-ol (19.6 g, 0.26 mol) dissolved in CH₂Cl₂ (500 mL) was added propionaldehyde (20.9 mL, 0.28 mol) followed by pre-dried, powdered 4A molecular sieves (40 g) and the mixture stirred at room temperature overnight. The mixture was filtered through a pad of celite® (filter agent), the pad washed with CH₂Cl₂, and solvent evaporated from the filtrate to give a clear oil. This oil was dissolved in methanol (200 mL) and NaBH₄ was added portionwise over 15 minutes. The mixture was stirred at r.t. overnight, then quenched by cautious addition of 2M aqueous HCl (200 mL), basified by addition of 2M aqueous NaOH (200 mL) and methanol removed by evaporation. Di-tert-butyldicarbonate (115 g, 0.52 mol) was added to the residue followed by 1,4-dioxan (200 mL) and the mixture stirred at r.t. overnight. 1,4-dioxan was removed by evaporation and the residue diluted with water (750 mL) and extracted with CH₂Cl₂ (2×750 mL). The combined organic fractions were dried (MgSO₄), filtered and evaporated giving a clear oil. To this oil was added 4M HCl in 1,4-dioxan (200 mL) and the mixture stirred at r.t. overnight. The solvent was removed by evaporation to give the title compound as a white solid (24 g).

¹H NMR (DMSO, 400 MHz) δ: 0.95 (3H, t), 1.2 (3H, d), 1.6 (2H, m), 2.8 (2H, m), 3.15 (1H, m), 3.5 (1H, brm), 3.6 (1H, m), 5.4 (1H, b), 8.6-8.9 (2H, brd)

M/S (APCI+), 118 (MH+)

### Preparation 37

### (2S)-2-({2-[6-(2,5-Dimethyl-1H-pyrrol-1-yl)pyridin-3-yl]-2-hydroxyethyl}amino) propan-1-ol

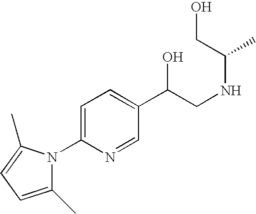

The epoxide from preparation 40 (950 mg, 4.4 mmol) was dissolved in DMSO (10 mL) together with (S)-2-aminopropan-1-ol (380 μL, 4.9 mmol) and the mixture heated at 90° C. overnight (ca. 16 h). After cooling to room temperature, the mixture was evaporated under high vacuum and the residue purified by flash chromatography on silica gel eluting with dichloromethane/methanol/0.880 NH₃ (98:2:0 increasing polarity to 90:10:1) to provide the title compound as a pale yellow oil (780 mg, 67% over 2 steps from preparation 40).

¹H NMR: δ_{H} (400 MHz, CDCl₃) 8.61 (1H, d), 7.86 (1H, dd), 7.21 (1H, d), 5.90 (2H, s), 4.90 (1H, m), 3.68 (1H, m), 3.46 (1H, m), 3.26-2.72 (4H, m), 2.11 (6H, s), 1.14 (3H, 2×d)

MS (APCI⁺) 290 (MH⁺)

### Preparation 38

### (2S)-2-[{2-[6-(2,5-Dimethyl-1H-pyrrol-1-yl)pyridin-3-yl]-2-hydroxyethyl}(ethyl)amino]propan-1-ol (diastereomer mix)

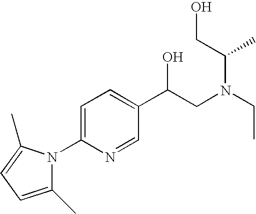

The diol from preparation 37 (1.5 g, 5.2 mmol) was dissolved in dichloromethane (25 mL) and treated with acetaldehyde (870 μL, 15.5 mmol) and sodium triacetoxyborohydride (3.3 g, 15.5 mmol) and the reaction mixture left stirring at room temperature overnight (˜16 h). The reaction mixture was diluted with saturated ammonium chloride solution then basified by the addition of 10% aqueous K₂CO₃ solution, and then extracted with dichloromethane (2×150 mL). The combined organics were dried (MgSO₄), filtered and evaporated. The residue was purified by flash chromatography on silica gel eluting with dichloromethane/methanol. 0.880 NH₃ (93:7:0.5) to afford the title compound as a mixture of diastereoisomers as a slighity impure light brown oil (2.5 g). This material was used directly without further purification.

¹H NMR: δ_{H} (400 MHz, CDCl₃) 8.56 (1H, m), 7.87 (1H, m), 7.21 (1H, d), 5.89 (2H, s), 4.82 (1H, m), 3.77-3.65 (1H, m), 3.18-3.05 (1H, m), 2.95-2.57 (5H, m), 2.10 (6H, s), 1.18-1.10(2×3H, t), 1.00-0.92(2×3H d)

MS (ESI+) 318 (MH+)

### Preparation 39

### (2S)-2-[[2-(6-Aminopyridin-3-yl)-2-hydroxyethyl](ethyl)amino]propan-1-ol (diastereomer mix)

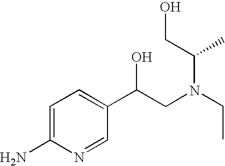

The diol from preparation 38 (2.5 g, 7.8 mmol) was dissolved in ethanol (80 mL) and treated with hydroxylamine hydrochloride (2.7 g, 21.4 mmol) and the mixture heated to 80° C. overnight (˜16 h). After cooling to room temperature, the solvents were evaporated and the residue purified by flash chromatography on silica gel eluting with dichloromethane/methanol/880 NH3 (95:5:0.5 increasing polarity to 90:10:1) to afford the title compound as a light brown oil (1.5 g, 80%)

¹H NMR: δ_{H} (400 MHz, CDCl₃) 7.86 (1H, m), 7.50 (1H, m), 6.58 (1H, m), 4.62-4.49 (1H, m), 3.66-3.29 (2H, m), 3.06-2.41 (7H, m), 1.13-0.86 (6H, m)

MS (ESI+) 240 (MH+)

### Preparation 40

### 2-(2,5-Dimethyl-pyrrol-1-yl)-5-[oxiranyl]pyridine

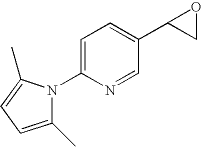

Ethanolamine (0.24 mL, 4 mmol) was added dropwise to a solution of borane.tetrahydrofuran complex (1 M solution in tetrahydrofuran, 8 mL, 8 mmol) in tetrahydrofuran (5 mL) cooled to 0° C. over 15 minutes. The mixture was allowed reach room temperature then the chloride from preparation 2 (1 g, 4 mmol) in tetrahydrofuran was added dropwise to the stirred solution. The reaction mixture was then stirred at room temperature for 30 minutes then quenched by the addition of 2M sodium hydroxide (10 mL) and the reaction mixture stirred for a further 1 hour. The mixture was then extracted with ethyl acetate (2×50 mL), dried (MgSO₄), filtered and evaporated to afford the racemic epoxide as a yellow oil (950 mg). ¹H nmr was as for preparation 3. The material was carried forward directly without further purification.

### Preparation 41

### 4-propyl-2-thiazol-5-yl-morpholin-2-ol

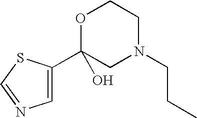

To 2-trimethylsilyl thiazole (9.5 g, 60.5 mmol, 1 eq) in Et₂O (200 ml) at −78° C. was added dropwise n-butyl lithium (36 ml, 2.5M in hexanes, 90.7 mmol, 1.5 eq) and the mixture stirred at −78° C. for 30 min. 4-propylmorpholin-2-one (prepared according to the method described in WO 2004/052372-8.65 g, 60.5 mmol, 1 eq) in Et₂O (50 ml) added over 5 min (temperature increases to −55° C. during addition). The mixture was re-cooled to −78° C. and allowed to stir at −78° C. for 2 h. The reaction was quenched by dropwise addition of water, allowed to warm to r.t. and extracted with EtOAc (2×500 ml) and dichloromethane (2×300 ml). The organic extracts were combined, dried over MgSO₄ and solvent evaporated to give a brown oil. The oil was chromatographed through a SiO₂ column on an Isco Companion Combiflash autochromatography system with a 116 gradient elution from 981210.5 CH₂Cl₂/MeOH/NH₃ to 95/5/0.5 CH₂Cl₂/MeOH/NH₃ to give a pale brown solid (8.5 g, 61%).

¹H NMR (400 MHz, CD₃OD) δ(ppm): 8.91 (s, 1H), 7.91 (s, 1H), 4.13-4.22 (m, 1H), 3.68-3.75 (m, 1H), 2.97 (d, 1H), 2.68-2.80 (m, 1H), 2.26-2.40 (m, 4H), 1.48-1.60 (m, 2H), 0.93 (m, 3H)

M/S APCI+229 (MH+)

Tlc 95/5/0.5 CH₂Cl₂/MeOH/NH₃ Rf=0.35

Tlc EtOAc Rf=0.3

### Preparation 42

### 2-[(2-hydroxyethyl)(propyl)amino]-1-(1,3-thiazol-5-yl)ethanol

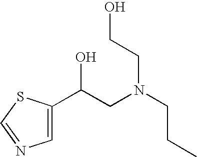

To 4-propyl-2-thiazol-5-yl-morpholin-2-ol (8.5 g, 37.3 mmol) in EtOH (125 ml) and water (125 ml) was added NaBH₄ and the mixture stirred at r.t. for 1 h. The mixture was diluted with water (200 ml) and extracted with dichloromethane (3×250 ml). Organic layers combined, dried over MgSO₄ and solvent evaporated to give a pale oil (6.2 g).

¹H NMR (400 MHz, CD₃OD) δ(ppm): 8.92 (s, 1H), 7.80 (s, 1H), 5.05 (t, 1H), 3.56-3.65 (m, 2H), 2.62-2.80 (m, 4H), 2.51-2.59 (m, 2H), 1.43-1.53 (m, 2H), 0.87 (t, 3H)

M/S APCI+231 (MH+).

t.l.c. 90/10/1 CH₂Cl₂/MeOH/NH₃ Rf 0.45

### Preparation 43

### 4-propyl-2-(1,3-thiazol-5-yl)morpholine

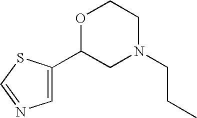

2-[(2-hydroxyethyl)(propyl)amino]-1-(1,3-thiazol-5-yl)ethanol (5.7 g 24.8 mmol) in dichloromethane (20 ml) was treated with concentrated sulphuric acid (50 ml). On complete addition dichloromethane removed in vacuo and resulting mixture heated to 140° C. for 1 h. The mixture was allowed to cool to r.t., poured into ice and cautiously quenched by addition of 0.880 NH₃ with ice cooling maintaining T<20° C. The mixture was extracted with dichloromethane (2×250 ml) dried over MgSO₄ and solvent evaporated to give a brown oil. This material was chromatographed on an Isco Companion Combiflash autochromatography system with an eluant of 981210.5 CH₂Cl₂/MeOH/NH₃ to give the product as a pale brown oil.

¹H NMR (400 MHz, CD₃OD) δ (ppm): 8.94 (s, 1H), 7.84 (s, 1H), 4.93 (dd, 1H), 3.93-4.0 (m, 1H), 3.76-3.84 (m, 1H), 3.07 (d, 1H), 2.81 (d, 1H), 2.35-2.41 (m, 2H), 2.17-2.30 (m, 2H), 1.50-1.62 (m, 2H), 0.94 (t, 3H)

M/S APCI+213 (MH+)

Tlc 95/5/0.5 CH₂Cl₂/MeOH/NH₃ Rf=0.55

### Preparation 44

### 2-(2-bromo-1,3-thiazol-5-yl)-4-propylmorpholine

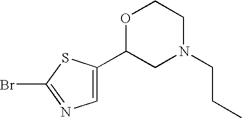

To 4-propyl-2-(1,3-thiazol-5-yl)morpholine (2.9 g 13.7 mmol) in THF (50 ml) at −78° C. was added n-butyl lithium (5.5 ml, 2.5M in hexanes, 13.7 mmol, 1 eq) and allowed to stir at −78° C. for 30 min. A solution of carbon tetrabromide (4.5 g, 13.7 mmol) in THF (20 ml) was added maintaining T<−70° C. during the addition, and the mixture allowed to stir at −70° C. for 1 h. The reaction was quenched by cautious addition of water and allowed to warm to r.t. over 18 h. The mixture was extracted with EtOAc (3×150 ml), dried over MgSO₄ and solvent evaporated to give a brown oil. This material was chromatographed on an Isco Companion Combiflash autochromatography system with a gradient elution from 99/1/0.1 CH₂Cl₂/MeOH/NH₃ to 95/5/0.5 CH₂Cl₂/MeOH/NH₃ to give the product as a brown oil (2.5 g, 63%).

¹H NMR (400 MHz, CD₃OD) δ(ppm): 7.54 (s, 1H), 4.83-4.89 (m, 1H), 3.90-3.96 (m, 1H), 3.70-3.79 (m, 1H), 3.02 (d, 1H), 2.75 (d, 1H), 2.35-2.41 (m, 2H), 2.17-2.30 (m, 2H), 1.50-1.62 (m, 2H), 0.94 (t, 3H)

M/S APCI+293 (MH+)

Tlc 95/5/0.5 CH₂Cl₂/MeOH/NH₃ Rf=0.75

1. Compounds of formula (I): 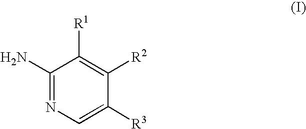
wherein:
R¹ is selected from H and (C₁-C₆)alkyl;
R² is selected from H and (C₁-C₆)alkyl;
R³ is selected from: 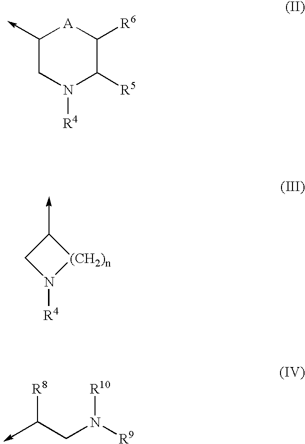
wherein:
A represents O, S or CH₂;
n is 1 or 2;
R⁴ is selected from H and (C₁-C₆)alkyl; wherein said (C₁-C₆)alkyl may be optionally substituted with 1 or 2 substituents each independently selected from (C₁-C₆)alkyl, OR⁷, phenyl, substituted phenyl and heteroaryl;
R⁵ is selected from H and (C₁-C₆)alkyl; wherein said (C₁-C₆)alkyl may be optionally substituted with 1 or 20R⁷ groups;
R⁶ is selected from H and (C₁-C₆)alkyl;
R⁷ is selected from H and (C₁-C₆)alkyl; wherein said (C₁-C₆)alkyl may be optionally substituted with a phenyl, or a substituted phenyl group;
R⁸ is selected from H, methyl, ethyl, methoxy, and ethoxy;
R⁹ represents (C₁-C₆)alkyl; and
R¹⁰ is selected from H and (C₁-C₆)alkyl; wherein said (C₁-C₆)alkyl may be optionally substituted with 1 or 2 substituents each independently selected from OR⁷, phenyl or substituted phenyl;
wherein heteroaryl means a 5 to 7 membered aromatic ring, containing from 1 to 4 heteroatoms, said heteroatom independently selected from O, S and N; said heteroaryl may be optionally substituted with 1 or more substituents each independently selected from (C₁-C₆)alkyl, halo and OR⁷, each substituent may be the same or different; and
wherein substituted phenyl means phenyl substituted with 1 or more substituents each independently selected from (C₁-C₆)alkyl, halo and OR⁷, each substituent may be the same or different;
with the proviso that:
when R¹ and R² are H, R³ is moiety (II), A is O, R⁵ is H or (C₁-C₆)alkyl, and R⁶ is H or (C₁-C₆)alkyl, then R⁴ cannot be n-propyl;
and pharmaceutically acceptable salts, solvate, polymorphs and prodrugs thereof.

2. A compound according to claim 1 wherein R¹ and R² are each independently selected from H and methyl.

3. A compound according to claims 1 or 2 wherein R³ is moiety (II).

4. A compound according to claim 3 wherein A is selected from O or CH₂.

5. A compound according to claim 4 wherein R⁴ is (C₁-C₄) alkyl optionally substituted with phenyl; R⁵ is selected from methyl and ethyl, wherein said methyl and said ethyl may be optionally substituted with an OR⁷ group; and R⁶ is selected from H and methyl.

6. A compound according to claims 1 or 2 wherein R³ is moiety (III).

7. A compound according to claim 6 wherein n is 1 and R⁴ is selected from ethyl, propyl or butyl, said alkyl groups being optionally substituted by a phenyl group.

8. A compound according to claims 1 or 2 wherein R³ is moiety (IV), R⁸ is selected from H and methoxy; and R¹⁰ is selected from H and methyl.

9. A compound according to claim 1 selected from:
5-(Morpholin-2-yl)pyridin-2-amine;
5-[4-(3-Phenylpropyl)morpholin-2-yl]pyridin-2-amine;
5-[(2R, 5S)-5-Methylmorpholin-2-yl]pyridine-2-amine;
5-[(2S,5S)-5-Methyl-4-(3-phenylpropyl)morpholin-2-yl]pyridin-2-amine;
5-[(2S,5S)-4-Butyl-5-methylmorpholin-2-yl]pyridin-2-amine;
5-(2R,5S)-5-[(Benzyloxy)methyl]-4-propylmorpholin-2-yl}pyridin-2-amine;
[6-(6-Aminopyridin-3-yl)-4-propylmorpholin-3-yl]methanol;
4-Methyl-5-(4-Propylmorpholin-2-yl)pyridin-2-amine;
5-[(2S,5S)-4,5-Diethylmorpholin-2-yl]pyridin-2-amine;
5-[(2R,5S)-4,5-Diethylmorpholin-2-yl]pyridin-2-amine; and
5-(2R,5S)-4-Ethyl-5-methylmorpholin-2-yl)-pyridin-2-ylamine.

10. (canceled)

11. A method for treating sexual dysfunction, depression, psychiatric disorders or neurodegeneration comprising the administration compound according to claims 1, 2 or 9.

12. The method according to claim 11 wherein the sexual dysfunction is male erectile dysfunction or female sexual dysfunction.

13. (canceled)

14. (canceled)

15. A pharmaceutical composition comprising a compound according to claims 1, 2 or 9, and a pharmaceutically acceptable diluent or carrier.
